(19) United States
(12) Patent Application Publication
(10) Pub. No.: US 2010/0093638 A1
(43) Pub. Date: Apr. 15, 2010

(54) TREAMENT FOR CHEMICAL SUBSTANCE ADDICTION

(76) Inventors: Suzanne L. Dickson, Askim (SE); Jörgen Engel, Goteborg (SE); Emil Egecioglu, Goteborg (SE); Elisabet Jerlhag, Goteborg (SE)

Correspondence Address: Lynn E. Barber, Post Office Box 16528, Fort Worth, TX, 76162, US

(21) Appl. No.: 12/451,449

(22) PCT Filed: May 12, 2008
(86) PCT No.: PCT/SE2008/050546

§ 371 Date: Nov. 12, 2009

(30) Foreign Application Priority Data
May 14, 2007 (SE) SE0701155

(51) Int. Cl. A61K 38/08; G01N 33/566; A61K 31/4196

(52) U.S. Cl. 514/15; 514/16; 436/501; 514/383

(57) ABSTRACT

The present invention provides a method for the treatment of chemical substance abuse by selectively inhibiting ghrelin activity in humans comprising administering to a human in need thereof a therapeutically-effective amount of a ghrelin receptor ligand (GHS-RL). The ghrelin receptor ligand (GHS-RL) can be selected from the group consisting of a ghrelin receptor antagonist (GHS-RA), a ghrelin receptor inverse agonist (GHS-RIA), and a ghrelin receptor partial agonist (GHS-RPA). More specifically, the invention provides a method for treating alcohol related disorders in humans comprising administering to a human in need thereof a therapeutically-effective amount of a compound which is a ghrelin receptor ligand (GHS-RL), such as a ghrelin receptor antagonist (GHS-RA), a ghrelin receptor inverse agonist (GHS-RIA) and a ghrelin receptor partial agonist (GHS-RPA).

### FIELD OF THE INVENTION

The present invention relates to the treatment of chemical substance addiction, particularly the treatment of alcohol related disorders. More specifically the invention relates to a method for treating chemical substance addiction, especially alcohol-related disorders by administering a compound which blocks ghrelin action.

### BACKGROUND OF THE INVENTION

The World Health Organization (WHO) estimates that there are about 76.3 million with diagnosable alcohol use disorders. From a public health perspective, the global burden related to alcohol consumption, both in terms of morbidity and mortality, is considerable in most parts of the world. Alcohol consumption has health and social consequences via intoxication (drunkenness), alcohol dependence, and other biochemical effects of alcohol. In addition to chronic diseases that may affect drinkers after many years of heavy use, alcohol contributes to traumatic outcomes that kill or disable at a relatively young age, resulting in the loss of many years of life due to death or disability. There is increasing evidence that besides the volume of alcohol, the pattern of the drinking is relevant for the health outcomes. Overall there is a causal relationship between alcohol consumption and more than 60 types of disease and injury. Alcohol is estimated to cause about 20-30% of oesophageal cancer, liver cancer, cirrhosis of the liver, homicide, epileptic seizures, and motor vehicle accidents worldwide (WHO, 2002). Alcohol causes 1.8 million deaths (3.2% of total) and a loss of 58.3 million (4% of total) of Disability-Adjusted Life Years (DALY) (WHO, 2002). Unintentional injuries alone account for about one third of the 1.8 million deaths, while neuro-psychiatric conditions account for close to 40% of the 58.3 million DALYs. The burden is not equally distributed among the countries. Alcohol consumption is the leading risk factor for disease burden in low mortality developing countries and the third largest risk factor in developed countries. In Europe alone, alcohol consumption was responsible for over 55 000 deaths among young people aged 15-29 years in 1999 (Rehm S & Eschmann J, Soz Praventivmed. 2002; 47:48-58).

Estimates of the economic costs of alcohol abuse, collected by the World Health Organization, vary from one to six percent of a country's GDP. One Australian estimate pegged alcohol's social costs at 24 percent of all drug abuse costs; a similar Canadian study concluded alcohol's share was 41 percent. A study quantified the cost to the UK of all forms of alcohol misuse as £18.5-20 billion annually (2001 figures). In Sweden the total costs of alcohol use disorders has been estimated to be as high as 80-100 billion SEK annually.

Current therapeutic strategies for treating alcohol related disorders are entirely unsatisfactory. A more dated approach includes amongst others, disulfiram therapy, that rely on the use of drugs that cause the patient to experience unpleasant effects such as emesis and nausea if they consume alcohol. Such aversive strategies often fail because they do not interfere with the mechanism underlying the addiction process itself. We have previously shown that alcohol activates the cholinergic-mesolimbic dopaminergic reward link, which appear to be a common neurochemical denominator for drugs of abuse and addictive behavior in general. Drugs interfering indirectly with this link have been shown to be of therapeutic value for the treatment of alcohol use disorders, such as acamprosate or naltrexone. However, the therapeutic effect size of these drugs is insufficient, highlighting the urgent need for new therapies for alcohol use disorders. In addition, alcoholism is a heterogeneous disease with a diversified neurochemical basis. This implicates a need for the development of a pharmacological strategy with various modes of interference with the brain reward systems.

Growth hormone-releasing peptides (GHRPs) were first described in 1981 by Bowers and colleagues before the discovery of growth hormone-releasing hormone (GHRH) (Momany F A, et al. Endocrinology 108: 31-39, 1981. Bowers C Y, et al. Endocrinology 1984; 114: 1537-1545). While Bowers' group demonstrated that such peptides could stimulate growth hormone (GH) release from isolated pituitary glands, they almost always reported a greater GH response when the GHRPs were administered in vivo. These data, reported in the early 1980's, suggested that such GHRPs have actions at both the hypothalamus and pituitary. After almost a decade, a non-peptidyl GH secretagogue (GHS) was reported and there have been many additional improvements in potency, bioavailability and pharmacokinetics of GHS (Smith R G, et al. Science 1993; 260: 1640-1643).

After Smith and colleagues identified GHS, they isolated a GHS receptor (GHS-R) cDNA from both the pituitary and hypothalamus (Howard A D, et al. Science 1996; 273: 974-977). In December 1999, the endogenous ligand for GHS-R was identified and named ghrelin (Kojima M, et al. Nature 1999; 402: 656-60). They demonstrated that it is secreted by stomach tissue; and its mRNA is also expressed in the hypothalamus. Thus, the GHS-R now may be thought of as the ghrelin receptor. For a review on this topic see: Bowers C Y, J. Clin. Endocrinol. Metab. 2001: 86: 1464-1469.

Although most GHS and GHRP studies were designed to exploit stimulation of the somatotropic axis, it has been demonstrated that these synthetic molecules induce food intake (Locke W, et al. Life Sci. 1995; 56:1347-1352; Okada K, et al. Endocrinology 1996; 137:5155-5158). Moreover, Bennett et al. (Endocrinology 1997. 8: 4552-4557) demonstrated that GHS-R is highly expressed in the arcuate nucleus. In 1993, we observed an activation of such hypothalamic neurons after peripheral administration of a GHRP (Dickson S L, et al. Neuroscience 1993; 53: 303-306). We also demonstrated that the majority of these activated neurons were those expressing neuropeptide-Y mRNA (Dickson S L and Luckman S M., Endocrinology 1997; 138: 771-777). Ghrelin and GHS have been shown to increase body fat (Tschöp M, et al. Nature 2000; 407: 908-913; Lall S, et al. Biochem Biophys Res Commun. 2001; 280:132-138). A role for ghrelin in the initiation of hunger has been proposed, based on the sharp preprandial rise in ghrelin levels (Cummings D E, et al. Diabetes. 2001; 50:1714-1719). Despite this, circulating ghrelin levels are low in obese patients, suggesting a limited use for ghrelin inhibitors, e.g. ghrelin receptor antagonists (GHS-RAs) and ghrelin receptor inverse agonists (GHS-RIAs) as an anti-obesity therapy. One notable exception is Pradder-Willi patients, where high ghrelin levels are thought to be causal for the compulsive eating behaviour and consequent obesity (Cummings D E, et al. Nature Medicine 2002; 8:643-4).

The behaviours driving animals (and man) to work and seek for food must be highly motivated and to some extent rewarding. Reward for feeding is inter alia regulated by the mesocorticolimbic dopamine system. This neural system, a common denominator of the reward systems, can be activated, causing dopamine release in the nucleus accumbens (N.Acc.), by natural rewards as well as by all dependence-producing drugs. This accumbal dopamine release has been suggested to be responsible for the hedonic feeling of incentives, natural as well as artificial. Additionally, accumbal dopamine release has been shown to be associated with the desire for food during presentation of palatable food stimuli proposing a role of dopamine in the motivation to feed. Furthermore, the dopamine reward systems have been implicated in addictive behaviours such as compulsive overeating, pathological gambling and drug addiction. Additionally, the cholinergic input to the mesoaccumbal dopaminergic neurons in the ventral tegmental area (VTA), i.e. the cholinergic-dopaminergic reward link, has been suggested to mediate reinforcement of natural reward, e.g. food intake, as well as addictive drugs such as alcohol. There is accumulating evidence that the mesolimbic system is a target for ghrelin. In addition to the hypothalamus, the ghrelin receptor GHS-R has also been identified in VTA and LDTg, areas important for the rewarding and reinforcing effects of compulsive addictive behaviours. We have findings indicating that the effects of ghrelin on food intake are partly mediated by the mesolimbic dopamine systems involved in reward-seeking behaviour (Jerlhag E, et al. Addict Biol. 2006 11:45-54; Jerlhag E, et al. Addict Biol. 2007 12:6-16).

Ghrelin receptor ligands (GHS-RL), including ghrelin receptor antagonists (GHS-RA), ghrelin receptor inverse agonists (GHS-RIA) and ghrelin receptor partial agonist (GHS-RPA), are e.g. described in WO 01/87335, WO 01/92292, WO 02/08250, WO 2003/004518, WO 2004/004772, WO 2004/013274, WO 2005/112903, WO 2005/114180, WO 2007/020013, WO 2005/097788, WO 2006/020959, WO 2005/012331, WO 2008/008286, and the corresponding US 2003/0211967, US 2005/0201938, U.S. Pat. No. 6,967,237, US 2003/0186844, US 2002/0187938, US 2006/0276381, US 2006/0257867, U.S. 60/707,941, U.S. 60/787,543, US 2005/0171132, US 2005/0070712, US 2006/0089398, US 2005/0261332, US 2006/0199796, US 2005/0137127, US 2005/0272648, which are hereby incorporated by reference herein.

Ghrelin receptor inverse agonists (GHS-RIA) are e.g. described in Holst B, et al. Mol. Pharmacol. 2006. 70(3): 936-46 and in WO 2007/020013, based on U.S. 60/707,941 and U.S. 60/787,543.

WO 02/08250 discloses peptides with the formula

Gly-Ser-Ser(Octanoyl)-Phe-A

### Where A is —OH, —NH₂, -Leu-Ser-Pro-Glu-B, or -Ala-Lys-Leu-Gln-Pro-Arg-B,

where B is —OH or —NH₂
which are ghrelin receptor antagonists (GHS-RA).

### SUMMARY OF THE INVENTION

The present invention provides a method for the treatment of chemical substance abuse by selectively inhibiting ghrelin activity in humans comprising administering to a human in need thereof a therapeutically-effective amount of a ghrelin receptor ligand (GHS-RL). The ghrelin receptor ligand (GHS-RL) can be selected from the group consisting of a ghrelin receptor antagonist (GHS-RA), a ghrelin receptor inverse agonist (GHS-RIA), and a ghrelin receptor partial agonist (GHS-RPA). More specifically, the invention provides a method for treating alcohol related disorders in humans comprising administering to a human in need thereof a therapeutically-effective amount of a compound which is a ghrelin receptor ligand (GHS-RL) selected from the group consisting of a ghrelin receptor antagonist (GHS-RA), a ghrelin receptor inverse agonist (GHS-RIA) and a ghrelin receptor partial agonist (GHS-RPA).

### DEFINITIONS

For purposes of the present invention, treating or treatment describes the management and care of a patient for the purpose of combating the disease, condition, or disorder. Treating includes the administration of a compound of the present invention to prevent the onset of the symptoms or complications, alleviating the symptoms or complications, or eliminating the disease, condition, or disorder. Treating alcohol-related disorders therefore includes the reduction of alcohol intake, the inhibition of alcohol dependence, interference with the development of the dependence process and relapse prevention

The ghrelin receptor is synonymous to the growth hormone secretagogue receptor (GHS-R).

The cDNA encoding human growth hormone secretagogue receptor has been cloned and designated GHS-R1A. Genbank accession no. U60179. The protein sequence can be found in SwissProt entry Q92847, GHSR_HUMAN.

A growth hormone secretagogue receptor ligand (GHS-RL) is synonymous to a ghrelin receptor ligand. A growth hormone secretagogue receptor antagonist (GHS-RA) is synonymous to a ghrelin receptor antagonist. A growth hormone secretagogue receptor inverse agonist (GHS-RIA) is synonymous to a ghrelin receptor inverse agonist, and a growth hormone secretagogue receptor partial agonist (GHS-RPA) is synonymous to a ghrelin receptor partial agonist.

A ghrelin receptor ligand (GHS-RL), is a compound that binds to the ghrelin receptor (GHS-R), and inhibits and/or stimulates the activity of the receptor and/or competes with the natural ligand for the receptor in a binding assay.

A ghrelin receptor antagonist (GHS-RA), is a compound that partially or fully antagonizes, blocks, or otherwise inhibits the biological action of ghrelin on the ghrelin receptor (GHS-R).

A ghrelin receptor inverse agonist (GHS-RIA) is a compound that decreases the basal constitutive activity of the ghrelin receptor (GHS-R). This term also includes ghrelin receptor partial inverse agonist, which only decreases the basal activity of the receptor to a certain level and not fully.

A ghrelin receptor partial agonist (GHS-RPA), is a compound that increases the functional activity of the ghrelin receptor (GHS-R) to a certain level but not fully, as compared with the full level of activity that can be obtained in the presence full agonist, such as in the presence of ghrelin.

An individual ghrelin receptor ligand (GHS-RL) can acts both an agonist in the absence of ghrelin, and as an antagonist in the presence of ghrelin.

For purposes of this invention, the term “alcohol related disorders” includes, but is not limited to, over-consumption of alcohol, binge drinking, development of alcohol dependence, withdrawal of alcohol, craving for alcohol and relapse.

The term ‘administering’ or ‘administration’ as used herein includes any means for introducing a GHS-RL, a GHS-RA, a GHS-RPA or a GHS-RIA into the body such that the substance is able to interact with the GHS-R or secreted ghrelin. Preferred routes of administration will introduce the substance into the systemic circulation. Examples include but are not limited to oral, nasal. transdermal, or subcutaneous, intravenous, and intramuscular injection.

The active agents of the present invention are administered to a human, in accord with known methods, such as intravenous administration as a bolus or by continuous infusion over a period of time, by intramuscular, intraperitoneal, subcutaneous, intra-articular, intrasynovial, intrathecal, intraocular, intralesional, intranasal, oral, topical, inhalation or through sustained release.

A therapeutically-effective amount is at least the minimal dose, but less than a toxic dose, of an active agent which is necessary to impart therapeutic benefit to a human. Stated another way, a therapeutically-effective amount is an amount which induces, ameliorates or otherwise causes an improvement to reduce the alcohol intake, inhibit alcohol dependence, interference with the development of the dependence process and relapse prevention

‘Carriers’ as used herein include pharmaceutically-acceptable carriers, excipients, or stabilizers which are nontoxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecule weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG), and PLURONICS.

### BRIEF DESCRIPTION OF THE DRAWINGS

FIG. 1 shows effects of central ghrelin injection on alcohol intake (A) and alcohol preference (B) in mice. (Shown are the means±SEM of 8-10 animals).

FIG. 2 shows suppressed alcohol-induced locomotor activity in ghrelin receptor knockout mice (GHS-R −/−), compared to wild types (wt/wt) and heterozygotes (wt/−). ▪ Alcohol 1.0 g/kg i.p., □ vehicle. (Shown are the means±SEM of 6-13 animals).

FIG. 3 shows the absence of alcohol-induced dopamine release in the nucleus accumbens in ghrelin receptor knockout mice (GHS-R −/−) □, compared to wild types (wt/wt) Δ and heterozygotes (wt/−) ◯. (Shown are the means±SEM of 6-13 animals).

### DETAILED DESCRIPTION OF THE INVENTION

The present inventors have discovered that intraventricular administration of ghrelin increases both alcohol intake and alcohol preference in animal model (Example 4). Furthermore, they have discovered that unlike wildtype mice ghrelin receptor knockout mice do not show an alcohol-induced locomotor activity (Example 5). It is concluded that ghrelin signaling via its receptor (GHS-R) is required for alcohol to activate the mesolimbic dopamine system, and that compounds, ghrelin receptor ligands (GHS-RLs), interfering with this signaling can be used to treat alcohol related disorders, and other chemical substance addiction related disorders.

In one aspect the present invention provides a method for treating chemical substance addiction related disorders in humans comprising administering to a human in need thereof a therapeutically-effective amount of a compound which is a ghrelin receptor ligand (GHS-RL). The chemical substance addiction related disorder can be selected from, alcohol related disorders, cocaine addiction, amphetamine addiction, heroin addiction, cannabinoid addiction, and nicotine addiction. The ghrelin receptor ligand (GHS-RL) can be selected from a ghrelin receptor antagonist (GHS-RA), a ghrelin receptor inverse agonist (GHS-RIA) and a ghrelin receptor partial agonist (GHS-RPA).

In one preferred aspect the present invention provides a method for treating alcohol related disorders in humans comprising administering to a human in need thereof a therapeutically-effective amount of a compound which is a ghrelin receptor ligand (GHS-RL). The ghrelin receptor ligand (GHS-RL) can be selected from a ghrelin receptor antagonist (GHS-RA), a ghrelin receptor inverse agonist (GHS-RIA) and a ghrelin receptor partial agonist (GHS-RPA).

In another preferred aspect the present invention provides a method for treating alcohol addiction in humans comprising administering to a human in need thereof a therapeutically-effective amount of a compound which is a ghrelin receptor ligand (GHS-RL). The ghrelin receptor ligand (GHS-RL) can be selected from a ghrelin receptor antagonist (GHS-RA), a ghrelin receptor inverse agonist (GHS-RIA) and a ghrelin receptor partial agonist (GHS-RPA).

In another aspect the present invention provides a pharmaceutical composition comprising a ghrelin receptor ligand (GHS-RL) for the treatment of chemical substance addiction related disorders. The chemical substance addiction related disorder can be selected from, alcohol related disorders, cocaine addiction, amphetamine addiction, heroin addiction, cannabinoid addiction, and nicotine addiction. The ghrelin receptor ligand (GHS-RL) can be selected from a ghrelin receptor antagonist (GHS-RA), a ghrelin receptor inverse agonist (GHS-RIA) and a ghrelin receptor partial agonist (GHS-RPA).

In one preferred aspect the present invention provides a pharmaceutical composition comprising a ghrelin receptor ligand (GHS-RL) for the treatment of alcohol related disorders. The ghrelin receptor ligand (GHS-RL) can be selected from a ghrelin receptor antagonist (GHS-RA), a ghrelin receptor inverse agonist (GHS-RIA) and a ghrelin receptor partial agonist (GHS-RPA).

In one preferred aspect the present invention provides a pharmaceutical composition comprising a ghrelin receptor ligand (GHS-RL) for the treatment of alcohol addiction. The ghrelin receptor ligand (GHS-RL) can be selected from a ghrelin receptor antagonist (GHS-RA), a ghrelin receptor inverse agonist (GHS-RIA) and a ghrelin receptor partial agonist (GHS-RPA).

In another aspect the present invention provides use of a ghrelin receptor ligand (GHS-RL) in the manufacture of a medicament for the treatment of chemical substance addiction related disorders. The chemical substance addiction related disorder can be selected from, but is not limited to, alcohol related disorders, cocaine addiction, amphetamine addiction, heroin addiction, cannabinoid addiction, and nicotine addiction. The ghrelin receptor ligand (GHS-RL) can be selected from a ghrelin receptor antagonist (GHS-RA), a ghrelin receptor inverse agonist (GHS-RIA) and a ghrelin receptor partial agonist (GHS-RPA).

In one preferred aspect the present invention provides use of a ghrelin receptor ligand (GHS-RL) in the manufacture of a medicament for the treatment of alcohol related disorders. The ghrelin receptor ligand (GHS-RL) can be selected from a ghrelin receptor antagonist (GHS-RA), a ghrelin receptor inverse agonist (GHS-RIA) and a ghrelin receptor partial agonist (GHS-RPA).

In one preferred aspect the present invention provides use of a ghrelin receptor ligand (GHS-RL) in the manufacture of a medicament for the treatment of alcohol addiction. The ghrelin receptor ligand (GHS-RL) can be selected from a ghrelin receptor antagonist (GHS-RA), a ghrelin receptor inverse agonist (GHS-RIA) and a ghrelin receptor partial agonist (GHS-RPA).

In yet another aspect the present invention provides a method for the identification of a compound suitable for the treatment of chemical substance addiction related disorders, said method comprising the steps;
a) providing a test compound;
b) contacting said test compound with a ghrelin receptor;
c) determining the IC50 for inverse agonism, the IC50 for partial agonism and/or the IC50 for antagonism of said test compound for the ghrelin receptor;
d) comparing said IC50 for inverse agonism, IC50 for partial agonism and/or IC50 for antagonism with the corresponding IC50 values for a known ligand of the ghrelin receptor; and
d) determining that said test compound is suitable for the treatment of chemical substance addiction related disorders.

The chemical substance addiction related disorder can be selected from, alcohol related disorders, alcohol addiction, cocaine addiction, amphetamine addiction, heroin addiction, cannabinoid addiction, and nicotine addiction.

The ghrelin receptor used in the methods according to the invention can be the human ghrelin receptor (SwissProt entry Q92847), any orthologue thereof such as a non-human ghrelin receptor such as the murine (SwissProt entry Q99P50), the rat (SwissProt entry O08725), the rabbit (SwissProt entry A5A4K9), the pig (SwissProt entry Q95254), and a primate ghrelin receptor, and any genetic or allelic variants thereof.

Preferably the ghrelin receptor is the human ghrelin receptor, or a variant thereof such as a polypeptide having an amino acid sequence which has a sequence identity of more than 80%, such as more than 85%, preferably more than 90%, or eve more preferably more than 95%, compared to sequence of the human ghrelin receptor SwissProt entry Q92847, including a fragment of such a polypeptide able to bind ghrelin, or a polypeptide comprising such a fragment, such as a fusion protein.

The percent identity between two amino acid sequences is determined as follows. First, an amino acid sequence is compared to, for example, SwissProt entry Q92847 using the BLAST 2 Sequences (B12seq) program from the stand-alone version of BLASTZ containing BLASTN version 2.0.14 and BLASTP version 2.0.14. This stand-alone version of BLASTZ can be obtained from the U.S. government's National Center for Biotechnology Information web site at ncbi.nlm.nih.gov. Instructions explaining how to use the B12seq program can be found in the readme file accompanying BLASTZ. B12seq performs a comparison between two amino acid sequenced using the BLASTP algorithm. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq c:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide or amino acid residue is presented in both sequences. The percent identity is determined by dividing the number of matches by the length of the sequence set forth in an identified sequence followed by multiplying the resulting value by 100. For example, if a sequence is compared to the sequence set forth in SwissProt entry Q92847 (the length of this sequence 366) and the number of matches is 359, then the sequence has a percent identity of 98 (i.e., 360÷366*100=98.087) to the sequence according to SwissProt entry Q92847.

A ghrelin receptor ligand (GHS-RL) that can be used according to the present invention preferably has an IC50 for competitive binding with ghrelin which is less than 100 nM, more preferably less than 30 nM, and even more preferably less than 10 nM. The IC50 for competitive binding for a potential ghrelin receptor ligand (GHS-RL) according to the present invention can be determined as described in Example 2.

A ghrelin receptor antagonist (GHS-RA) that can be used according to the present invention preferably has an IC50 for antagonism which is less than 100 nM, more preferably less than 30 nM, and even more preferably less than 10 nM. The IC50 for antagonism for a potential ghrelin receptor antagonist (GHS-RA) according to the present invention can be determined as described in Example 2.

A ghrelin receptor inverse agonist (GHS-RIA) that can be used according to the present invention preferably has an IC50 for inverse agonism which is less than 300 nM, more preferably less than 100 nM, and even more preferably less than 30 nM. The 1050 for inverse agonism for a potential ghrelin receptor inverse agonist (GHS-RIA) according to the present invention can be determined as described in Example 2.

A ghrelin receptor inverse agonist (GHS-RIA) that can be used according to the present invention preferably has an 1050 for antagonism which is higher than 100 nM, preferably higher than 300 nM, and even more preferably higher than 1 μM. The IC50 for antagonism for a potential ghrelin receptor inverse agonist (GHS-RIA) according to the present invention can be determined as described in Example 2.

The ratio of the IC50 for inverse agonism and the IC50 for antagonism of the ghrelin receptor inverse agonist (GHS-RIA) that can be used according to the present invention preferably is in the range 1:1000 to 1:10, preferably in the ratio 1:200 to 1:50.

A the ghrelin receptor partial agonist (GHS-RPA) that can be used according to the present invention preferably has an IC50 for partial agonism which is less than 300 nM, more preferably less than 100 nM, and even more preferably less than 30 nM. The IC50 for partial agonism for a potential ghrelin receptor partial agonist (GHS-RPA) according to the present invention can be determined as described in Example 1

A ghrelin receptor partial agonist (GHS-RPA) that can be used according to the present invention preferably has an IC50 for antagonism which is higher than 100 nM, preferably higher than 300 nM, and even more preferably higher than 1 μM.

The ratio of the IC50 for inverse agonism and the IC50 for antagonism of the ghrelin receptor partial agonist (GHS-RPA) that can be used according to the present invention preferably is in the range 1:1000 to 1:10, preferably in the ratio 1:200 to 1:50.

The maximum response of the ghrelin receptor partial agonist (GHS-RPA) that can be used according to the present invention preferably is less than 95% of the response obtained with 10 μM ghrelin, such less than 90%, less than 80%, less than 70%, less than 60%, or less than 50% of the response obtained with 10 μM ghrelin, and even more preferably less than 40% of the response obtained with 10 μM ghrelin, such as less than 30% or less than 20%.

The GHS-RLs, (GHS-RAs, GHS-RIA and GHS-RPAs) useful in the presently claimed methods include but are not limited to natural products, synthetic organic compounds, peptides, proteins, antibodies, antibody fragments, single chain antibodies, and antibody based constructs. The current level of skill in the art of receptor binding and ghrelin receptor assays places GHS-RLs well within the grasp of the ordinarily skilled artisan. There are several routine approaches for identifying a GHS-RL. One basic scheme involves a receptor competitive binding assay according to Example 1. In this scheme, the GHS-RL test compound is first checked to determine if it binds GHS-R. This is accomplished using routine radiometric binding methods.

Assays for GHS-R antagonism and agonism include second messenger reporter assays such as inositol phosphate accumulation, as described in Example 2, and calcium flux, as well as CRE and NFAT reporter assay as described in Example 3.

Bioassays for GHS-R antagonism and agonism include suppression of ghrelin-induced Fos induction in the arcuate nucleus or suppression of ghrelin-induced food intake.

Bioassays for determining the effect of the ghrelin receptor ligands (GHS-RLs) according to the present invention on alcohol intake, alcohol-induced increased locomotor activity, dopamine release, and condition place preference (CPP) are outlined in Examples 5, 6 and 7.

Compounds that can be used according to the present invention can be selected from, but are not limited to,

compounds having the formula (A)

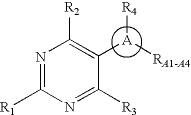

wherein R₁ is a member selected from the group consisting of hydrogen, alkyl, alkoxy, aryl, arylalkyl, cyano, cycloalkyl, cycloalkylalkyl, haloalkoxy, haloalkyl, halogen, heteroaryl, heteroarylalkyl, heterocycle, heterocyclealkyl, hydroxy, mercapto, nitro, and —NR_{A}R_{B};
R_{A} and R_{B} are each independently a member selected from the group consisting of hydrogen, alkoxycarbonyl, alkyl, alkylcarbonyl, alkoxysulfonyl, alkylsulfonyl, aryl, arylalkyl, and formyl;
R₂ is a member selected from the group consisting of hydrogen, alkyl, alkoxy, alkoxycarbonyl, aryl, arylalkyl, cyano, cycloalkyl, cycloalkylalkyl, haloalkoxy, haloalkyl, halogen, heteroaryl, heteroarylalkyl, heterocycle, heterocyclealkyl, hydroxy, mercapto, nitro, —NR_{C}R_{D}, and (NR_{C}R_{D})alkyl;
R_{C} and R_{D} are each independently a member selected from the group consisting of hydrogen, alkoxycarbonyl, alkenyl, alkyl, alkylcarbonyl, alkoxysulfonyl, alkylsulfonyl, aryl, arylalkyl, cycloalkyl, cycloalkylalkyl, formyl, and hydroxyalkyl;
R₃ is a member selected from the group consisting of alkenyl, alkenylalkoxyalkyl, alkenyloxy, alkenyloxyalkyl, alkoxy, alkoxyalkoxy, alkoxyalkoxyalkyl, alkoxyalkyl, alkoxycarbony, alkoxycarbonylalkyl, alkoxysulfonyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynylalkoxyalkyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, haloalkoxy, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyallyl, heterocyclethio, heterocyclethioalkyl, hydroxy, hydroxyalkyl, mercapto, nitro, —NR_{E}R_{H}, (NR_{E}R_{H})alkyl, (NR_{E}R_{F})carbonylalkenyl, (NR_{E}R_{F})carbonylalkyl, (NR_{E}R_{F})sulfonyl, and (NR_{E}R_{F})sulfonylalkyl;
R_{E} and R_{F} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, heterocyclecarbonyl, (NZ₁Z₂)alkyl, and (NZ₁Z₂)carbonyl;
Z₁ and Z₂ are each independently a member selected from the group consisting of hydrogen, alkoxycarbonyl, alkyl, alkylcarbonyl, alkoxysulfonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
R₄ is a member selected from the group consisting of alkenyl, alkenyloxy, alkenyloxyalkyl, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, alkynyloxy, alkynyloxyalkyl, aryl, arylalkoxy, arylalkoxyalkyl, arylalkyl, arylalkylthio, arylalkylthioalkyl, aryloxy, aryloxyalkyl, arylthio, arylthioalkyl, carboxy, carboxyalkyl, cyano, cyanoalkyl, cycloalkenyl, cycloalkenylalkoxy, cycloalkenylalkoxyalkyl, cycloalkenylalkyl, cycloalkenylalkylthio, cycloalkenylalkylthioalkyl, cycloalkenyloxy, cycloalkenyloxyalkyl, cycloalkenylthio, cycloalkenylthioalkyl, cycloalkyl, cycloalkylalkoxy, cycloalkylalkoxyalkyl, cycloalkylalkyl, cycloalkylalkylthio, cycloalkylalkylthioalkyl, cycloalkyloxy, cycloalkyloxyalkyl, cycloalkylthio, cycloalkylthioalkyl, formyl, formylalkyl, haloalkoxy, haloalkyl, halogen, heteroaryl, heteroarylalkoxy, heteroarylalkoxyalkyl, heteroarylalkyl, heteroarylalkylthio, heteroarylalkylthioalkyl, heteroaryloxy, heteroaryloxyalkyl, heteroarylthio, heteroarylthioalkyl, heterocycle, heterocyclealkoxy, heterocyclealkoxyalkyl, heterocyclealkyl, heterocyclealkylthio, heterocyclealkylthioalkyl, heterocycleoxy, heterocycleoxyalkyl, heterocyclethio, heterocyclethioalkyl, hydroxy, hydroxyalkyl, mercapto, nitro, —NR_{G}R_{H}, (NR_{G}R_{H})alkyl, (NR_{G}R_{H})carbonyl, and (NR_{G}R_{H})sulfonyl;
R_{G} and R_{H} are each independently a member selected from the group consisting of hydrogen, alkoxyalkyl, alkoxyalkylcarbonyl, alkoxycarbonyl, alkoxysulfonyl, alkyl, alkylcarbonyl, alkylsulfonyl, alkylthioalkyl, alkylthioalkylcarbonyl, alkylthiocarbonyl, aryl, arylalkoxyalkyl, arylalkyl, arylcarbonyl, cycloalkyl, cycloalkylalkoxyalkyl, cycloalkylalkyl, cycloalkylcarbonyl, formyl, heteroaryl, heteroarylalkoxyalkyl, heteroarylalkyl, heteroarylcarbonyl, heterocycle, heterocyclealkoxyalkyl, heterocyclealkyl, heterocyclecarbonyl, (NZ₃Z₄)alkyl, and (NZ₃Z₄)carbonyl;
Z₃ and Z₄ are each independently a member selected from the group consisting of hydrogen, alkoxycarbonyl, alkoxysulfonyl, alkyl, alkylcarbonyl, alkylsulfonyl, aryl, arylalkyl, arylcarbonyl, cycloalkyl, cycloalkylalkyl, cycloalkylcarbonyl, formyl, heteroaryl, heteroarylalkyl, heteroarylcarbonyl, heterocycle, heterocyclealkyl, and heterocyclecarbonyl;
A is a member selected from the group consisting of aryl, cycloalkyl, cycloalkenyl, heteroaryl, and heterocycle;
R_{A1}, R_{A2}, R_{A3}, and R_{A4} are each independently a member selected from the group consisting of hydrogen, alkenyl, alkenyloxy, alkoxy, alkoxyalkoxy, alkoxyalkoxyalkyl, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, aryl, carboxy, carboxyalkyl, cyano, cyanoalkyl, cycloalkyl, formyl, haloalkoxy, haloalkyl, halogen, heteroaryl, heterocycle, hydroxy, hydroxyalkyl, mercapto, nitro, —NR_{J}R_{K}, (NR_{J}R_{K})alkyl, (NR_{J}R_{K})carbonyl, and (NR_{J}R_{K})sulfonyl; and
R_{J} and R_{K} are each independently a member selected from the group consisting of hydrogen, alkoxycarbonyl, alkyl, alkylcarbonyl, alkoxysulfonyl, alkylsulfonyl, aryl, arylalkyl, and formyl;
   Specific compounds of formula (A) include, but are not limited to:
6-[(Benzyloxy)methyl]-5-{4-[(4-chlorobenzyl)amino]phenyl}pyrimidine-2,4-diamine;
5-{4-[(4-Chlorobenzyl)amino]phenyl}-6-(methoxymethyl)pyrimidine-2,4-diamine;
5-{4-[(4-Chlorobenzyl)amino]phenyl}-6-{[(2-fluoro-3-methylbenzyl)oxy]methyl}pyrimidine-2,4-diamine;
5-{4-[(4-Chlorobenzyl)amino]phenyl}-6-(3-phenylpropyl)pyrimidine-2,4-diamine;
5-{4-[(4-Chlorobenzyl)amino]phenyl}-6-(phenoxymethyl)pyrimidine-2,4-diamine;
5-{4-[(4-Chlorobenzyl)amino]phenyl}-6-{[(3-methylbenzyl)oxy]methyl}pyrimidine-2,4-diamine;
5-{4-[(4-Chlorobenzyl)amino]phenyl}-6-{[(2-methoxybenzyl)oxy]methyl}pyrimidine-2,4-diamine;
5-{4-[(4-Chlorobenzyl)amino]phenyl}-6-{[(3-methoxybenzyl)oxy]methyl}pyrimidine-2,4-diamine;
5-{4-[(4-Chlorobenzyl)amino]phenyl}-6-{[(4-methoxybenzyl)oxy]methyl}pyrimidine-2,4-diamine;
5-{4-[(4-Chlorobenzyl)amino]phenyl}-6-{[(2-fluorobenzyl)oxy]methyl}pyrimidine-2,4-diamine;
5-{4-[(4-Chlorobenzyl)amino]phenyl}-6-{[(4-fluorobenzyl)oxy]methyl}pyrimidine-2,4-diamine;
5-{4-[(4-Chlorobenzyl)amino]phenyl}-6-{[(2-chlorobenzyl)oxy]methyl}pyrimidine-2,4-diamine;
5-{4-[(4-Chlorobenzyl)amino]phenyl}-6-{[(4-chlorobenzyl)oxy]methyl}pyrimidine-2,4-diamine;
6-{[(2-Bromobenzyl)oxy]methyl}-5-{4-[(4-chlorobenzyl)amino]phenyl}pyrimidine-2,4-diamine;
5-{4-[(4-Chlorobenzyl)amino]phenyl}-6-({[3-(trifluoromethyl)benzyl]oxy}methyl)pyrimidine-2,4-diamine;
5-{4-[(4-Chlorobenzyl)amino]phenyl}-6-({[4-(methylthio)benzyl]oxy}methyl)pyrimidine-2,4-diamine;
5-{4-[(4-Chlorobenzyl)amino]phenyl}-6-{[(2,4-dimethylbenzyl)oxy]methyl}pyrimidine-2,4-diamine;
5-{4-[(4-Chlorobenzyl)amino]phenyl}-6-{[(3,5-dimethylbenzyl)oxy]methyl}pyrimidine-2,4-diamine;
5-{4-[(4-Chlorobenzyl)amino]phenyl}-6-{[(2,3-dichlorobenzyl)oxy]methyl}pyrimidine-2,4-diamine;
5-{4-[(4-Chlorobenzyl)amino]phenyl}-6-{[(2,5-dichlorobenzyl)oxy]methyl}pyrimidine-2,4-diamine;
5-{4-[(1,3-Benzodioxol-4-ylmethyl)amino]phenyl}-6-[(benzyloxy)methyl]pyrimidine-2,4-diamine;
tert-butyl 2-[(4-{2,4-Diamino-6-[(benzyloxy)methyl]pyrimidin-5-yl}phenyl)amino]ethylcarbamate;
6-[(Benzyloxy)methyl]-5-{4-[(3-furylmethyl)amino]phenyl}pyrimidine-2,4-diamine;
6-[(Benzyloxy)methyl]-5-{4-[(tetrahydrofuran-3-ylmethyl)amino]phenyl}pyrimidine-2,4-diamine;
4-Chloro-N-(4-{2,4-diamino-6-[(benzyloxy)methyl]pyrimidin-5-yl}phenyl)benzamide;
6-[(Benzyloxy)methyl]-5-{4-[(pyridin-2-ylmethyl)amino]phenyl}pyrimidine-2,4-diamine;
6-[(Benzyloxy)methyl]-5-{4-[(pyridin-3-ylmethyl)amino]phenyl}pyrimidine-2,4-diamine;
6-[(Benzyloxy)methyl]-5-{4-[(1H-imidazol-4-ylmethyl)amino]phenyl}pyrimidine-2,4-diamine;
6-[(Benzyloxy)methyl]-5-[4-(dimethylamino)phenyl]pyrimidine-2,4-diamine;
6-[(Benzyloxy)methyl]-5-[4-(methylamino)phenyl]pyrimidine-2,4-diamine;
6-[(Benzyloxy)methyl]-5-[4-(ethylamino)phenyl]pyrimidine-2,4-diamine;
6-[(Benzyloxy)methyl]-5-[4-(propylamino)phenyl]pyrimidine-2,4-diamine;
6-[(Benzyloxy)methyl]-5-[4-(isobutylamino)phenyl]pyrimidine-2,4-diamine;
6-[(Benzyloxy)methyl]-5-[4-(neopentylamino)phenyl]pyrimidine-2,4-diamine;
6-[(Benzyloxy)methyl]-5-{4-[(cyclopropylmethyl)amino]phenyl}pyrimidine-2,4-diamine;
2-Butoxy-N-(4-{2,4-diamino-6-[(benzyloxy)methyl]pyrimidin-5-yl}phenyl)acetamide;
5-{4-[(4-Chlorobenzyl)amino]phenyl}-6-tetrahydrofuran-2-ylpyrimidine-2,4-diamine;
6-[(2-Butoxyethoxy)methyl]-5-{4-[(4-chlorobenzyl)amino]phenyl}pyrimidine-2,4-diamine;
6-[(Benzyloxy)methyl]-5-{4-[(1-ethylpropyl)amino]phenyl}pyrimidine-2,4-diamine;
4-{[(4-{2,4-Diamino-6-[(benzyloxy)methyl]pyrimidin-5-yl}phenyl)amino]methyl}benzonitrile;
4-{[(4-{2,4-Diamino-6-[(benzyloxy)methyl]pyrimidin-5-yl}phenyl)(methyl)amino]methyl}benzonitrile;
5-{4-[(4-Chlorobenzyl)amino]phenyl}-6-[(3-methylbutoxy)methyl]pyrimidine-2,4-diamine;
N-(4-{2,4-Diamino-6-[(benzyloxy)methyl]pyrimidin-5-yl}phenyl)propanamide;
6-[(Benzyloxy)methyl]-5-{4-[(pyridin-4-ylmethyl)amino]phenyl}pyrimidine-2,4-diamine;
N-(4-Chlorobenzyl)-N-(4-{2,4-diamino-6-[(benzyloxy)methyl]pyrimidin-5-yl}phenyl)acetamide;
4-Chlorobenzyl(4-{2,4-diamino-6-[(benzyloxy)methyl]pyrimidin-5-yl}phenyl)formamide;
6-[(Benzyloxy)methyl]-5-{4-[(1H-imidazol-2-ylmethyl)amino]phenyl}pyrimidine-2,4-diamine;
6-[(Benzyloxy)methyl]-5-(4-{[(6-chloropyridin-3-yl)methyl]amino}phenyl)pyrimidine-2,4-diamine;
N-benzyl-3-(2,6-diamino-5-{4-[(4-chlorobenzyl)amino]phenyl}pyrimidin-4-yl)propanamide;
3-(2,6-Diamino-5-{4-[(4-chlorobenzyl)amino]phenyl}pyrimidin-4-yl)-N-phenylpropanamide;
6-[(Benzyloxy)methyl]-5-{4-[(1-pyridin-4-ylethyl)amino]phenyl}pyrimidine-2,4-diamine;
4-{1-[(4-{2,4-Diamino-6-[(benzyloxy)methyl]pyrimidin-5-yl}phenyl)amino]ethyl}benzonitrile;
6-[(Benzyloxy)methyl]-5-{4-[(4-methoxybenzyl)amino]phenyl}pyrimidine-2,4-diamine;
6-[(Benzyloxy)methyl]-5-(4-{[1-(4-chlorophenyl)ethyl]amino}phenyl)pyrimidine-2,4-diamine;
6-[(Benzyloxy)methyl]-5-{4-[(cyclohexylmethyl)amino]phenyl}pyrimidine-2,4-diamine;
N-butyl-3-(2,6-diamino-5-{4-[(4-chlorobenzyl)amino]phenyl}pyrimidin-4-yl)propanamide;
3-(2,6-Diamino-5-{4-[(4-chlorobenzyl)amino]phenyl}pyrimidin-4-yl)-N-(3-methylphenyl)propanamide;
6-[(Benzyloxy)methyl]-5-{4-[(4-chlorobenzyl)oxy]phenyl}pyrimidine-2,4-diamine;
6-[(Benzyloxy)methyl]-5-(4-{[(4-chlorobenzyl)amino]methyl}phenyl)pyrimidine-2,4-diamine;
5-[4-(Benzylamino)phenyl]-6-[(benzyloxy)methyl]pyrimidine-2,4-diamine;
6-[(Benzyloxy)methyl]-5-(4-{[(4-nitrophenyl)amino]methyl}phenyl)pyrimidine-2,4-diamine;
N-(4-{2,4-Diamino-6-[(benzyloxy)methyl]pyrimidin-5-yl}benzyl)-N′-propylurea;
4-{[(4-{2,4-Diamino-6-[(cyclobutylmethoxy)methyl]pyrimidin-5-yl}phenyl)amino]methyl}benzonitrile;
4-[(4-{2,4-Diamino-6-[(benzyloxy)methyl]pyrimidin-5-yl}phenoxy)methyl]benzonitrile;
5-{4-[(4-Chlorobenzyl)amino]phenyl}-6-[(tetrahydro-2H-pyran-2-ylmethoxy)methyl]pyrimidine-2,4-diamine;
6-[(Benzyloxy)methyl]-5-[4-({[6-(trifluoromethyl)pyridin-3-yl]methyl}amino)phenyl]pyrimidine-2,4-diamine;
4-[(4-{2,4-Diamino-6-[(benzyloxy)methyl]pyrimidin-5-yl}benzyl)amino]benzonitrile;
3-[(4-{2,4-Diamino-6-[(benzyloxy)methyl]pyrimidin-5-yl}phenoxy)methyl]benzonitrile;
5-{[(4-{2,4-Diamino-6-[(benzyloxy)methyl]pyrimidin-5-yl}phenyl)amino]methyl}pyridine-2-carbonitrile;
6-[(Benzyloxy)methyl]-5-{4-[2-(4-chlorophenyl)ethoxy]phenyl}pyrimidine-2,4-diamine;
6-[(Benzyloxy)methyl]-5-[4-(pyridin-3-ylmethoxy)phenyl]pyrimidine-2,4-diamine;
6-[(Benzyloxy)methyl]-5-{4-[(tetrahydro-2H-pyran-4-ylmethyl)amino]phenyl}pyrimidine-2,4-diamine;
6-[(Benzyloxy)methyl]-5-(4-{[4-(trifluoromethoxy)benzyl]amino}phenyl)pyrimidine-2,4-diamine;
5-{4-[(4-Chlorobenzyl)amino]phenyl}-6-[(cyclohexylmethoxy)methyl]pyrimidine-2,4-diamine;
4-{[(4-{2,4-Diamino-6-[(benzyloxy)methyl]pyrimidin-5-yl}phenyl)amino]methyl}pyridine-2-carbonitrile;
6-[(4-{2,4-Diamino-6-[(benzyloxy)methyl]pyrimidin-5-yl}benzyl)amino]nicotinonitrile;
5-{4-[(4-Chlorobenzyl)amino]phenyl}-6-{[(3-chlorobenzyl)oxy]methyl}pyrimidine-2,4-diamine;
5-{4-[(4-Chlorobenzyl)amino]phenyl}-6-{[(2-methylbenzyl)oxy]methyl}pyrimidine-2,4-diamine;
6-[(Benzyloxy)methyl]-5-{4-[(4-nitrobenzyl)amino]phenyl}pyrimidine-2,4-diamine;
6-[(Benzyloxy)methyl]-5-(4-{[(2-chloropyridin-4-yl)methyl]amino}phenyl)pyrimidine-2,4-diamine;
6-[(Benzyloxy)methyl]-5-{4-[(pyrimidin-5-ylmethyl)amino]phenyl}pyrimidine-2,4-diamine;
6-[(Benzyloxy)methyl]-5-{4-[(thien-2-ylmethyl)amino]phenyl}pyrimidine-2,4-diamine;
6-[(Benzyloxy)methyl]-5-{4-[(thien-3-ylmethyl)amino]phenyl}pyrimidine-2,4-diamine;
6-[(Benzyloxy)methyl]-5-[4-({[1-(4-chlorophenyl)ethyl]amino}methyl)phenyl]pyrimidine-2,4-diamine;
6-[(Benzyloxy)methyl]-5-(4-{[2-(4-nitrophenyl)ethyl]amino}phenyl)pyrimidine-2,4-diamine;
6-[(Benzyloxy)methyl]-5-(4-{[2-(4-chlorophenyl)ethyl]amino}phenyl)pyrimidine-2,4-diamine;
6-[(Benzyloxy)methyl]-5-{4-[(cycloheptylamino)methyl]phenyl}pyrimidine-2,4-diamine;
6-Benzyloxymethyl-5-[4-(pyridin-4-ylmethoxy)-phenyl]-pyrimidine-2,4-diamine.
5-{4-[(4-Chlorobenzyl)amino]phenyl}-6-ethylpyrimidine-2,4-diamine;
4-({[4-(2,4-Diamino-6-ethylpyrimidin-5-yl)phenyl]amino}methyl)benzonitrile;
5-{4-[(3,4-Dichlorobenzyl)amino]phenyl}-6-ethylpyrimidine-2,4-diamine;
5-{4-[(4-Chlorobenzyl)amino]phenyl}-6-propylpyrimidine-2,4-diamine;
5-(4-{[2-(Benzyloxy)ethyl]amino}phenyl)-6-ethylpyrimidine-2,4-diamine; and
6-Ethyl-5-{4-[(4-nitrobenzyl)amino]phenyl}pyrimidine-2,4-diamine;
   compounds having the formula (B)

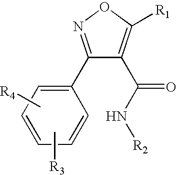

wherein R₁ is a member selected from the group consisting of alkoxyalkyl, alkyl, alkylC(O)NHalkyl, alkylS(O)₂NHalkyl, alkenyl, aryl, arylalkyl, heterocycle, heterocyclealkyl, hydroxyalkyl, RₐR_{b}N—, RₐR_{b}Nalkyl, RₐR_{b}Ncarboxyalkyl, wherein the alkyl group of said arylalkyl and the alkyl group of said heterocyclealkyl may be substituted with 0, 1 or 2 groups that are a member selected from the group consisting of halogen and hydroxy;
R₂ is a member selected from the group consisting of alkyl, aryl, arylalkyl, cycloalkyl, cycloalkylalkyl, cycloalkenyl, cycloalkenylalkyl;
R₃ and R₄ are each members independently selected from the group consisting of hydrogen, alkyl, alkoxy, aryl, halogen, haloalkyl, cycloalkyl, cyano and nitro;
Rₐ and R_{b} are each members independently selected from the group consisting of hydrogen, alkoxyalkyl, alkyl, alkylcarbonyl, alkylsulfonyl, aryloxyalkyl and R_{c}R_{d}Ncarboxyalkylcarbonyl; and
R_{c} and R_{d} are each members independently selected from the group consisting of hydrogen, and alkyl;
   specific compounds of formula (B) include, but are not limited to:
3-(2,6-dichlorophenyl)-N-[4-(diethylamino)phenyl]-5-methylisoxazole-4-carboxamide;
3-(2-chloro-6-fluorophenyl)-N-[4-(diethylamino)phenyl]-5-methylisoxazole-4-carboxamide;
5-but-3-enyl-3-(2,6-dichlorophenyl)-N-[4-(diethylamino)phenyl]isoxazole-4-carboxamide;
3-(2,6-dichlorophenyl)-N-[4-(diethylamino)phenyl]-5-(3,4-dihydroxybutyl)isoxazole-4-carboxamide;
3-(2,6-dichlorophenyl)-N-[4-(diethylamino)phenyl]-5-ethylisoxazole-4-carboxamide;
3-(2-chloro-6-nitrophenyl)-N-[4-(diethylamino)phenyl]-5-methylisoxazole-4-carboxamide;
3-(2,6-dichlorophenyl)-N-[4-(diethylamino)phenyl]-5-(4-hydroxybutyl)isoxazole-4-carboxamide;
3-(2,6-dichlorophenyl)-N-[4-(diethylamino)-2-methylphenyl]-5-methylisoxazole-4-carboxamide;
N-[4-(diethylamino)phenyl]-5-methyl-3-(2-nitrophenyl)isoxazole-4-carboxamide;
3-(2,6-dichlorophenyl)-N-{4-[ethyl(isopropyl)amino)phenyl}-5-methylisoxazole-4-carboxamide;
5-(4-aminobutyl)-3-(2,6-dichlorophenyl)-N14-(diethylamino)phenyl]isoxazole-4-carboxamide;
3-(2-bromophenyl)-N-[4-(diethylamino)phenyl]-5-methylisoxazole-4-carboxamide;
3-(2,6-dichlorophenyl)-N-[4-(diethylamino)phenyl]-5-propylisoxazole-4-carboxamide;
3-(2,6-dichlorophenyl)-N-[4-(diethylamino)phenyl]-5-isopropylisoxazole-4-carboxamide;
3-(2,6-dichlorophenyl)-N-[4-(diethylamino)phenyl]-5-(2-furyl)isoxazole-4-carboxamide;
3-(2,6-dichlorophenyl)-N-[4-(diethylamino)-2-methoxyphenyl]-5-methylisoxazole-4-carboxamide;
N-{4-[tert-butyl(ethyl)amino]phenyl}-3-(2,6-dichlorophenyl)-5-methylisoxazole-4-carboxamide;
3-(2,6-dichlorophenyl)-N-[4-(diethylamino)-2-hydroxyphenyl]-5-methylisoxazole-4-carboxamide;
N-{4-[(2-chloroethyl)(ethyl)amino]phenyl}dichlorophenyl)-5-methylisoxazole-4-carboxamide;
3-(2,6-dichlorophenyl)-N-{4-[ethyl(propyl)amino]phenyl}-5-methylisoxazole-4-carboxamide;
N-{4-[butyl(ethyl)amino]phenyl}(2,6-dichlorophenyl)-5-methylisoxazole-4-carboxamide;
3-(2,6-dichlorophenyl)-N-[4-(diethylamino)-2-methylphenyl]-5-propylisoxazole-4-carboxamide;
3-(2,6-dichlorophenyl)-N-[4-(diethylamino)-2-(piperidin-1-ylcarbonyl)phenyl]-5-methylisoxazole-4-carb oxamide;
[3-(2,6-dichlorophenyl)-N-[4-(diethylamino)phenyl]-5-(2-phenylethyl)isoxazole-4-carboxamide;
3-(2,6-dichlorophenyl)-N-[4-(diethylamino)-2-ethylphenyl]-5-methylisoxazole-4-carboxamide;
3-(2,6-dichlorophenyl)-N-[4-(diethylamino)phenyl]-5-[3-(dimethylamino)-3-oxopropyl]isoxazole-4-carboxamide;
3-(2,6-dichlorophenyl)-N-[4-(diethylamino)phenyl]-5-[2-(1,3-dioxan-2-yl)ethyl]isoxazole-4-carboxamide;
5-[4-(acetylamino)butyl]-3-(2,6-dichlorophenyl)-N-[4-(diethylamino)phenyl]isoxazole-4-carboxamide;
3-(2,6-dichlorophenyl)-N-[4-(diethylamino)phenyl]-5-{4-[(methylsulfonyl)amino]butyl}isoxazole-4-carboxamide;
3-(2,6-dichlorophenyl)-N-[4-(diethylamino)phenyl]-5-[3-(1,3-dioxan-2-yl)propyl]isoxazole-4-carboxamide;
3-(2,6-dichlorophenyl)-N-[4-(diethylamino)phenyl]-5-(3-hydroxypropyl)isoxazole-4-carboxamide;
3-(2,6-dichlorophenyl)-N-[4-(diethylamino)phenyl]-5-(2-hydroxy-2-phenylethyl)isoxazole-4-carboxamide;
3-(2,6-dichlorophenyl)-N-[4-(diethylamino)phenyl]-5-(2-tetrahydro-2H-pyran-2-ylethyl)isoxazole-4-carboxamide;
3-(2,6-dichlorophenyl)-N-[4-(diethylamino)phenyl]-5-(tetrahydro-2H-pyran-4-ylmethyl)isoxazole-4-carboxamide;
5-butyl-3-(2,6-dichlorophenyl)-N-[4-(diethylamino)phenyl]isoxazole-4-carboxamide;
3-(2,6-dichlorophenyl)-5-[2-(1,3-dioxan-2-yl)ethyl]-N-{2-[(2-methyl-1,2,3,4-tetrahydroisoquinolin-1-yl)methyl]phenyl}isoxazole-4-carboxamide;
3-(2,6-dichlorophenyl)-N-[4-(diethylamino)phenyl]-5-[2-oxopyrrolidin-1-yl)methyl]isoxazole-4-carboxamide;
N-(2-{[(1,3-benzodioxol-5-ylmethyl)amino]carbonyl}phenyl)-3-(2,6-dichlorophenyl)-5-methylisoxazole-4-carboxamide;
3-(2,6-dichlorophenyl)-N-[4-(diethylamino)phenyl]-5-[2-(2-oxopyrrolidin-1-yl)ethyl]isoxazole-4-carboxamide;
3-(2,6-dichlorophenyl)-N-[4-(diethylamino)phenyl]-5-[2-(3-methyl-2-oxoimidazolidin-1-yl)ethyl]isoxazole-4-carboxamide;
3-(2,6-dichlorophenyl)-N-[4-(diethylamino)phenyl]-5-[2-(dimethylamino)-2-oxoethyl]isoxazole-4-carboxamide;
3-(2,6-dichlorophenyl)-N-[4-(diethylamino)phenyl]-5-[2-(3,3-dimethyl-2-oxopyrrolidin-1-yl)ethyl]isoxazole-4-carboxamide;
3-(2,6-dichlorophenyl)-N-[4-(diethylamino)cyclohexyl]-5-methylisoxazole-4-carboxamide;
3-(2,6-dichlorophenyl)-N-[4-(diethylamino)-2-methylphenyl]-5-[2-(1,3-dioxan-2-yl)ethyl]isoxazole-4-carboxamide;
3-(2,6-dichlorophenyl)-N-[4-(diethylamino)phenyl]-5-[2-(1,3-dioxolan-2-yl)ethyl]isoxazole-4-carboxamide;
3-(2,6-dichlorophenyl)-N-[4-(diethylamino)phenyl]-5-(2-methoxyethyl)isoxazole-4-carboxamide;
3-(2,6-dichlorophenyl)-N-[4-(diethylamino)phenyl]-5-(2-tetrahydrofuran-2-ylethyl) isoxazole-4-carboxamide;
3-(2,6-dichlorophenyl)-N-[2-(3,4-dihydroisoquinolin-2(1H)-ylcarbonyl)phenyl]-5-methylisoxazole-4-carboxamide;
3-(2,6-dichlorophenyl)-N-(2-{[(2,3-dihydro-1-benzofuran-5-ylmethyl)amino]carbonyl}phenyl)-5-methylisoxazole-4-carboxamide;
3-(2,6-dichlorophenyl)-N-{(1R)-1-[4-(diethylamino)phenyl]ethyl}-5-methylisoxazole-4-carboxamide;
3-(2,6-dichlorophenyl)-N-[4-(diethylamino)phenyl]-5-[2-(2-oxopiperidin-1-yl)ethyl]isoxazole-4-carboxamide;
5-{2-[acetyl(methyl)amino]ethyl}-3-(2,6-dichlorophenyl)-N-[4-(diethylamino)phenyl]isoxazole-4-carboxamide;
3-(2,6-dichlorophenyl)-N-(4-{2-[(3-ethoxypropyl)amino]ethyl}phenyl)-5-methylisoxazole-4-carboxamide;
3-(2,6-dichlorophenyl)-N-(4-{2-[(3-isopropoxypropyl)amino]ethyl}phenyl)-5-methylisoxazole-4-carboxamide; and
3-(2,6-dichlorophenyl)-5-methyl-N-(4-{2-[(2-phenoxyethyl)amino]ethyl}phenyl)isoxazole-4-carboxamide.
   compounds having the formula (C)

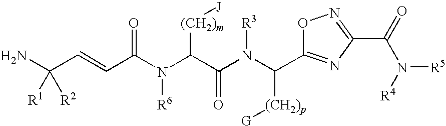

wherein R¹ and R² independently of each other are hydrogen or C1-6alkyl, or R¹ and R² taken together form a C2-5alkylene group;
J is a group

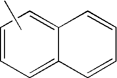

optionally substituted with one or more C1-6alkyl or halogen;
m is 2 or 3; R³ is C1-6alkyl; p is 1, 2 or 3;
G is a group

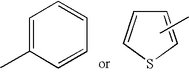

optionally substituted with one or more C1-6alkyl or halogen;
R⁴ and R⁵ independently of each other are hydrogen or C1-6alkyl; and
R⁶ is hydrogen or C1-6alkyl, preferably hydrogen.

Specific compounds of formula (C) include, but are not limited to:
(2E)-4-Amino-4-methylpent-2-enoic acid{(R)-1-[N-[1-(3-(N-methylcarbamoyl)-1,2,4-oxadiazol-5-yl)-2-phenylethyl]-N-methylcarbamoyl]-2-(2-naphthyl)ethyl}amide:
(2E)-4-amino-4-methylpent-2-enoic acid {1-[N-[1-(3-(N,N-dimethylcarbamoyl)-1,2,4-oxadiazole-5-yl)-2-phenylethyl]-N-methylcarbamoyl]-2-(2-naphthyl)ethyl}amide
(2E)-4-amino-4-methylpent-2-enoic acidN-{(R)-1-[N-[1-(3-(N,N-di-methylcarbamoyl)-1,2,4-oxadiazole-5-yl)-2-phenylethyl]-N-methylcarbamoyl]-2-(2-naphthyl)-ethyl}-N-methylamide
(R,E)-5-(1-(2-(4-amino-N,4-dimethylpent-2-enamido)-N-methyl-3-(naphthalen-2-yl)propanamido)-2-phenylethyl)-N,N-dimethyl-1,2,4-oxadiazole-3-carboxamide
   compounds having the formula (D)

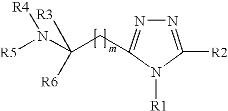

wherein R1 and R2 are independently of one another selected from the group consisting of “hydrogen atom, alkyl, alkenyl, alkynyl, cycloalkyl, cycloalkylalkyl, aryl, heteroaryl, arylalkyl, heteroarylalkyl, heterocyclyl, heterocyclylalkyl, alkylsulfo[pi]yl, arylsulfonyl, arylalkylsulfonyl” which are optionally substituted in the alkyl, cycloalkyl, cycloalkylalkyl, aryl, heteroaryl, arylalkyl, heteroarylalkyl, heterocyclyl and/or heterocyclylalkyl group by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”; and
preferably are selected from the group consisting of “alkyl, aryl, heteroaryl, arylalkyl, heteroarylalkyl” optionally being substituted by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”;
one of radicals R3 and R4 is a hydrogen atom, whereas the other radical is selected from the group consisting of “hydrogen atom, alkyl, aryl, heteroaryl, arylalkyl, heteroarylalkyl, heterocyclyl, heterocyclylalkyl, -alkyl-O-aryl, -alkyl-O-heteroaryl, -alkyl-O-heteroarylalkyl, -alkyl-O-heterocyclyl, alkyl-O-heterocyclylalkyl, -alkyl-CO-aryl, -alkyl-CO-arylalkyl, -alkyl-CO-heteroaryl, -alkyl-CO-heteroarylalkyl, -alkyl-CO-heterocyclyl, -alkyl-CO-heterocyclylalkyl, -alkyl-C(O)O-aryl, -alkyl-C(O)O-arylalkyl, -alkyl-C(O)O-heteroaryl, -alkyl-C(O)O-heteroarylalkyl, -alkyl-C(O)O-heterocyclyl, -alkyl-C(O)O-heterocyclylalkyl, -alkyl-CO—NH₂, -alkyl-CO—OH, -alkyl-NH₂, -alkyl-NH—C(NH)—NH2, alkylsulfonyl, arylsulfonyl, arylalkylsulfonyl, alkyl-S-alkyl, alkyl-S—H” which are optionally substituted in the aryl, heteroaryl, arylalkyl, heteroarylalkyl, heterocyclyl and/or heterocyclylalkyl group by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”; and preferably are selected from the group consisting of “arylalkyl, heteroarylalkyl, heterocyclylalkyl, -alkyl-O-aryl, -alkyl-O-arylalkyl, -alkyl-O-heteroaryl, -alkyl-O-heteroarylalkyl, -alkyl-O-heterocyclyl, alkyl-O-heterocyclylalkyl, -alkyl-CO-aryl, -alkyl-CO-arylalkyl, -alkyl-CO-heteroaryl, -alkyl-CO-heteroarylalkyl, -alkyl-CO-heterocyclyl, alkyl-CO-heterocyclylalkyl, -alkyl-C(O)O-aryl, -alkyl-C(O)O-arylalkyl, -alkyl-C(O)O-heteroaryl, -alkyl-C(O)O-heteroarylalkyl, -alkyl-C(O)O-heterocyclyl, -alkyl-C(0)0-heterocyclylalkyl, -alkyl-CO—NH₂, -alkyl-CO—OH, -alkyl-NH₂, -alkyl-NH—C(NH)—NH₂,” optionally being substituted in the aryl, heteroaryl, arylalkyl, heteroarylalkyl, heterocyclyl and/or heterocyclylalkyl group by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”;
R5 is selected from the group consisting of “hydrogen atom, alkyl, cycloalkyl, cycloalkylalkyl, aryl, heteroaryl, arylalkyl, heteroarylalkyl, heterocyclyl, heterocyclylalkyl, —CO-cycloalkyl, —CO-cycloalkylalkyl, —CO-aryl, —CO-arylalkyl, —CO-heteroaryl, —CO-heteroarylalkyl, —CO-heterocyclyl, —CO-heterocyclylalkyl, —CO—C*(R9R10)-NH2, —CO—CH2-C*(R9R10)-NH₂, —CO—C(R9R10)-CH2-NH2, alkylsulfonyl, arylsulfonyl, arylalkylsulfonyl” which are optionally substituted by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”; and preferably is selected from the group consisting of “hydrogen atom, —CO-alkyl, —CO-cycloalkyl, —CO-aryl, —CO-heteroaryl, —CO-arylalkyl, —CO-heteroarylalkyl, —CO-heterocyclyl, —CO—C*(R9R10)-NH₂, —CO—CH2-C*(R9R10)-NH₂, —CO—C*(R9R10)-CH2-NH₂, optionally being substituted by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”;
R6 is selected from the group consisting of “hydrogen atom, alkyl, cycloalkyl, cycloalkylalkyl” and preferably is a hydrogen atom;
R7 and R8 are independently of one another selected from the group consisting of “hydrogen atom, alkyl, cycloalkyl, cycloalkylalkyl” and preferably are a hydrogen atom;
R9 and R10 are independently of one another selected from the group consisting of “hydrogen atom, alkyl, natural alpha-amino acid side chain, unnatural alpha-amino acid side chain” and preferably are selected from the group consisting of “hydrogen atom, alkyl”;
m is 0, 1 or 2 and preferably is 0; and * means a carbon atom of R or S configuration when chiral;
   specific compounds of formula (D) include, but are not limited to:
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-phenethyl-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-phenethyl-4H-1,2,4-triazol-3-yl)-2-(1H-indol-3-yl)ethyl)-2-amino-2-methylpropanamide,
(R)—N-(1-(5-benzyl-4-(naphthalen-1-ylmethyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(naphthalen-1-ylmethyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(3-methoxybenzyl)-4H-1,2,4-triazol-3-yl)-2-(1H-indol-3-yl)ethyl)-2-amino-2-methylpropanamide,
(R)—N-(1-(4-(3-methoxybenzyl)-5-benzyl-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-benzyl-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-benzyl-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-(3-methoxybenzyl)-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-(naphthalen-1-ylmethyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(4-methoxybenzyl)-4H-1,2,4-triazol-3-yl)-2-(1H-indol-3-yl)ethyl)-2-amino-2-methylpropanamide,
(R)—N-(1-(4-(4-methoxybenzyl)-5-benzyl-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-(4-bromobenzyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-hexyl-4H-1,2,4-triazol-3-yl)-2-(1H-indol-3-yl)ethyl)-2-amino-2-methylpropanamide,
(R)—N-(1-(5-(3-(1H-indol-3-yl)propyl-4-hexyl-4H-1,2,4-triazol-3-yl)-2-(1H-indol-3-yl)ethyl)-2-amino-2-methylpropanamide,
(R)—N-(1-(4,5-bis(2-(1H-indol-3-yl)ethyl)-4H-1,2,4-triazol-3-yl)-2-(1H-indol-3-yl)ethyl)-2-amino-2-methylpropanamide,
(S)—N-(1-(5-(2-(1H-indol-3-yl)ethyl)-4-(2,4-dimethoxybenzyl)-4H-1,2,4-triazol-3-yl)-2-(1H-indol-3-yl)ethyl)-2-amino-2-methylpropanamide,
(R)—N-(1-(4-(3-methoxybenzyl)-5-phenethyl-4H-1,2,4-triazol-3-yl)-2-(1H-indol-3-yl)ethyl)-2-amino-2-methylpropanamide,
(R)—N-(1-(4-(4-methoxybenzyl)-5-phenethyl-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(3,5-dimethoxybenzyl)-4H-1,2,4-triazol-3-yl)-2-(1H-indol-3-yl)ethyl)-2-amino-2-methylpropanamide,
(R)—N-(1-(4-(4-methoxybenzyl)-5-(3-phenylpropyl)-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-(4-methoxybenzyl)-4H-1,2,4-triazol-3-yl)-2-(1H-indol-3-yl)ethyl)-2-amino-2-methylpropanamide,
(R)—N-(1-(4-(2-(1H-indol-3-yl)ethyl)-5-(3-(1H-indol-3-yl)propyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(2-methoxy)benzyl)-4H-1,2,4-triazol-3-yl)-2-(1H-indol-3-yl)ethyl)-2-amino-2-methylpropanamide,
(R)—N-(1-(4-(2-methoxybenzyl)-5-phenethyl-4H-1,2,4-triazol-3-yl)-2-(1H-indol-3-yl)ethyl)-2-amino-2-methylpropanamide,
(R)—N-(2-(1H-indol-3-yl)-1-(4-(naphthalen-1-ylmethyl)-5-phenethyl-4H-1,2,4-triazol-3-yl)ethyl)-2-amino-2-methylpropanamide,
(R)—N-(1-(5-(2-(1H-indol-3-yl)ethyl)-4-(3,4-dichlorobenzyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(4-fluorobenzyl)-4H-1,2,4 triazol-3-yl)-2-(1H-indol-3-yl)ethyl)-2-amino-2-methylpropanamide,
(R)—N-(1-(4-(4-fluorobenzyl)-5-benzyl-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)piperidine-4-carboxamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)piperidine-3-carboxamide,
(R)—N-(1-(4-(4-methylbenzyl)-5-(3-phenylpropyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(4-methylbenzyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)piperidine-2-carboxamide,
(R)—N-(1-(4-(4-methylbenzyl)-5-phenethyl-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)-2-aminobenzamide,
(R)—N-(1-(5-benzyl-4-(pyridin-2-ylmethyl)-4H-1,2,4-triazol-3-yl)-2-(1H-indol-3-yl)ethyl)-2-amino-2-methylpropanamide,
(2S,4R)—N—((R)-1-(4-(4-methoxybenzyl)-5-phenethyl-4H-1,2,4-triazol-3-yl)-2-(1H-indol-3-yl)ethyl)-4-hydroxypyrrolidine-2-carboxamide,
(S)—N—((R)-1-(4-(4-methoxybenzyl)-5-phenethyl-4H-1,2,4-triazol-3-yl)-2-(1H-indol-3-yl)ethyl)piperidine-3-carboxamide,
(R)—N—((R)-1-(4-(4-methoxybenzyl)-5-phenethyl-4H-1,2,4-triazol-3-yl)-2-(1H-indol-3-yl)ethyl)piperidine-3-carboxamide,
(R)—N-(1-(4-(4-ethylbenzyl)-5-phenethyl-4H-1,2,4-triazol-3-yl)-2-(1H-indol-3-yl)ethyl)-2-amino-2-methylpropanamide,
(R)—N-(1-(4-(4-methoxybenzyl)-5-phenethyl-4H-1,2,4-triazol-3-yl)-2-(1H-indol-3-yl)ethyl)piperidine-4-carboxamide,
(R)—N-(1-(5-(2-(1H-indol-3-yl)ethyl)-4-(4-methoxybenzyl)-4H-1,2,4-triazol-3-yl)-2-(1H-indol-3-yl)ethyl)piperidine-4-carboxamide,
(S)—N—((R)-1-(4-(4-methoxybenzyl)-5-phenethyl-4H-1,2,4-triazol-3-yl)-2-(1H-indol-3-yl)ethyl)pyrrolidine-2-carboxamide,
(R)—N—((R)-1-(4-(4-methoxybenzyl)-5-phenethyl-4H-1,2,4-triazol-3-yl)-2-(1H-indol-3-yl)ethyl)pyrrolidine-2-carboxamide,
(S)—N—((R)-1-(4-(4-methoxybenzyl)-5-phenethyl-4H-1,2,4-triazol-3-1)-2-(1H-indol-3-yl)ethyl)piperidine-2-carboxamide,
(R)—N—((R)-1-(4-(4-methoxybenzyl)-5-phenethyl-4H-1,2,4-triazol-3-yl)-2-(1H-indol-3-yl)ethyl)piperidine-2-carboxamide,
(R)—N-(1-(4-(4-methoxybenzyl)-5-phenethyl-4H-1,2,4-triazol-3-yl)-2-(1H-indol-3-yl)ethyl)-2-aminoacetamide,
(R)—N-(1-(4-(4-methoxybenzyl)-5-phenethyl-4H-1,2,4-triazol-3-yl)-2-(1H-indol-3-yl)ethyl)-2-(pyridin-2-yl)acetamide,
(R)—N-(1-(4-(4-methoxybenzyl)-5-phenethyl-4H-1,2,4-triazol-3-yl)-2-(1H-indol-3-yl)ethyl)-2-(pyridin-4-yl)acetamide,
(R)—N-(1-(4-(4-methoxybenzyl)-5-phenethyl-4H-1,2,4-triazol-3-yl)-2-(1H-indol-3-yl)ethyl)cyclohexanecarboxamide,
(R)—N-(1-(5-(2-(1H-indol-3-yl)ethyl)-4-benzyl-4H-1,2,4-triazol-3-yl)-2-(1H-indol-3-yl)ethyl)piperidine-4-carboxamide,
(R)—N-(1-(5-(2-(1H-indol-3-yl)ethyl)-4-benzyl-4H-1,2,4-trioazol-3-yl)-2-(1H-indol-3-yl)ethyl)-3-aminopropanamide,
(R)—N-(1-(4-(4-methoxybenzyl)-5-phenethyl-4H-1,2,4-triazol-3-yl)-2-(1H-indol-3-yl)ethyl)-3-aminopropanamide,
(S)—N—((R)-1-(4-(4-methoxybenzyl)-5-phenethyl-4H-1,2,4-triazol-3-yl)-2-(1H-indol-3-yl)ethyl)-2-aminopropanamide,
(R)—N-(1-(4-(4-methoxybenzyl)-5-phenethyl-4H-1,2,4-triazol-3-yl)-2-(1H-indol-3-yl)ethyl)-2-(pyridin-3-yl)acetamide,
(R)—N-(1-(4-(4-methoxybenzyl)-5-phenethyl-4H-1,2,4-triazol-3-yl)-2-(1H-indol-3-yl)ethyl)-3-(pyridin-3-yl)propanamide,
(R)—N-(1-(5-(2-(1H-indol-3-yl)ethyl)-4-benzyl-4H-1,2,4-triazol-3-yl)-2-(1H-indol-3-yl)ethyl)-2-(pyridin-2-yl)acetamide,
(R)—N-(1-(5-(2-(1H-indol-3-yl)ethyl)-4-(2,4-dimethoxybenzyl)-4H-1,2,4-triazol-3-yl)-2-(1H-1-indol-3-yl)ethyl)-2-(pyridin-2-yl)acetamide,
(R)—N-(1-(4-(2,4-dimethoxybenzyl)-5-phenethyl-4H-1,2,4-triazol-3-yl)-2-(1H-indol-3-yl)ethyl)piperidine-4-carboxamide,
(R)—N-1-(4-(2,4-dimethoxybenzyl)-5-phenethyl-4H-1,2,4-triazol-3-yl)-2-(1H-indol-3-yl)ethyl)piperidine-2-carboxamide,
(R)—N-(1-(4-(2,4-dimethoxybenzyl)-5-phenethyl-4H-1,2,4-triazol-3-yl)-2-(1H-indol-3-yl)ethyl)picolinamide,
(R)—N-(1-(4-(2,4-dimethoxybenzyl)-5-phenethyl-4H-1,2,4-triazol-3-yl)-2-(1H-indol-3-yl)ethyl)isonicotinamide,
(R)—N-(1-(4-(2,4-dimethoxybenzyl)-5-phenethyl-4H-1,2,4-triazol-3-yl)-2-(1H-indol-3-yl)ethyl)pyrazine-2-carboxamide,
(R)—N-1-(4-(2,4-dimethoxybenzyl)-5-phenethyl-4H-1,2,4-triazol-3-yl)-2-(1H-indol-3-yl)ethyl)piperazine-2-carboxamide,
(R)—N-1-(4-(2,4-dimethoxybenzyl)-5-phenethyl-4H-1,2,4-triazol-3-yl)-2-(1H-indol-3-yl)ethyl)pyrrolidine-2-carb oxamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)-2-aminoacetamide,
(S)—N—((R)-1-(5-(2-(1 W-indol-3-yl)ethyl)-4-(2,4-dimethoxybenzyl)-4H-1,2,4-triazol-3-yl)-2-(1H-indol-3-yl)ethyl)pyrrolidine-2-carb oxamide
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)pyrazine-2-carboxamide,
(R)—N-1-(5-(2-(1H-indol-3-yl)ethyl)-4-(2,4-dimethoxybenzyl)-4H-1,2,4-triazol-3-yl)-2-(1H-indol-3-yl)ethyl)piperazine-carboxamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)picolinamide,
(R)-1-(5-(2-(1H-indol-3-yl)ethyl)-4-(2,4-dimethoxybenzyl)-4H-1,2,4-triazol-3-yl)-2-(1H-indol-3-yl)ethanamine,
(R)—N-(1-(4-(2,4-dimethoxybenzyl)-5-phenethyl-4H-1,2,4-triazol-3-yl)-2-(1H-indol-3-yl)ethyl)-2-aminoacetamide,
(R)—N-(1-(4-(4-methoxybenzyl)-5-phenethyl-4H-1,2,4-triazol-3-yl)-2-(1H-indol-3-yl)ethyl)pyrazine-2-carb oxamide,
(R)—N-(1-(4-(4-methoxybenzyl)-5-phenethyl-4H-1,2,4-triazol-3-yl)-2-(1H-indol-3-yl)ethyl)isonicotinamide,
(R)—N-1-(4-(4-methoxybenzyl)-5-phenethyl-4H-1,2,4-triazol-3-yl)-2-(1H-indol-3-yl)ethyl)piperazine-2-carboxamide,
(R)—N-(1-(4-(4-methoxybenzyl)-5-phenethyl-4H-1,2,4-triazol-3-yl)-2-(1H-indol-3-yl)ethyl)picolinamide,
(R)—N-(1-(5-(2-(1H-indol-3-yl)ethyl)-4-(4-methoxybenzyl)-4H-1,2,4-triazol-3-yl)-2-(1H-indol-3-yl)ethyl)picolinamide,
(R)—N-(1-(5-(2-(1H-indol-3-yl)ethyl)-4-(4-methoxybenzyl)-4H-1,2,4-triazol-3-yl)-2-(1H-indol-3-yl)ethyl)piperazine-2-carboxamide,
(R)—N-(1-(4-(4-ethylbenzyl)-5-phenethyl-4H-1,2,4-triazol-3-yl)-2-(1H-indol-3-yl)ethyl)-2-(pyridin-2-yl)acetamide,
(R)—N-(1-(4-(4-ethylbenzyl)-5-phenethyl-4H-1,2,4-triazol-3-yl)-2-(1H-indol-3-yl)ethyl)piperidine-4-carboxamide,
(R)—N-1-(4-(4-ethylbenzyl)-5-phenethyl-4H-1,2,4-triazol-3-yl)-2-(1H-indol-3-yl)ethyl)piperazine-2-carboxamide,
(R)—N-(1-(4-(4-ethylbenzyl)-5-phenethyl-4H-1,2,4-triazol-3-yl)-2-(1H-indol-3-yl)ethyl)pyrazine-2-carboxamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)-2-cis-aminocyclohexanecarboxamide,
(S)—N—((R)-1-(5-(2-(1H-indol-3-yl)ethyl)-4-(4-methoxybenzyl)-4H-1,2,4-triazol-3-yl)-2-(1H-indol-3-yl)ethyl)piperidine-3-carboxamide)
(R)—N—((R)-1-(5-(2-(1H-indol-3-yl)ethyl)-4-(4-methoxybenzyl)-4H-1,2,4-triazol-3-yl)-2-(1H-indol-3-yl)ethyl)piperidine-2-carboxamicle,
(S)—N—((R)-1-(5-(2-(1H-indol-3-yl)ethyl)-4-(4-methoxybenzyl)-4H-1,2,4-triazol-3-yl)-2-(1H-indol-3-yl)ethyl)pyrrolidine-2-carboxamide,
(R)—N—((R)-1-(5-(2-(1H-indol-3-yl)ethyl)-4-(4-methoxybenzyl)-4H-1,2,4-triazol-3-yl)-2-(1H-indol-3-yl)ethyl)pyrrolidine-2-carboxamide,
(R)—N-(1-(5-(2-(1H-indol-3-yl)ethyl)-4-(4-methoxybenzyl)-4H-1,2,4-triazol-3-yl)-2-(1H-indol-3-yl)ethyl)-2-(pyridin-2-yl)acetamide,
(R)—N-(1-(5-(2-(1H-indol-3-yl)ethyl)-4-(4-bromobenzyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(4-methoxybenzyl)-4H-1,2,4-triazol-3-yl)-2-phenylethyl)-2-amino-2-methylpropanamide,
(R)—N-(2-(1H-indol-3-yl)-1-(5-phenethyl-4-(thiophen-2-ylmethyl)-4H-1,2,4-triazol-3-yl)ethyl)piperidine-4-carboxamide,
(R)—N-(1-(4-(2-(1H-indol-3-yl)ethyl)-4H-1,2,4-triazol-3-yl)-2-(1H-indol-3-yl)ethyl)-2-amino-2-methylpropanamide,
(R)—N-(1-(5-((1H-indol-3-yl)methyl)-4-methyl-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-methyl-4H-1,2,4-triazol-3-yl)-2-(1H-indol-3-yl)ethyl)-2-amino-2-methylpropanamide,
(R)—N-(1-(5-((1H-indol-3-yl)methyl)-4H-1,2,4-triazol-3-yl)-2-(1H-indol-3-yl)ethyl)-2-amino-2-methylpropanamide,
(R)—N-(1-(5-((1AV-indol-3-yl)methyl)-4-(2,4-dimethoxybenzyl)-4H-1,2,4-triazol-3-yl)-2-(1H-indol-3-yl)ethyl)-2-amino-2-methylpropanamide,
(R)—N-(1-(4-(2,4-dimethoxybenzyl)-5-methyl-4H-1,2,4-triazol-3-yl)-2-(1H-indol-3-yl)ethyl)-2-amino-2-methylpropanamide,
(R)—N-(1-(5-((1H-indol-3-yl)methyl)-4-(4-methoxybenzyl)-4H-1,2,4-triazol-3-yl)-2-(1H-indol-3-yl)ethyl)-2-amino-2-methylpropanamide,
(R)—N-(1-(4-(2,4-dimethoxybenzyl)-5-benzyl-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-(2,4-dimethoxybenzyl)-4H-1,2,4-triazol-3-yl)-2-(1H-indol-3-yl)ethyl)-2-amino-2-methylpropanamide,
(R)—N-(1-(5-((1H-indol-3-yl)methyl)-4-phenethyl-4H-1,2,4-triazol-3-yl)-2-(1H-indol-3-yl)ethyl)-2-amino-2-methylpropanamide,
(R)—N-(1-(5-benzyl-4-phenethyl-4H-1,2,4-triazol-3-yl)-2-(1H-indol-3-yl)ethyl)-2-amino-2-methylpropanamide,
(R)—N-(1-(5-benzyl-4-(2,2-diphenylethyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(2,2-diphenylethyl)-4H-1,2,4-triazol-3-yl)-2-(1H-indol-3-yl)ethyl)-2-amino-2-methylpropanamide,
(R)—N-(1-(4-(3,5-dimethoxybenzyl)-5-benzyl-4H-1,2,4-triazol-3-yl)-2-(1H-indol-3-yl)ethyl)-2-amino-2-methylpropanamide,
(R)—N-(1-(4,5-dibenzyl-4H-1,2,4-triazol-3-yl)-2-(1H-indol-3-yl)ethyl)-2-amino-2-methylpropanamide,
(R)—N-(1-(5-benzyl-4-hexyl-4H-1,2,4-triazol-3-yl)-2-(1H-indol-3-yl)ethyl)-2-amino-2-methylpropanamide,
(R)—N-(1-(4-(2-(1H-indol-3-yl)ethyl)-5-benzyl-4H-1,2,4-triazol-3-yl)-2-(1H-indol-3-yl)ethyl)-2-amino-2-methylpropanamide,
(S)—N-(1-(4-(2,4-dimethoxybenzyl)-5-benzyl-4H-1,2,4-triazol-3-yl)-2-(1H-indol-3-yl)ethyl)-2-amino-2-methylpropanamide,
(R)—N-(1-(4-(3,5-dimethoxybenzyl)-5-phenethyl-4H-1,2,4-triazol-3-yl)-2-(1H-indol-3-yl)ethyl)-2-amino-2-methylpropanamide,
(R)—N-(1-(4-(4-bromobenzyl)-5-benzyl-4H-1,2,4-triazol-3-yl)-2-(1H-indol-3-yl)ethyl)-2-amino-2-methylpropanamide,
(R)—N-(1-(4-(2-methoxybenzyl)-5-benzyl-4H-1,2,4-triazol-3-yl)-2-(1H-indol-3-yl)ethyl)-2-amino-2-methylpropanamide,
(S)—N-(1-(4-(2,4-dimethoxybenzyl)-5-phenethyl-4H-1,2,4-triazol-3-yl)-2-(1H-indol-3-yl)ethyl)-2-amino-2-methylpropanamide,
(R)—N-(1-(4,5-diphenethyl-4H-1,2,4-triazol-3-yl)-2-(1H-indol-3-yl)ethyl)-2-amino-2
(R)—N-(1-(4-(3,4-dichlorobenzyl)-5-benzyl-4H-1,2,4-triazol-3-yl)-2-(1H-indol-3-yl)ethyl)-2-amino-2-methylpropanamide,
(R)-1-(5-(2-(1H-indol-3-yl)ethyl)-4-(2,4-dimethoxybenzyl)-4H-1,2,4-triazol-3-yl)-2-(1H-indol-3-yl)ethanamine,
(R)—N-(1-(4-(4-methoxybenzyl)-5-benzyl-4H-1,2,4-triazol-3-yl)-2-phenylethyl)-2-amino-2-methylpropanamide,
(R)—N-(1-(4-(4-fluorobenzyl)-5-phenethyl-4H-1,2,4-triazol-3-yl)-2-(1H-indol-3-yl)ethyl)-2-amino-2-methylpropanamide,
(R)—N-(1-(4-(3,4-dichlorobenzyl)-5-phenethyl-4H-1,2,4-triazol-3-yl)-2-(1H-indol-3-yl)ethyl)-2-amino-2-methylpropanamide,
(R)—N-(1-(4-(4-methylbenzyl)-5-benzyl-4H-1,2,4-triazol-3-yl)-2-(1H-indol-3-yl)ethyl)-2-amino-2-methylpropanamide,
(S)—N-(1-(4-(4-methoxybenzyl)-5-(3-phenylpropyl)-4H-1,2,4-triazol-3-yl)-2-(1H-indol-3-yl)ethyl)-2-amino-2-methylpropanamide,
(S)—N-(1-(4-(4-methoxybenzyl)-5-benzyl-4H-1,2,4-triazol-3-yl)-2-(1H-indol-3-yl)ethyl)-2-amino-2-methylpropanamide,
N—((R)-1-(4-(4-nitrobenzyl)-5-phenethyl-4H-1,2,4-triazol-3-yl)-2-(1H-indol-3-yl)ethyl)-2-amino-2-methylpropanamide,
(S)—N-(1-(4-(4-methoxybenzyl)-5-phenethyl-4H-1,2,4-triazol-3-yl)-2-(1H-indol-3-yl)ethyl)-2-amino-2-methylpropanamide,
(R)—N-(1-(4-(4-methoxyphenethyl)-5-phenethyl-4H-1,2,4-triazol-3-yl)-2-(1H-indol-3-yl)ethyl)-2-amino-2-methylpropanamide,
(R)—N-(2-(1H-indol-3-yl)-1-(5-phenethyl-4-(thiophen-2-ylmethyl)-4H-1,2,4-triazol-3-yl)ethyl)-2-amino-2-methylpropanamide,
(R)—N-(2-(1H-indol-3-yl)-1-(5-phenethyl-4-(pyridin-2-ylmethyl)-4H-1,2,4-triazol-3-yl)ethyl)-2-amino-2-methylpropanamide,
(R)—N-(2-(1H-indol-3-yl)-1-(5-phenethyl-4-(pyridin-2-ylmethyl)-4H-1,2,4-triazol-3-yl)ethyl)piperidine-3-carboxamide,
(S)—N-1-(4-(4-ethylbenzyl)-5-phenethyl-4H-1,2,4-triazol-3-yl)-2-(1H-indol-3-yl)ethyl)pyrrolidine-2-carboxamide,
N—((R)-1-(4-(4-ethylbenzyl)-5-phenethyl-4H-1,2,4-triazol-3-yl)-2-(1H-indol-3-yl)ethyl)-2-aminoacetamide,
N—((R)-1-(5-(2-(1H-indol-3-yl)ethyl)-4-(4-methoxybenzyl)-4H-1,2,4-triazol-3-yl)-2-(1H-indol-3-yl)ethyl)-2-(pyridin-4-yl)acetamide,
(2R)—N—((R)-1-(4-(4-ethylbenzyl)-5-phenethyl-4H-1,2,4-triazol-3-yl)-2-(1H-indol-3-N—((R)-1-(4-(4-ethylbenzyl)-5-phenethyl-4H-1,2,4-triazol-3-yl)-2-(1H-indol-3-yl)ethyl)picolinamide,
N—((R)-1-(4-(4-ethylbenzyl)-5-phenethyl-4H-1,2,4-triazol-3-yl)-2-(1H-indol-3-yl)ethyl)-2-aminopyridine-3-carboxamide,
(2S)—N—((R)-1-(4-(4-ethylbenzyl)-5-phenethyl-4H-1,2,4-triazol-3-yl)-2-(1H-indol-3-yl)ethyl)-2-aminopropanamide,
N—((R)-1-(4-(4-ethylbenzyl)-5-phenethyl-4H-1,2,4-triazol-3-yl)-2-(1H-indol-3-yl)ethyl)isonicotinamide,
N—((R)-2-(1H-indol-3-yl)-1-(5-phenethyl-4-phenyl-4H-1,2,4-triazol-3-yl)ethyl)piperidine-4-carboxamide,
(2S)—N—((R)-2-(1H-indol-3-yl)-1-(5-phenethyl-4-phenyl-4H-1,2,4-triazol-3-yl)ethyl)pyrrolidine-2-carboxamide,
N—((R)-2-(1H-indol-3-yl)-1-(5-phenethyl-4-phenyl-4H-1,2,4-triazol-3-yl)ethyl)-2-aminoacetamide,
N—((R)-2-(1H-indol-3-yl)-1-(5-phenethyl-4-phenyl-4H-1,2,4-triazol-3-yl)ethyl)-2-(pyridin-2-yl)acetamide,
N—((R)-2-(1H-indol-3-yl)-1-(5-phenethyl-4-phenyl-4H-1,2,4-triazol-3-yl)ethyl)picolinamide,
N—((R)-1-(5-(2-(1H-indol-3-yl)ethyl)-4-(4-ethylphenyl)-4H-1,2,4-triazol-3-yl)-2-(1H-indol-3-yl)ethyl)picolinamide,
N—((R)-1-(5-(2-(1H-indol-3-yl)ethyl)-4-(4-ethylphenyl)-4H-1,2,4-triazol-3-yl)-2-(1H-indol-3-yl)ethyl)-2-(pyridin-2-yl)acetamide,
N—((R)-1-(5-(2-(1H-indol-3-yl)ethyl)-4-(4-ethylphenyl)-4H-1,2,4-triazol-3-yl)-2-(1H-indol-3-yl)ethyl)-2-aminoacetamide,
(2S)—N—((R)-1-(5-(2-(1H-indol-3-yl)ethyl)-4-(4-ethylphenyl)-4H-1,2,4-triazol-3-yl)-2-(1H-indol-3-yl)ethyl)pyrrolidine-2-carboxamide,
N—((R)-1-(5-(2-(1H-indol-3-yl)ethyl)-4-(4-methoxybenzyl)-4H-1,2,4-triazol-3-yl)-2-(1H-indol-3-yl)ethyl)-2-aminoacetamide,
N—((R)-1-(4-(4-methoxybenzyl)-5-phenethyl-4H-1,2,4-triazol-3-yl)-2-(1H-indol-3-yl)ethyl)-2-trans-aminocyclohexanecarboxamide,
N—((R)-1-(4-(4-ethylbenzyl)-5-phenethyl-4H-1,2,4-triazol-3-yl)-2-(1H-indol-3-yl)ethyl)-2-(pyridin-3-yl)acetamide,
(3S)—N—((R)-1-(4-(4-ethylbenzyl)-5-phenethyl-4H-1,2,4-triazol-3-yl)-2-(1H-indol-3-yl)ethyl)piperidine-3-carboxamide,
N—((R)-1-(4-(4-ethylbenzyl)-5-phenethyl-4H-1,2,4-triazol-3-yl)-2-(1H-indol-3-yl)ethyl)-2-aminobenzamide,
N—((R)-1-(5-(2-(1H-indol-3-yl)ethyl)-4-phenyl-4H-1,2,4-triazol-3-yl)-2-(1H-indol-3-yl)ethyl)picolinamide,
N—((R)-1-(5-(2-(1H-indol-3-yl)ethyl)-4-phenyl-4H-1,2,4-triazol-3-yl)-2-(1H-indol-3-yl)ethyl)piperidine-4-carboxamide,
N—((R)-2-(1H-indol-3-yl)-1-(4-(2,4-dimethoxyphenyl)-5-phenethyl-4H-1,2,4-triazol-3-yl)ethyl)picolinamide,
N—((R)-2-(1H-indol-3-yl)-1-(4-(2,4-dimethoxyphenyl)-5-phenethyl-4H-1,2,4-triazol-3-yl)ethyl)-2-(pyridin-2-yl)acetamide,
N—((R)-2-(1H-indol-3-yl)-1-(4-(2,4-dimethoxyphenyl)-5-phenethyl-H-1,2,4-triazol-3-yl)ethyl)pyrazine-2-carboxamide,
N—((R)-2-(1H-indol-3-yl)-1-(4-(2,4-dimethoxyphenyl)-5-phenethyl-4H-1,2,4-triazol-3-yl)ethyl)-2-aminoacetamide,
N—((R)-2-(1H-indol-3-yl)-1-(4-(2,4-dimethoxyphenyl)-5-phenethyl-4H-2,4-triazol-3-yl)ethyl)piperidine-4-carboxamide,
N—((R)-1-(5-benzyl-4-((pyridin-2-yl)methyl)-4H-1,2,4-triazol-3-yl)-2-(1H-indol-3-yl)ethyl)picolinamide,
N—((R)-1-(5-benzyl-4-((pyridin-2-yl)methyl)-4H-1,2,4-triazol-3-yl)-2-1H-indol-3-yl)ethyl)-2-amino-acetamide,
N—((R)-1-(5-benzyl-4-((pyridin-2-yl)methyl)-4H-1,2,4-triazol-3-yl)-2-(1H-indol-3-yl)ethyl)piperidine-4-carboxamide,
N—((R)-1-(5-benzyl-4-((pyridin-4-yl)methyl)-4H-1,2,4-triazol-3-yl)-2-(1H-indol-3-yl)ethyl)-2-amino-2-methylpropanamide,
N—((R)-1-(5-(4-methoxybenzyl)-4-phenethyl-4H-1,2,4-triazol-3-yl)-2-(1H-indol-3-yl)ethyl)-2-amino-2-methylpropanamide,
N—((R)-1-(5-benzyl-4-((pyridin-4-yl)methyl)-4H-1,2,4-triazol-3-yl)-2-(1H-indol-3-yl)ethyl)picolinamide,
N—((R)-1-(5-benzyl-4-((pyridin-4-yl)methyl)-4H-1,2,4-triazol-3-yl)-2-(1H-indol-3-yl)ethyl)-2-amino-acetamide,
(R)-benzyl-3-(2-aminoisobutyramido)-3-(5-(2-(1H-indol-3-yl)ethyl)-4-(4-methoxybenzyl)-4H-1,2,4-triazol-3-yl)-propanoate,
N—((R)-1-(5-benzyl-4-((pyridin-3-yl)methyl)-4H-1,2,4-triazol-3-yl)-2-(1H-indol-3-yl)ethyl)-2-amino-2-methylpropanamide,
N—((R)-1-(4-benzyl-5-phenethyl-4H-1,2,4-triazol-3-yl)-2-(1H-indol-3-yl)ethyl)-2-amino-2-methylpropanamide,
N—((R)-2-(1H-indol-3-yl)-1-(4-methyl-5-phenethyl-4H-1,2,4-triazol-3-yl)ethyl)picolinamide,
N—((R)-1-(5-(2-(1H-indol-3-yl)ethyl)-4-phenyl-4H-1,2,4-triazol-3-yl)-2-(1H-indol-3-yl)ethyl)-2-amino-2-methylpropanamide,
N—((R)-1-(4-(4-methoxybenzyl)-5-phenethyl-4H-1,2,4-triazol-3-yl)-2-(1H-indol-3-yl)ethyl)benzamide,
(R)-1-(4-(2,4-dimethoxybenzyl)-5-phenethyl-4H-1,2,4-triazol-3-yl)-2-(1H-indol-3-yl)-N-phenylmethanesulfonylamine,
(R)-1-(4-(2,4-dimethoxyben2yl)-5-phenethyl-4H-1,2,4-triazol-3-yl)-2-(1H-indol-3-yl)-N-tosylethanamine,
N—((R)-1-(4-(2,4-dimethoxybenzyl)-5-phenethyl-4H-1,2,4-triazol-3-yl)-2-(1H-indol-3-yl)ethyl)-2-amino-2-methylpropanamide,
N-1-((R)-1-(4-(2,4-dimethoxybenzyl)-5-phenethyl-4H-1,2,4-triazol-3-yl)-2-(1H-indol-3-yl)ethyl)ethane-1,2-diamine,
N—((R)-1-(4-((furan-2-yl)methyl)-5-phenethyl-4H-1,2,4-triazol-3-yl)-2-(1H-indol-3-yl)ethyl)-2-amino-2-methylpropanamide,
N—((R)-1-(4-((furan-2-yl)methyl)-5-phenethyl-4H-1,2,4-triazol-3-yl)-2-(1H-indol-3-yl)ethyl)picolinamide,
N—((R)-1-(4-((furan-2-yl)methyl)-5-phenethyl-4H-1,2,4-triazol-3-yl)-2-(1H-indol-3-yl)ethyl)piperidine-4-carboxamide,
N—((R)-1-(4-(4-methoxybenzyl)-5-phenethyl-4H-1,2,4-triazol-3-yl)-2-(1H-indol-3-yl)ethyl)-tetrahydro-2W-pyran-4-carboxamide
N—((R)-1-(5-((1H-indol-3-yl)methyl)-4-(3-methoxybenzyl)-4H-1,2,4-triazol-3-yl)-2-(1H-indol-3-yl)ethyl)-2-amino-2-methylpropanamide,
(2S)—N—((R)-1-(4-(4-methoxybenzyl)-5-phenethyl-4H-1,2,4-triazol-3-yl)-2-(1H-indol-3-yl)ethyl)-2-amino-3-phenylpropanamide,
(R)-1-(5-(2-(1H-indol-3-yl)ethyl)-4-(2,4-dimethoxybenzyl)-4H-1,2,4-triazol-3-yl)-2-(1H-indol-3-yl)-N-tosylethanamine,
N—((R)-1-(4-(2,4-dimethoxybenzyl)-5-phenethyl-4H-1,2,4-triazol-3-yl)-2-(1H-indol-3-yl)ethyl)-4-azidobenzamide,
N-benzyl-(R)-1-(4-(2,4-dimethoxybenzyl)-5-phenethyl-4H-1,2,4-triazol-3-yl)-2-(1H-indol-3-yl)ethanamine,
(2S)—N—((R)-1-(5-(2-(1H-indol-3-yl)ethyl)-4-(4-methoxybenzyl)-4H-1,2,4-triazol-3-yl)-2-(1H-indol-3-yl)ethyl)-2,5-dihydro-1H-pyrrole-2-carboxamide;
   peptides having the formula (E)

Gly-Ser-Ser(Octanoyl)-Phe A (E)
where A is —OH, —NH₂, -Leu-Ser-Pro-Glu-B, or -Ala-Lys-Leu-Gln-Pro-Arg-B,
where B is —OH or —NH₂;
   peptides selected from the group, consisting of,
rPKPfQwFwLL-NH₂
PKPfQwFwLL-NH₂
KPfQwFwLL-NH₂
PfQwFWLL-NH₂
fQwFwLL-NH₂
rPKPAQwFwLL-NH₂
rPKPfAwFwLL-NH₂
rPKPfQwAwLL-NH₂
rPKPfQwFwLA-NH₂
rPKPfQWFwLL-NH₂
rPKPFQwFwLL-NH₂
rPKPyQwFwLL-NH₂
rPKPwQwFwLL-NH₂
rPKP QwFwLL-NH₂;
wherein r is D-arginine, P is proline, K is lysine, F is phenylalanine, f is D-phenylalanine, Q is glutamine, w is D-tryptophan, L is leucine, A is alanine, y is D-tyrosine.
   compounds having the formula (F):

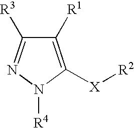

wherein X is selected from the group consisting of:
(1) bond,
(2) —(CH₂)ₘ—,
(3) —(CH₂)ₘC₂-₆heterocycloalkyl-,
(4) —(CH₂)ₙC₂-₆heterocycloalkyl-(CH₂)ₙ—NR⁶—,
(5) —NR⁶—(CH₂)ₙC₃-₆cycloalkyl-(CH₂)ₙ—NR⁶—,
(7) —NR⁶—(CH₂)ₘ—,
(8) —(CH₂)ₙ—NR₆—(CH₂)ₘ—NR⁶—,
(9) —NR⁶—C₂-₆alkenyl-,
(10) —NR⁶—C₂-₆alkynyl-,
(11) —NR⁶-phenyl-,
(12) —NR⁶-phenyl-NR⁶—,
(13) —NR⁶—(CH₂)ₙ—C₂-₆heterocycloalkyl-,
(14) —NR⁶—(CH₂)n-heteroaryl-, and
(15) —NR⁶-heteroaryl-NR⁶—
wherein alkenyl, alkynyl, cycloalkyl, heterocycloalkyl, phenyl, heteroaryl, and (CH₂) are unsubstituted or substituted with 1-4 substituents selected from oxo, halogen and C₁-₄alkyl;
R¹ is selected from the group consisting of
(1) hydrogen,
(2) —CF₃,
(3) halogen,
(4) —C₁-₈alkyl,
(5) —C₂-₈alkenyl,
(6) —C₂-₈alkynyl,
(7) —(CH₂)ₙOH,
(8) —(CH₂)ₙphenyl,
(9) —(CH₂)ₙheteroaryl,
(10) —(CH₂)ₙC₃-₇cycloalkyl,
(11) —(CH2)ₙC₂-₉heterocycloalkyl,
(12) —(CH₂)ₙN(R⁶)CH₂phenyl,
(13) —(CH₂)ₙN(R⁶)C(O)phenyl,
(14) —(CH2)ₙN(R⁶)C(O)heteroaryl,
(15) —CN,
(16) —C(O)R⁵,
(17) —C(O)C₂-₈alkenyl,
(18) —C(O)C₂₋₈alkynyl,
(19) —C(O)C₃-₇cycloalkyl,
(20) —C(O)C₂-₉heterocycloalkyl,
(21) —CO₂R⁵,
(22) —C(O)N(R⁶)₂, and
(23) —(CH₂)₃-₇R²,
wherein alkyl, alkenyl, alkynyl, phenyl, heteroaryl, heterocycloalkyl, and cycloalkyl are unsubstituted or substituted with one to three groups independently selected from CF₃, C₁-₄ alkoxy, C₁-₄ alkyl, halogen and phenyl, wherein the phenyl substituent is unsubstituted or substituted with CF₃, C₁-₄ alkoxy, C₁-₄ alkyl and halogen;
R² is selected from the group consisting of
(1) hydrogen,
(2) —C₁-₈alkyl,
(3) —C₂-₈alkenyl,
(4) —C₂-₈alkynyl,
(5) —(CH₂)ₙC₃-₇cycloalkyl,
(6) —(CH₂)ₙC₂-₉heterocycloalkyl,
(7) —(CH₂)ₙphenyl,
(8) —(CH₂)ₙnaphthyl,
(9) —(CH₂)ₙheteroaryl,
(10) —OR⁶,
(11) —C(O)R⁶,
(12) ═CH—N(R⁶)₂,
(13) —(CH₂)ₙN(R⁶)₂,
(14) —(CH₂)ₙN(R⁶)CO₂C₁-₈alkyl,
(15) —(CH₂)ₙCO₂H,
(16) —C(O)C₁-₈alkyl,
(17) —C(O)C₃-₇cycloalky,
(18) —C(O)C₂-₉heterocycloalkyl,
(19) —C(O)(CH₂)ₙaryl,
(20) —C(O)(CH₂)ₙheteroaryl,
(21) —C(O)CF₃,
(22) —C(O)(CH₂)ₙN(R⁶)₂,
(23) —C(O)N(R⁶)C₁-₈alkyl,
(24) —C(O)N(R⁶)(CH₂)ₙC₃-₇cycloalkyl,
(25) —C(O)N(R⁶)(CH₂)ₙC₂-₇heterocycloalkyl,
(26) —C(O)N(R⁶)(CH₂)ₙphenyl,
(27) —C(O)N(R⁶)(CH₂)ₙnaphthyl,
(28) —C(O)N(R⁶)(CH²)ₙheteroaryl,
(29) —C(S)N(R⁶)(CH₂)ₙphenyl,
(30) —CO₂C₁-₈alkyl,
(31) —CO₂(CH₂)ₙC₃-₇cycloalkyl
(32) —CO₂(CH₂)ₙC₂-₉heterocycloalkyl
(33) —CO₂(CH₂)ₙphenyl,
(34) —CO₂(CH₂)ₙnaphthyl,
(35) —CO₂(CH₂)ₙheteroaryl,
(36) —SO₂C₁-₈alkyl,
(37) —SO₂C₃-₇cycloalkyl,
(38) —SO₂C₂-₉heterocycloalkyl,
(39) —SO₂phenyl,
(40) —SO₂naphthyl,
(41) —SO₂heteroaryl,
(42) —S(O)N(R⁶)phenyl,
(43) —S—C₁-₈alkyl,
(44) —S—C₃-₇cycloalkyl,
(45) —S—C₂-₉heterocycloalkyl.
(46) —S-phenyl,
(47) —S-naphthyl, and
(48) —S-heteroaryl,
wherein alkyl, alkenyl, alkynyl, cycloalkyl, heterocycloalkyl, aryl, phenyl, naphthyl, heteroaryl, and (CH₂) are unsubstituted or substituted with one to four substituents independently selected from R⁷, and wherein two C₁-₄ alkyl substituents on the same (CH₂) carbon may cyclize to form a 3- to 6-membered ring, provided that when X is a bond or —(CH₂)ₘ, then R² is not hydrogen, —C₁-₈alkyl, —C₂-₈alkenyl, —C₂-₈alkynyl, —(CH₂)ₙC₃-₇cycloalkyl, —C₂-₉heterocycloalkyl, -phenyl, -benzyl, -naphthyl, -heteroaryl, —OR⁶, —C(O)R⁶, or —S—C₁-₈alkyl, further provided that when X is a bond R² is not —NH₂, —CO₂C₁-₈alkyl, —CO₂C₃-₇cycloalkyl, —CO₂(CH₂)₀-₁phenyl, and provided that when X is —(CH₂)ₘNR⁶— then R² is not —C(O)R⁶;
R³ is selected from the group consisting of:
(1) —C₁-₈alkyl,
(2) —(CH₂)ₙ-phenyl)
(3) —(CH₂)ₙ-naphthyl,
(4) —(CH₂)ₙC₃-₇cycloalkyl,
(5) —C(O)C₁-₈alkyl,
(6) —CO₂R⁵,
(7) —C(O)N(R⁶)OC₁-₈alkyl,
(8) —C(O)C₁-₄alkenylphenyl,
(9) —C(O)C₁-₄alkynylphenyl,
(10) —C(O)phenyl,
(11) —C(O)naphthyl,
(12) —C(O)heteroaryl, and
(13) —C(O)C₃-₇cycloalkyl,
wherein alkyl, alkenyl, alkynyl, phenyl, naphthyl, heteroaryl, and cycloalkyl are unsubstituted or substituted with one to three groups independently selected from R⁸, and each (CH₂)ₙ is unsubstituted or substituted with 1 to 2 groups independently selected from:
C₁-₄alkyl, —OH, halogen, and C₁-₄ alkenyl;
R⁴ is selected from the group consisting of:
(1) —(CH₂)n-phenyl,
(2) —(CH₂)n-naphthyl,
(3) —(CH₂)n-heteroaryl,
(4) —(CH₂)nC₂-₉heterocycloalkyl,
(5) —(CH₂)nC₃-₇cycloalkyl, and
(6) —S(O)₂phenyl,
wherein phenyl, naphthyl, heteroaryl, heterocycloalkyl, cycloalkyl and (CH₂) are unsubstituted or substituted with one to three groups independently selected from R⁹;
each R⁵ is independently selected from the group consisting of
(1) —C₁-₈alkyl,
(2) —(CH₂)ₙphenyl, and
(3) —(CH₂)ₙheteroaryl,
wherein each carbon in —C₁-₈ alkyl is =substituted or substituted with one to three groups independently selected from C₁-₄alkyl;
each R⁶ is independently selected from the group consisting of
(1) hydrogen,
(2) —C₁-₈alkyl,
(3) —C₂-₈alkenyl,
(4) —C₂-₈alkynyl,
(5) (CH₂)ₙphenyl,
(6) —C₂-₈alkenylphenyl, and
(7) —(CH₂)ₙCO₂H
wherein alkyl, alkenyl, alkynyl, and (CH₂)ₙ are unsubstituted or each carbon is substituted with 1 or 2 substituents independently selected from —OC₁-₄alkyl, and —C₁-₄alkyl; and phenyl is unsubstituted or substituted with 1-3 groups selected from —OC₁-₄alkyl, and —C₁-₄alkyl;
each R⁷ is independently selected from the group consisting of:
(1) halogen,
(2) oxo,
(3) ═NH,
(4) —CN,
(5) —CF₃,
(6) —OCF₃,
(7) —C₁-₆, alkyl,
(8) —C₂-₆ alkenyl,
(9) —C₂-₆ alkynyl,
(10) —(CH₂)ₙC₃-₆cycloalkyl,
(11) —(CH₂)ₙC₂-₉heterocycloalkyl,
(12) —(CH₂)ₙOR⁶,
(14) —(CH₂)nCO₂(CH₂)ₙphenyl;
(15) —(CH₂)ₙphenyl;
(16) —(CH₂)ₙ—O-phenyl;
(17) —(CH₂)naphthyl,
(18) —(CH₂)n-heteroaryl
(19) —N(R⁶)₂,
(20) —NR⁶C(O)R⁶,
(21) —NR⁶C(O)₂R⁶,
(22) —C(O)phenyl,
(23) —C(O)heteroaryl.
(24) —SR⁵,
(25) —SO₂C₁-₆alkyl, and
wherein alkyl, alkenyl, alkynyl, phenyl, heteroaryl, heterocycloalkyl, naphthyl, cycloalkyl, and (CH₂)ₙ are unsubstituted or substituted with one to three groups independently selected from oxo, halogen, C₁-₄ alkyl and OR⁵;
each R⁸ is independently selected from the group consisting of:
(1) —C₁-₆alkyl,
(2) C₁-₆alkenyl,
(3) —C₁-₆alkynyl,
(4) —C₁-₆alkoxy,
(5) —C₃-₆cycloalkyl,
(6) —(CH₂)ₙ-phenyl, unsubstituted or substituted with halogen,
(7) —O—(CH₂)ₙ-phenyl,
(8) —CN₅
(9) —OH,
(10) halogen,
(11) —CF₃,
(12) —NH₂,
(13) —N(C₁-₆alkyl)₂,
(14) —NO₂, and
(15) —SC₁-₆alkyl;
each R⁹ is independently selected from the group consisting of:
(1) halogen,
(2) —C₁-₆alkyl,
(3) —C₂-₆alkenyl,
(4) —C₂-₆alkynyl,
(5) -phenyl,
(6) —CH₂phenyl.
(8) —CN,
(9) —OCF₃,
(10) —CF3,
(11) —NO₂,
(12) —NR⁵COR⁵,
(13) —CO₂R⁵, and
(14) —CO₂H;
n is 0, 1, 2, 3, 4, 5, 6, 7 or 8; and
m is 1, 2, 3, 4, 5, 6, 7 or 8;
   compounds having the formula (O):

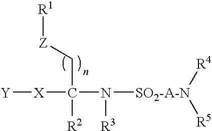

wherein; R1 is aryl, heteroaryl, arylalkyl, heteroarylakyl, cyclyl, cyclylalkyl, heterocyclyl, heterocyclylalkyl, alkyl, alkenyl, alkynyl, each of which is optionally substituted with 1-4 R⁶;
Z is a bond, O, C(O), C(O)O, OC(O), C(O)NR³, NR³C(O), S, SO, SO2, CR²═CR², or C≡C;
n is 1-6, preferably 1-3;
R² is hydrogen, C₁-C₆alkyl, C₂-C₆ alkenyl, or C₂-C₆alkynyl;
R³ is hydrogen, C₁-C₆ alkyl, C₂-C₆alkenyl, or C₂-C₆alkynyl;
A is

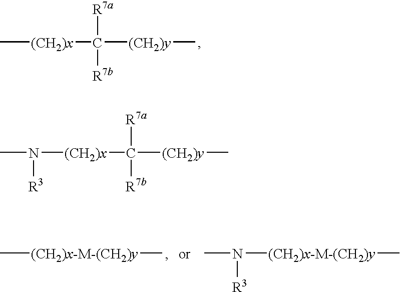

x and y are each independently 0-6;
M is aryl, heteroaryl, cyclyl, or heterocyclyl, each of which is optionally substituted with 1-4 R⁶;
R⁴ and R⁵ are each independently hydrogen, alkyl, alkenyl, haloalkyl, cyclyl, or heterocyclyl, or R⁴ and Wean be taken together to form a heterocyclic ring, or R⁴ and R⁵ can be taken together to form an azido moiety; wherein each R⁴ and R⁵ are optionally substituted with 1-5 halo, 1-3 hydroxy, 1-3 alkyl, 1-3 alkoxy, or 1-3 oxo;
R⁶ is halo, alkyl, cycloalkyl, aryl, heteroaryl, alkoxy, haloalkyl, haloalkyloxy, haloalkylthio, acetyl, cyano, nitro, hydroxy, oxo, C(O)OR², OC(O)R², N(R³)₂, C(O)N(R³)₂, NR³C(O)R², SR²;
R^{7a} and R^{7b} are each independently hydrogen, alkyl, alkenyl, haloalkyl, cycloalkyl, or heterocyclyl, each of which can be optionally substituted with 1-5 halo, 1-3 hydroxy, 1-3 alkyl, or 1-3 alkoxy; or one or both of R^{7a} and R^{7b} can independently be joined to one or both of R⁴ and R⁵ to form one or more bridges between the nitrogen to which the R⁴ and R⁵ are attached and R^{7a} and R^{7b}, wherein each bridge contains 1 to 5 carbons; or one or both of R^{7a} and R^{7b} can independently be joined to one or both of R⁴ and R⁵ to form one or more heterocyclic rings including the nitrogen to which the R⁴ and R⁵ are attached;
X is CH₂CH₂CH₂, wherein one or more CH₂s can be individually replaced with O, C(O), NR³, S, S(O), S(O)₂, or a bond;
Y is

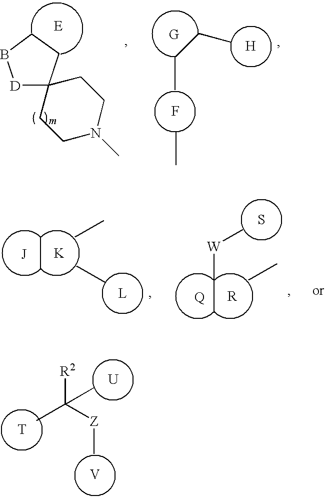

wherein,
B is CHC(O)OR⁸, CHC(O)R⁸, CHC(O)N(R⁸)₂, NSO₂R⁸, CHN(R⁸)₂, C(O), CHN(R⁸)SO₂R⁸, CHCH₂OR⁸, CHR⁸, NR⁸, NC(O)R⁸, NC(O)OR⁸, NC(O)NR³R⁸, or when taken together with D is CR8=CR8;
D is (CH₂)ₚ, CHC₁-C₈ alkyl. O, C(O), or when taken together with B is CR⁸═CR⁸;
wherein p is 1, 2 or 3;
E is independently aryl or heteroaryl, optionally substituted with 1-4 R¹⁰;
m is 0, 1 or 2;
each R⁸ is independently hydrogen, C₁-C₆ alkyl, aryl(C₁-C₆) alkyl, cycloalkyl(C₀-C₆)alkyl, heterocyclyl(C₀-C₆)alkyl, aryl(C₀-C₆)alkyl, or heteroaryl(C₀-C₆)alkyl; each of which can be independently substituted with one or more R⁹;
each R⁹ is independently hydrogen, C₁-C₆ alkyl, aryl(C₁-C₆)alkyl, cycloalkyl(C₀-C₆)alkyl, heterocyclyl(C₀-C₆)alkyl, aryl(C₀-C₆)alkyl, or heteroaryl(C₀-C₆)alkyl, halo, OR⁵, NR⁴SO₂R³, N(R³)₂, CN, C(O)OR², OC(O)R², COR², NO₂, SO₂N(R³)₂, SO₂R³, S(O)R³, SR³, CF³, CH²CF³ or OCF³;
each R¹⁰ is independently halo, C₁-C₆ alkyl, cycloalkyl, aryl, heteroaryl, alkoxy, haloalkyl, haloalkyloxy, haloalkylthio, acetyl, cyano, nitro, hydroxy, C(O)OR², OC(O)R², N(R³)₂. C(O)N(R³)₂, OC(O)N(R³)₂, NR³C(O)OR², NR³C(O)N(R³)₂, NR³C(O)R², or SR²;
each R¹⁰′ is independently halo, C₁-C₆ alkyl, cycloalkyl, aryl, heteroaryl, alkoxy, haloalkyl, haloalkyloxy, haloalkylthio, acetyl, cyano, nitro, hydroxy, oxo, C(O)OR², OC(O)R², N(R³)₂, C(O)N(R³)₂, NR³C(O)R², or SR²;
F and G are each independently aryl or heteroaryl, each of which is optionally substituted with 1-4 R¹⁰, wherein F and H are positioned on adjacent atoms of G;
H is aryl, heteroaryl, heterocyclyl, cyclyl, alkyl, alkenyl, alkynyl, N(R³)₂, OR₂, SR₂, C(O)N(R³)₂, NR³C(O)R₂, CN, N(R³)C(O)OR², R²OC(O)N(R³)alkyl, N(R³)C(O)N(R³)₂, N(R³)₂C(O)N(R³)alkyl, OC(O)N(R³)₂, N(R³)C(O)Oalkyl, or C(O)OR²; each of which is optionally substituted with 1-4 R¹⁰, OR⁵, NR⁴SO₂R³, N(R³)₂, CN, C(O)OR², OC(O)R², COR², NO₂, SO₂N(R³)₂, SO₂R³, S(O)R³, SR³, Cl₃, CH₂CF₃ or OCF₃;
J, K, and L are each independently aryl or heteroaryl, each of which is optionally substituted with 1-4 R¹⁰, wherein X and L are positioned on adjacent atoms of K;
Q, R, and S are each independently aryl, heteroaryl, cyclyl or heterocyclyl, each of which is optionally substituted with R¹⁰, wherein X and S are positioned on non-adjacent atoms of R;
W is CH₂CH₂CH₂, wherein one or more CH₂s can be individually replaced with O, C(O), NR³, S, S(O), S(O)₂, or a bond;
T, U, and V are each independently aryl, heteroaryl, cyclyl or heterocyclyl; each of which is optionally substituted with R¹⁰′; and
Z is CH₂, NR³, O, C(O), S(O), or S(O)₂;
   compounds having the formula (H)

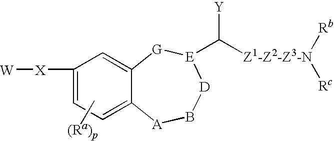

wherein:
A, B, and D are independently selected from the group consisting of a direct bond, —C(R¹XR²)—, —C(R³)═, —C(O)—, —N(R⁴)—, —N═, —O—, and —S(O)ₘ—, wherein in is an integer from 0 to 2, and provided that at least one of A, B, and D is other than a bond; and further provided that when one of A and B is —C(R¹XR²)— and the other is —N(R⁴)—, R⁴ can be optionally combined with R¹, R² or R³ to form a five or six-membered fused ring containing the nitrogen atom to which R⁴ is attached and from 0 to 2 additional heteroatoms selected from the group consisting of N, O and S;
E is N or CH;
R¹, R², R³ and R⁴ are independently selected from the group consisting of hydrogen, halogen, amine, hydroxyl, cyano, (C₁-C₈) allyl, (C₂-C₈) alkenyl, (C₂-C₈) alkynyl, and (C₁-C₈) alkoxy;
G is selected from the group consisting of —C(O) —, —C(S)—, —C(NOR⁵)—, —C(N—NHR⁶)—, and —C(R⁷)(R⁸)—;
Each R¹ is independently selected from the group consisting of halogen, hydroxyl, cyano, (C₁-C₈) alkyl, (C₂-C₈) alkenyl, (C₂-C₈) alkynyl, (C₁-C₈)alkoxy and —NR⁹R¹⁰;
p is an integer from 0 to 3;
X is selected from the group consisting of —C(R¹¹XR¹²)—, —C(O)—, —C(S)—, —O—, —S(O)—, —N(R¹³)—, and —N(OR¹⁴)—, wherein n is an integer from 0 to 2;
R⁵, R⁶, and R¹⁴ are independently selected from the group consisting of hydrogen, (C₁-C₈) alkyl, (C₂-C₈) alkenyl, and (C₂-C₈) alkynyl;
R⁷, R⁸, R⁹, R¹⁰, R¹¹, R¹², and R¹³ are independently selected from the group consisting of hydrogen, (C₁-C₈)alkyl, (C₂-C₈)alkenyl, (C₂-C₈)alkynyl, and (C₁-Cₛ) alkoxy;
W is a ring selected from the group consisting of aryl, heteroaryl, (C₃-C₈) cycloalkyl, (C₅-C₆) heterocycloalkyl, (C₅-C₆) cycloalkenyl, and (C₅-C₆)heterocycloalkenyl;
Y is selected from the group consisting of hydrogen, (C₁-C₈) alkyl, (C₂-C₈) alkenyl, (C₂-C₈) alkynyl, aryl, heteroaryl, (C₃-C₈) cycloalkyl, (C₃-C₈) heterocycloalkyl, (C₅-C₈)cycloalkenyl and (C₅-C₈)heterocycloalkenyl;
Z¹ and Z³ are independently selected from the group consisting of a bond and (C₁-C₈) alkylene;
optionally, Z³ can be combined with R^{b} or R^{c} to form a 3-, 4-, 5-, 6-, 7- or 8-membered ring containing the nitrogen atom to which Z³ is attached and from 0 to 2 additional heteroatoms selected from the group consisting of N, O, and S;
Z² is selected from the group consisting of (C₂-C₈)alkenylene, (C₂-C₈)alkynylene, —C(O)O—, —N(R′)(R″)—, —C(O)N(R′)—, —O—, —S(O)ₖ—, —N(R′)C(O)N(R″)—, N(R′)C(O)O—, —OC(O)O—, arylene, heteroarylene, aryl-(C₁-C₅) alkylene, (C₃-C₈)cycloalkylene, (C₃-C₈) heterocycloalkylene. (C₅-C₈)cycloalkenylene, (C₅-C₈)heterocycloalkenylene, and (C₅-C₈) heterocycloalkylene-C(O)—, wherein k is 0, 1, or 2;
R′ and R″ are independently selected from the group consisting of hydrogen, (C₁-C₈) alkyl, (C₂-C₈) alkenyl, and (C₂-C₈) alkynyl;
R^{b} and R^{c} are independently selected from the group consisting of hydrogen, (C₁-C₈) (C₂-C₈) alkenyl, (C₂-C₈) alkynyl, (C₃-C₈) cycloalkyl, (C₃-C₈) heterocycloalkyl. (C₃-C₈) cycloalkenyl, (C₃-C₈) heterocycloalkenyl, aryl, heteroaryl, halo-(C₁-C₈) alkyl, aryl-(C₁-C₅) alkyl, (C₃-C₈) cycloalkyl-(C₁-C₅)alkyl, (C₃-C₈) heterocycloalkyl-(C₁-C₅) alkyl, (C₃-C₈) heterocycloalkenyl-(C₁-C₅) alkyl, heteroaryl-(C₁-C₅) alkyl, —CR¹⁵CO₂R¹⁶, —CR¹⁵N(R¹⁶)SO₂R¹⁷, —CO₂R¹⁵, —C(O)NR¹⁵R¹⁶, —C(O)NR¹⁵)OR¹⁶, —C(═NOR¹⁵)NR¹⁶R¹⁷, —C(R¹⁵)═NOR¹⁶, —C(O)R¹⁷C(O)NR¹⁵R¹⁶, —NR¹⁵R¹⁶, —NR¹⁵SO₂R¹⁶, —NR¹⁵(OR¹⁶), —NR¹⁷C(O)NR¹⁵C(O)R¹⁶, —NR¹⁵C(O)NR¹⁶R¹⁷, —OR¹⁵, and —SO₂NR¹⁵R¹⁶; optionally, R^{b} and R^{c} may be combined to form a 3-, 4-, 5-, 6-, 7-, or 8-membered ring containing the nitrogen atom to which they are attached from 0 to 3 additional heteroatoms selected from the group consisting of N, O and S; and
R¹⁵, R¹⁶, and R¹⁷ are independently selected from the group consisting of hydrogen, (C₁-C₈) (C₂-C₈) alkenyl, (C₂-C₈) alkynyl, halo-(C₁-C₄)alkyl, hetero(C₁-C₄)alkyl, (C₃-C₈) cycloalkyl, (C₃-C₈) heterocycloalkyl, (C₃-C₈) cycloalkenyl, (C₃-C₈) heterocycloalkenyl, aryl, heteroaryl and aryl-(C₁-C₄)alkyl;
   specific compounds of formula (H) include, but are not limited to:
N-(1-benzyl-piperidin-4-yl)-2-(2,5-dioxo-7-phenoxy-1,2,3,5-tetrahydro-benzo[e][1.4]diazepin-4-yl)-methyl-butyramide,
N-(1-benzyl-piperidin-4-yl)-2-(2,5-dioxo-7-phenoxy-1,2,3,5-tetrahydro-benzo[e][1,4]diazepin-4-yl)-3,3-dimethyl-butyramide,
2-[7-(2,6-dimethyl-phenoxy)-2,5-dioxo-1,2,3,5-tetrahydro-benzo[e][1,4]diazepin-4-yl]-3-methyl-N-[1-(1-phenyl-ethyl)-piperidin-4-yl]-butyramide,
2-[7-(2,6-dimethyl-phenoxy)-2,5-dioxo-1,2,3,5-tetrahydro-benzo[e][1,4]diazepin-4-yl]-N-(1-indan-1-yl-piperidin-4-yl)-3-methyl-butyramide,
4-{1-[2-(1-cyclopropylmethyl-piperidin-4-yl)-ethyl]-2-methyl-propyl}-7-(2,6-difluoro-phenoxy)-3,4-dihydro-1H-benzo[e][1,4]diazepine-2,5-dione,
7-(2-ethyl-phenoxy)-4-{1-[4-(4-fluoro-benzylamino)-piperidine-1-carbonyl]-2-methyl-propyl}-3,4-dihydro-1H-benzo[e][1,4]diazepine-2,5-dione,
4-[1-(4-cyclopropylamino-piperidine-1-carbonyl)-2-methyl-propyl]-7-(2-ethyl-phenoxy)-3,4-dihydro-1H-benzo[e][1,4]diazepine-2,5-dione,
7-(2,4-difluoro-phenoxy)-4-[1-(3-dimethylaminomethyl-phenyl)-2-methyl-propyl]-3,4-dihydro-1H-benzo[e][1,4]diazepine-2,5-dione,
7-(2,4-difluoro-phenoxy)-4-{1-[3-(isopropylamino-methyl)-phenyl]-2-methyl-propyl}dihydro-1H-benzo[e][1,4]diazepine-2,5-dione,
2-(2,5-dioxo-7-phenoxy-1,2,3,5-tetrahydrobenzo[e][1,4]diazepin-4-yl)-3-methyl-butyric acid 1-benzyl-piperidin-4-yl ester,
1-benzyl-piperidine-4-carboxylic acid 2-[7 (4-fluoro-phenoxy)-2,5-dioxo-1,2,3,5-tetrahydro-benzo[e][1,4]diazepin-4-yl]-3-methyl-butyl ester,
(1-benzyl-piperidin-4-yl)-carbamic acid 2-[7-(4-fluoro-phenoxy)-2,5-dioxo-1,2,3,5-tetrahydro-benzo[e][1,4]diazepin-4-yl]-3-methyl-butyl ester,
[1-(4-fluoro-benzyl)-piperidin-4-yl]-carbamic acid 2-[7-(4-fluoro-phenoxy)-2,5-dioxo-1,2,3,5-tetrahydro-benzo[e][1,4]diazepin-4-yl]-3-methyl-butyl ester,
N-(1-benzyl-piperidin-4-yl)-2-(2,5-dioxo-7-phenoxy-1,2,3,5-tetrahydro-benzo[e][1,4]diazepin-4-yl)-3-methyl-butyramide,
7-(2-tert-butyl-phenoxy)-4-[1-(4-cyclopropylamino-piperidine-1-carbonyl)-2-methyl-propyl]-3,4-dihydro-1H-benzo[e][1.4]diazepine-2,5-dione,
N-(1-benzyl-piperidin-4-yl)-2-(2,5-dioxo-7-phenoxy-1,2,3,5-tetrahydro-benzo[e][1,4]diazepin-4-yl)-2-thiophen-2-yl-acetamide,
N-(1-benzyl-piperidin-4-yl)-2-cyclohexyl-2-(2,5-dioxo-7-phenoxy-1,2,3,5-tetrahydro-benzo[e][1,4]diazepin-4-yl)-acetamide,
2-[7-(4-fluoro-phenoxy)-2,5-dioxo-1,2,3,5-tetrahydro-benzo[e][1,4]diazepin-4-yl]-3-methyl-N-(1-phenyl-piperidin-4-yl)-butyramide,
7-(2,6-dimethyl-phenoxy)-4-{1-[4-(4-fluoro-benzylamino)-piperidine-1-carbonyl]-2-methyl-propyl}-3,4-dihydro-1H-benzo[e][1,4]diazepine-2,5-dione.
7-(2,6-dimethyl-phenoxy)-4-(1-(4-[2-(4-fluoro-phenyl)-ethylamino]-piperidine-1-carbonyl-2-methyl-propyl)-3,4-dihydro-1H-benzo[e][1,4]diazepine-2,5-dione,
7-(2-tert-butyl-phenoxy)-4-(1-{4-[2-(4-fluoro-phenyl)-ethylamino]-piperidine-1-carbonyl}-2-methyl-propyl)-3,4-dihydro-1H-benzo[e][1,4]diazepine-2,5-dione,
2-{1-[2-(1-cyclopropylmethyl-piperidine-4-yl)-ethyl]-2-methyl-propyl}-7-(2,4-difluoro-phenoxy)-3,4-dihydro-2H-isoquinolin-1-one
2-[1-(4-cyclopropylamino-piperidine-1-carbonyl)-2-methyl-propyl]-7-(2,4-difluoro-phenoxy)-3,4-dihydro-2H-isoquinolin-1-one,
7-(2,4-difluoro-phenoxy)-2-{1-[4-(4-fluoro-benzylamino)-piperidine-1-carbonyl]-2-methyl-propyl}-3,4-dihydro-2H-isoquinolin-1-one,
7-(2,4-difluoro-phenoxy)-2-(1-{4-[2-(4-fluoro-phenyl)-ethylamino]carbonyl}-2-methyl-propyl)-3,4-dihydro-2H-isoquinolin-1-one,
2-[1-(1-cyclopropylmethyl-piperidin-4-yloxymethyl)-2-methyl-propyl]-7-(2,4-difluoro-phenoxy)-3,4-dihydro-2H-isoquinolin-1-one,
2-(cyclopropyl-{3-[2-(cyclopropylmethyl-amino)-ethyl]-phenyl}-methyl)-7-(2,4-difluoro-phenoxy)-3,4-dihydro-2H-isoquinolin-1-one,
2-{1-[4-(cyclopropylmethyl-amino)-piperidine-1-carbonyl]-2-methyl-propyl}-7-(2,4-difluoro-phenoxy)-3,4-dihydro-2,1-isoquinolin-1-one,
N-(1-benzyl-piperidin-4-yl)-3-methyl-2-(4-oxo-6-o-tolyloxy-4H-quinazolin-3-yl)-butyramide,
N-(1-benzyl-piperidin-4-yl)-2-[8-(4-fluoro-2-methyl-phenoxy)-1-methyl-6-oxo-4H,6H-3,5,10b-triaza-benzo[e]azulen-5-yl]-3-methyl-butyramide,
4-{1-[4-(indan-2-ylamino)-piperidine-1-carbonyl]-2-methyl-propyl}-7-o-tolyloxy-1,2,3,4-tetrahydro-benzo[e][1,4]diazepin-5-one,
8-(2,4-difluoro-phenoxy)-2-{1-[4-(4-fluoro-benzylamino)-piperidine-1-carbonyl]-2-methyl-propyl}2,3-dihydro-benzo[e]azepin-1-one,
2-[1-(4-Cyclopropylamino-cyclohexylmethyl)-2-methyl-propyl]-8-(2,4-difluoro-phenoxy)-2,3,4,5-tetrahydro-benzo[c]azepin-1-one,
2-(1-{4-[(cyclopropylmethyl-amino)-methyl]-thiazol-2-yl}-2-methyl-propyl)-8-(2,4-difluoro-phenoxy)-2,3,4,5-tetrahydro-benzo[e]azepine-1-one,
2-{1-[4-(cyclopropylmethyl-amino)-piperidine-1-carbonyl]-2-methyl-propyl}-8-(2,4-difluoro-phenoxy)-1,2,4,5-tetrahydro-benzo[e]azepin-3-one,
4-(1-{4-[2-(4-fluoro-phenyl)-ethylamino]-piperidine-1-carbonyl}-2-methyl-propyl)-1-methyl-7-o-tolyloxy-1,2,3,4-tetrahydrobenzo[e][1,4]diazepin-5-one,
2-(cyclopropyl(6-((cyclopropylmethylamino)methyl)pyridin-2-yl)methyl)-7-(2,4-difluorophenoxy)-3,4-dihydroisoquinolin-1(2H)-one,
2-(cyclopropyl(6-((cyclopropylamino)methyl)pyridin-2-yl)methyl)-7-(2,4-difluorophenoxy) 3,4-dihydroisoquinolin-1(2H)-one,
2-(cyclopropyl(6-((1-hydroxypropan-2-ylamino)methyl)pyridin-2-yl)methyl)-7-(2,4-difluorophenoxy)-3,4-dihydroisoquinolin-1(2H)-one,
2-(cyclopropyl(6-((1-hydroxy-2-methylpropan-2-ylamino)methyl)pyridin-2-yl)methyl)-7-(2,4-difluorophenoxy)-3,4-dihydroisoquinolin-1(2H)-one,
2-(cyclopropyl(6-((2-fluoroethylamino)methyl)pyridine-2-yl)methyl)-7-(2,4-difluorophenoxy)-3,4-dihydroisoquinolin-1(2H)-one, and
2-(cyclopropyl(6-((2,2-difluoroethylamino) methyl)pyridin-2-yl)methyl)-7-(2 difluorophenoxy)-3,4-dihydroisoquinolin-1(2H)-one;
   or a pharmaceutically acceptable salt or a prodrug of any of the above compounds.

Antibody-based GHS-RAs are also consistent with the claimed method. Anti-GHS-R antibodies may be generated by a variety of well-known methods that include traditional antisera production and monoclonal antibody techniques.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy.

In another embodiment of the invention, an article of manufacture containing materials useful in the presently claimed methods is provided. The article of manufacture comprises a container and a label. Suitable containers include, for example, bottles, vials, syringes, and test tubes. The containers may be formed from a variety of materials such as glass or plastic. The container holds a composition which is effective for specifically inhibiting ghrelin action and may have a sterile access port (for example the container may be an intravenous solution bag or a vial having a stopper pierceable by a hypodermic injection needle).

The active agent in the composition is a GHS-RL, GHS-RA, GHS-RPA and/or GHS-RIA. The label on, or associated with, the container indicates that the composition is used for treating obesity and/or related disorders. The article of manufacture may further comprise a second container comprising a pharmaceutically-acceptable buffer, such as phosphate-buffered saline, Ringer's solution and dextrose solution. It may further include other materials desirable from a commercial end user standpoint, including other buffers, diluents, filters, needles, syringes, and package inserts with instructions for use.

The following examples are offered for illustrative purposes only, and are not intended to limit the scope of the present invention in any way.

### EXAMPLES

The human ghrelin receptor is characterized by a surprisingly high degree of constitutive signalling activity through multiple signalling pathways and that this activity can be inhibited by peptides as well as non-peptide inverse agonists (Hoist et al. Mol. Endocrinology. 2003 17:2201-2210; WO 2004/056869). The high constitutive activity of the ghrelin receptor has opened for novel pharmaco-therapeutic opportunities in developing inverse agonist and partial agonist compounds for the ghrelin receptor.

### Example 1

### Radiolabeled Ligand Competition Binding Assay

Ghrelin binding assays are performed with membrane preparations. CHO-K cells expressing human ghrelin receptor (GHS-R1A) (PerkinElmer) are suspended in sucrose buffer (0.25 M sucrose, 10 mM Hepes pH 7.4, 1 mM PMSF, 5 μg/ml pepstain-A, 3 mM EDTA and 0.025% bacitracin) and disrupted by sonication using e.g. a vibra cell (Sonics and Materials Inc.) on 70% duty cycle in 15-second pulses on ice for 2.5 min. The homogenate is centrifuged at 60,000×g for 60 minutes and pellets are suspended in Tris buffer (20 mM Tris pH 7.4, 5 μg/ml pepstatin-A, 0.1 mM PMSF and 3 mM EDTA).

Binding reactions should contain ˜1 μg membrane as determined by BCA protein assay (Pierce), 0.1 nM [¹²⁵I]-ghrelin (PerkinElmer) with or without compound addition in 100 μl of binding buffer (25 mM Hepes pH 7.4, 1 mM CaCl₂, 5 mM MgSO₄ and 0.5% protease free BSA). Incubations are carried out at room temperature for 2 hr and are terminated by filtration using e.g. a Filtermate Harvester (PerkinElmer) onto GF/C filter plates (Millipore) previously soaked in 0.5% polyethylenimine for 2 hours. Bound [¹²⁵I]-ghrelin is determined by scintillation counting using e.g. a Top Count NXT (PerkinElmer). The effects of compound are expressed as % inhibition of [¹²⁵I]-ghrelin binding. IC50 competitive binding values for the studied compounds are determined by nonlinear regression of the binding curves using e.g. the Prism 3.0 software (GraphPad Software, San Diego).

Known GHS-RA antagonist, e.g. [D-Lys³]-GHRP-6 (H-His-D-Trp-D-Lys-Trp-D-Phe-Lys) which can be purchased from Bachem can be used as a positive control.

### Example 2

### Inositol Phosphate Turnover

The ghrelin receptor signals constitutively through the phospholipase C pathway as determined in spontaneous, ligand-independent stimulation of inositol phosphate turnover. In order to study the ligand independent, spontaneous activity of the ghrelin receptor changes in phospholipase C activity as measured by inositol phosphate turnover is determined in cells transiently transfected with the ghrelin receptor. This method is further used to characterize compounds that can act as ghrelin receptor inverse agonists (GHS-RIA) and ghrelin receptor partial agonists (GHS-RPA).

### Material and Methods

### Compounds

Ghrelin and [D-Arg¹, D-Phe⁵, D-Trp^{7,9}, Leu¹¹]-Substance P can be purchased from Bachem (Bubendorf, Switzerland).

### Molecular Biology

The human ghrelin receptor (GHS-R1A) cDNA (GenBank accession no U60179) can be cloned by PCR from a human brain cDNA library. The cDNA is cloned into a eukaryotic expression vector, e.g. pcDNA3 (Invitrogen, Carlsbad, Calif.).

### Transfections and Tissue Culture

COS-7 cells are grown in Dulbecco's modified Eagle's medium 1885 supplemented with 10% fetal calf serum, 2 mM glutamine and 0.01 mg/ml gentamicin. Cells are transfected using calcium phosphate precipitation method with chloroquine addition.

HEK-293 cells are grown in D-MEM, Dulbecco's modified Eagle's medium 31966 with high glucose supplemented with 10% fetal calf serum, 2 mM glutamine and 0.01 mg/ml gentamicin. Cells are transfected with Lipofectamine 2000 (Life Technologies).

### Phosphatidylinositol Turnover

One day after transfection COS-7 cells are incubated for 24 hours with 5 μCi of [³H]-myo-inositol (Amersham, PT6-271) in 1 ml medium supplemented with 10% fetal calf serum, 2 mM glutamine and 0.01 mg/ml gentamicin per well. Cells are washed twice in buffer, 20 mM HEPES, pH 7.4, supplemented with 140 mM NaCl, 5 mM KCl, 1 mM MgSO₄, 1 mM CaCl₂, 10 mM glucose, 0.05% (w/v) bovine serum; and are incubated in 0.5 ml buffer supplemented with 10 mM LiCl at 37° C. for 30 min. After stimulation with various concentrations of ghrelin receptor ligand for 45 min at 37° C., cells are extracted with 10% ice-cold perchloric acid followed by incubation on ice for 30 min. The resulting supernatants are neutralized with KOH in HEPES buffer, and the generated [³H]-inositol phosphate is purified on e.g. Bio-Rad AG 1-X8 anion-exchange.

Determinations of inositol phosphate accumulation is used as a measure of signalling through the Gq, phospholipase C pathway in COS-7 cells transiently transfected with the human ghrelin receptor. This is used as a measure of the ghrelin receptor activity.

Determination of 1050 for antagonism is made in the presence of 1 μM ghrelin. Determination of 1050 for partial agonism and IC50 for inverse agonism are made in the absence of ghrelin.

### Calculations

IC50 values for antagonism, 1050 values for partial agonism, and IC50 values for inverse agonism are determined by nonlinear regression using e.g. the Prism 3.0 software (GraphPad Software, San Diego).

That the ghrelin receptor signals with an unusually high degree of constitutive activity, can be demonstrated by comparing its activity to that displayed by cells transfected with the empty expression vector.

The constitutive signalling of the ghrelin receptor can be inhibited totally by a potent inverse agonist, e.g. [D-Arg¹, D-Phe⁵, D-Trp^{7,9}, Leu¹¹]-Substance P (Holst et al. supra). This peptide is a low potency antagonist of the ghrelin receptor and a high potency inverse agonist of the ghrelin receptor (GHS-RIA) and thereby serves as an example of compounds having a desired profile of being able to selectively eliminate the ligand-independent signalling of the ghrelin receptor, and thus being an example of compounds which can be used according to the present invention.

The low potency antagonistic effect of [D-Arg¹, D-Phe⁵, D-Trp^{7,9}, Leu¹¹]-Substance P can be confirmed using inositol phosphate accumulation as a measure of the signalling of the ghrelin receptor. The substance P analogue inhibits the ghrelin stimulated inositol phosphate accumulation with an EC50 for antagonism of 630±20 nM (Hoist et al. supra). When [D-Arg¹, D-Phe⁵, D-Trp^{7,9}, Leu¹¹-Substance P is applied to the ghrelin receptor in the absence of ghrelin it can be shown that the peptide also functions as a high efficacy, full inverse agonist as it inhibits the spontaneous, ligand-independent signalling in cells transfected with the ghrelin receptor down to the level observed in cells transfected with the empty expression vector (Holst et al. supra). The potency of [D-Arg¹, D-Phe⁵, D-Trp^{7,9}, Leu¹¹]-Substance P as an inverse agonist can be observed to be 5.2±0.7 nM (Hoist et al. supra), which is approximately 100-fold higher than the potency of the same peptide when studied as an antagonist against ghrelin. Thus [D-Arg¹, D-Phe⁵, D-Trp^{7,9}, Leu¹¹]Substance P is a high potency, high efficacy inverse agonist for the constitutive, ligand-independent signalling of the human ghrelin receptor whereas it functions as a relative low potency antagonist for ghrelin induced signalling.

### Example 3

### CRE and NFAT Reporter Assay

The ghrelin receptor signals constitutively through multiple intracellular pathways as illustrated by the cAMP responsive element (CRE) and the factor of activated T cell (NFAT) gene transcription pathways. The present example can be used to demonstrate that the ghrelin receptor signals constitutively through the downstream cAMP responsive element (CRE) pathway (conceivably activated through some intermediate kinase pathway). In fact the high constitutive signalling activity of the ghrelin receptor can be detected in multiple intracellular signalling pathways. In the present example this is further substantiated by measuring the factor of activated T cell (NFAT) gene transcriptional activity in a reporter assay.

### Material and Methods

(for general molecular pharmacological methods etc. see Example nr. 2)

### CRE and NFAT Reporter Assay.

In both reporter assays HEK293 cells (30 000 cells/well) seeded in 96-well plates are transiently transfected. The indicated amounts of receptor DNA are co-transfected with a mixture of pFA2-CREB and pFR-Luc reporter plasmid (PathDetect CREB trans-Reporting System, Stratagene) in case of the CRE reporter assay and in case of the NFAT reporter assay with pNFAT-luc. One day after transfection, cells are treated with the respective ligands in an assay volume of 100 μl medium for 5 hrs. When treated with the ligands cells are maintained in low serum (2.5%) throughout the experiments. The assay is terminated by washing the cells twice with PBS and addition of 100 μl luciferase assay reagent (LucLite, Packard). Luminescence is measured in e.g. a TopCounter (Top CountNXT, Packard) for 5 sec. Luminescence values are given as relative light units (RLU).

The ghrelin receptor signals constitutively through multiple intracellular signalling pathways. Here, this can be demonstrated by using two reporter assays for respectively cAMP responsive element (CRE) transcriptional activity and for the factor of activated T cell (NFAT) transcriptional activity. The basal, ligand-independent CRE activity can be shown to be increased in transiently transfected cells exposed to increasing amounts of DNA coding for the ghrelin receptor. Thus, the ghrelin receptor in a ligand independent manner stimulates transcriptional activity though the CRE pathway.

### Example 4

### Effects of Central Ghrelin Injection on Alcohol Intake and Alcohol Preference

The following studies sought to determine whether ghrelin increases alcohol intake and the preference for alcohol in mice selected on the basis of their spontaneous level of alcohol intake. We tested the effects of ghrelin injection into the brain ventricles on alcohol consumption and alcohol preference in mice.

### Methods:

All mice used in this study were selected on the basis of their spontaneously medium level of alcohol intake. Initially, the mice were able to choose freely between alcohol solution (10%) and water. When they had established a stable alcohol intake (approximately 20-80% of their total fluid intake was alcohol) they began a training schedule that gave them access to 10% alcohol for 90 min ever day over two weeks. The mice were then implanted with a cannula into the third ventricle of the brain for subsequent injection. During a baseline period, spontaneous alcohol intake was measured during a 90 min period average for 3 measurements taken on 3 days using a two-bottle free choice paradigm (i.e. water or 10% alcohol). The same protocol was used after ghrelin/vehicle injection on the experimental days.

### Results:

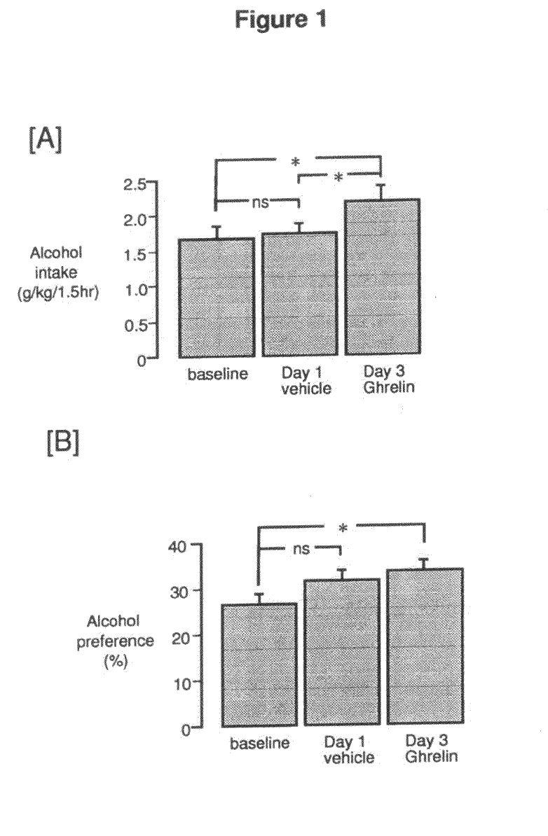

We found that ghrelin injection to the third ventricle (2 μg in 1 μl saline vehicle) significantly increase both alcohol intake and alcohol preference (P<0.05, ANOVA then paired t-test; FIG. 1). Intraventricular administration of ghrelin increased the alcohol intake, compared to the day before the experiments (p<0.05, paired t-test) (FIG. 1A), whereas vehicle treatment did not affect alcohol intake (p>0.05, paired t-test). The average alcohol intake on the day before the experiments (baseline) was 1.65 g/kg/1.5 hr, experiment day one 1.72 g/kg/1.5 hr and day two 2.15 g/kg/1.5 hr. Moreover, a significant increase of alcohol preference was also observed in the ghrelin-treated mice, compared to the preference the day before the experiments (p<0.05, paired t-test) (FIG. 1B). The average preference the day before the experiments was 26.76%, experiment day one 31.72% and day two 33.70%. Vehicle treatment did not significantly affect the preference (p>0.05, paired t-test).

### Example 5

### Effects of Alcohol on Locomotor Activity and Dopamine Release in the Nucleus Accumbens in Ghrelin Receptor (GHS-R1A) Knockout Mice

Stimulation of locomotor activity by alcohol is a well-established method to show activation of the mesolimbic dopamine reward systems. Most drugs of abuse cause increased locomotor activity, an effect mediated, at least in part, by their ability to enhance the extracellular concentration of accumbal dopamine. In this example, we determine whether the effects of alcohol to increase locomotor activity and dopamine release in the nucleus accumbens are altered in ghrelin receptor knockout mice. This will determine whether ghrelin signalling via its receptor (GHS-R1A) is required for alcohol to activate the mesolimbic dopamine system.

### Methods

We tested the effects of alcohol injection (1.0 g/kg, intraperitoneal injection) on locomotor activity in mice lacking the ghrelin receptor (GHS-R1A knockouts) and in wild-type mice. Locomotor activity was registered in eight sound attenuated, ventilated and dimly lit locomotor boxes (420×420×200 mm, Plexiglas®). Five by five rows of photocell beams at the floor level of the box allowed a computer-based system to register the activity of the mice. The mice were allowed to habituate to the environment in the box for one hour before drug challenge and initialization of the experiment. This because naïve animals initially display a high exploratory activity which is followed by a decline in locomotor activity. To reduce the influence of injection-induced hyper-motility, the registration of locomotor activity started 5 minutes after the alcohol administration. Locomotor activity was defined as the accumulated number of new photocell beams interrupted during a 60-minute period.

In vivo microdialysis technique enables measurements of extracellular neurotransmitter levels in the brain in awake, freely moving mice. The method is based on the movement of substances from the outside the probe to the inside. The mice were implanted with a microdialysis probe in the nucleus accumbens for measurements of extracellular dopamine levels. The probe was then connected to a microperfusion pump (II-864 Syringe Pump: AgnThós AB) and perfused with vehicle (Ringer solution) at a rate of 1.5 μl/min. The mice were connected to the microdialysis apparatus via a liquid swivel (CMA/Microdialysis AB, Stockholm, Sweden) and were able to move freely during the experiment. After one hour of habituation to the microdialysis perfusion set up, perfusion samples (30 μl) were collected every 20 minutes. Five samples were collected prior to the first alcohol challenge. The baseline dopamine level is defined as the averaged concentration of the three consecutive samples before the first alcohol challenge. Thereafter vehicle (saline, ip) was administered at time 0 minutes. One hour later, alcohol (1.0 g/kg, ip) was administered and 9 consecutive samples were collected.

### Results

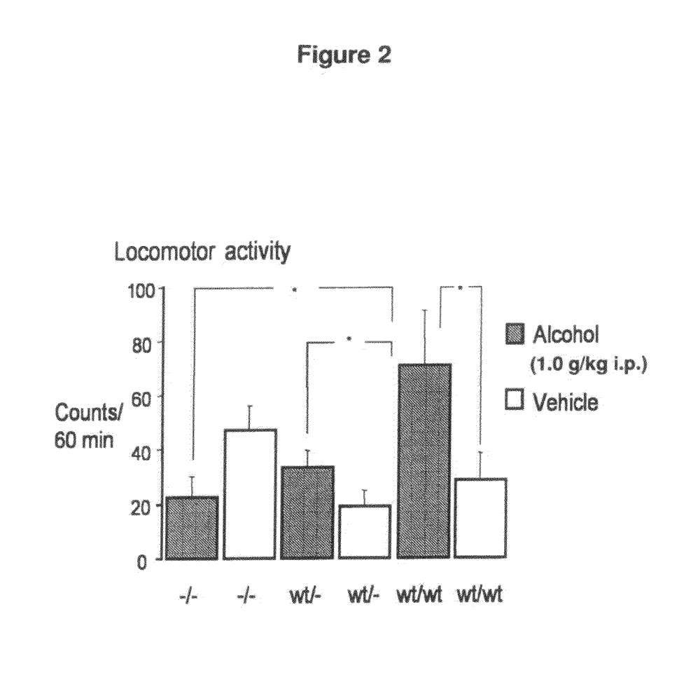

We found that, unlike wildtype mice ghrelin receptor knockout mice do not show an alcohol-induced locomotor activity (P<0.05, Tucky-Kramer following statistically significant ANOVA; FIG. 2).

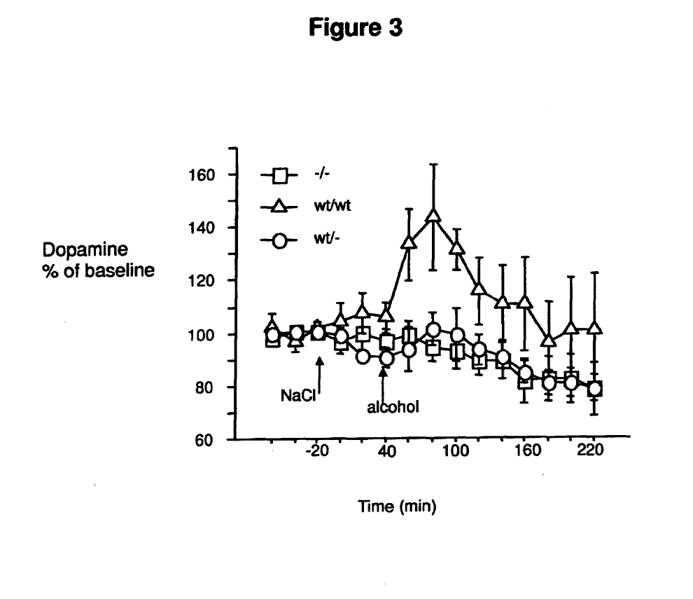

We found that alcohol-induced accumbal dopamine release is blunted in GHSR knockout mice (FIG. 3). There was a significant difference between the wt/wt and wt/− as well as between wt/wt and −/−, data collapsed over the time interval. No difference between −/− and −/wt. Bonferoni followed a statistically significant ANOVA.

### Example 6

### Effects of Central GHS-RL on Alcohol Intake

The following studies can determine whether a GHS-RL is able to suppress alcohol intake and the preference for alcohol in mice selected on the basis of their spontaneous level of alcohol intake. The models used here have been used previously to provide the preclinical basis for the use of drugs currently in use for alcohol-related disorder (e.g. Revia®, Campral®).

The effects of GHS-RL (systemically or locally into the brain) on alcohol consumption and alcohol preference can be tested in mice. All mice used in this study are selected on the basis of their spontaneously level of alcohol intake. Initially, the mice choose freely between alcohol solution (10%) and water. When they establish a stable alcohol intake they are exposed to a training schedule that gives them access to 10% alcohol for 90 min ever day over two weeks, continuous access to water. During a baseline period, spontaneous alcohol intake is measured during a 90 min period average for 3 measurements taken on 3 days using a two-bottle free choice paradigm (i.e. water or 10% alcohol). The same protocol is used after GHS-RL/vehicle injection on the experimental days.

### Example 7

### Effects of Central GHS-RL on Alcohol-Induced Increased Locomotor Activity and Dopamine Release in the Nucleus Accumbens

Stimulation of locomotor activity by alcohol is a well-established method to show activation of the mesolimbic dopamine reward systems. Most drugs of abuse cause increased locomotor activity an effect mediated, at least in part, by their ability to enhance the extracellular concentration of accumbal dopamine. In this example, it is determined whether GHS-RL interfere with alcohol-induced increased locomotor activity and dopamine release in the nucleus accumbens as an indication of suppression of the dopamine reward systems.

The effects of GHS-RL (systemically or locally into the brain) on alcohol (1.0-1.75 g/kg, ip) induced increased locomotor activity in mice is studied. Locomotor activity is registered in eight sound attenuated, ventilated and dimly lit locomotor boxes (420×420×200 mm, Plexiglas®). Five by five rows of photocell beams at the floor level of the box allowed a computer-based system to register the activity of the mice. The mice are allowed to habituate to the environment in the box for one hour before drug challenge and initialization of the experiment. To reduce the influence of injection-induced hyper-motility, the registration of locomotor activity is started 5 minutes after the drug administration. Locomotor activity is defined as the accumulated number of new photocell beams interrupted during a 60-minute period.

In vivo microdialysis technique enables measurements of extracellular neurotransmitter levels in the brain in awake, freely moving mice. The method is based on the movement of substances from the outside the probe to the inside. The mice are implanted with a microdialysis probe in the nucleus accumbens for measurements of extracellular dopamine levels. The probe is then connected to a microperfusion pump (U-864 Syringe Pump: AgnThós AB) and perfused with vehicle (Ringer solution) at a rate of 1.5 μl/min. The mice are connected to the microdialysis apparatus via a liquid swivel (CMA/Microdialysis AB, Stockholm, Sweden) and are able to move freely during the experiment. After one hour of habituation to the microdialysis perfusion set up, perfusion samples (30 μl) is collected every 20 minutes. Five samples are collected prior to the first drug challenge. The baseline DA level is defined as the averaged concentration of the three consecutive samples before the first drug challenge. Thereafter vehicle (saline, ip) is administered at time 0 minutes. One hour later, alcohol (1.0-1.75 g/kg, ip) is administered and 9 consecutive samples are collected.

### Example 8

### Rewarding Effects of Central GHS-RL: Conditioned Place Preference (CPP)

To evaluate if the rewarding effects of alcohol are dependent/influenced by ghrelin signaling, ghrelin-knockout and/or GHS-R-knockout mice are put through a CPP test using alcohol. We expect that ghrelin knockout and GHS-R knockout mice will display less CPP in response to alcohol.

A two-chambered CPP apparatus is used, consisting of two 25×25×25 cm3 compartments with distinct visual and tactile cues. The two compartments are separated by a removable divider. Both compartments are illuminated by dim light with 40-60 lux brightness during the tests. The procedure consists of three different phases: preconditioning (day 1), conditioning (days 2-5), and post-conditioning (day 6). To control possible innate preferences for one of the two conditioning compartments, mice will undergo a single preconditioning session. Immediately after saline injection mice are allowed free access to both conditioning compartments for 20 min. Initial place preference is determined by the side in which a mouse spend more than 600 s out of a 20-min trial. Place preference conditioning is conducted using a biased procedure. The animals are injected with alcohol in the least preferred compartment during the conditioning and saline in the other. Animals are randomly assigned to undergo either drug conditioning in the morning and saline conditioning in the afternoon, or vice versa. Animals receive a total of two injections per day. For drug conditioning, animals are randomly assigned either saline or ethanol (in different doses ranging from 0.5-2.5 g/kg i.p., prepared at 15-20% in saline). Immediately following administration, animals are confined to one of the two conditioning compartments for 20 min. The drug- and saline-paired conditioning compartments and the time of the day of the drug or saline conditioning session (morning or afternoon) are random and counterbalanced across all groups. Conditioning sessions are conducted twice daily for 4 days, with a minimum of 5 h between conditioning sessions.

On the day following the last conditioning session, animals are tested for CPP by placing them between the two compartments (without divider) and are allowed free access to both conditioning compartments for 20 min. CPP is determined by comparing the time spent (in s) in the drug-paired compartment during the preconditioning session and the time spent in the drug-paired compartment during the test (post-conditioning) session.

1. A method for treating chemical substance addiction related disorders in humans comprising administering to a human in need thereof a therapeutically-effective amount of a compound which is a ghrelin receptor ligand (GHS-RL).

2. The method according to claim 1 where the ghrelin receptor ligand (GHS-RL) is a ghrelin receptor inverse agonist (GHS-RIA).

3. The method according to claim 2, wherein the ghrelin receptor inverse agonist (GHS-RIA) is a peptide selected from the group
rPKPfQwFwLL-NH₂
PKPfQwFwLL-NH₂
KPfQwFwLL-NH₂
PfQwFWLL-NH₂
fQwFwLL-NH₂
rPKPAQwFwLL-NH₂
rPKPfAwFwLL-NH₂
rPKPfQwAwLL-NH₂
rPKPfQwFwLA-NH₂
rPKPfQWFwLL-NH₂
rPKPFQwFwLL-NH₂
rPKPyQwFwLL-NH₂
rPKPwQwFwLL-NH₂
rPKP QwFwLL-NH₂.

4. The method according to claim 1 where the ghrelin receptor ligand (GHS-RL) is a ghrelin receptor partial agonist (GHS-RPA).

5. The method according to claim 1 where the ghrelin receptor ligand (GHS-RL) is a ghrelin receptor antagonist (GHS-RA)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. A method for the identification of compound suitable for the treatment of a chemical substance addiction related disorder, said method comprising the steps;
a) providing a test compound;
b) contacting said test compound with a ghrelin receptor;
c) determining the 1050 for inverse agonism, the 1050 for partial agonism and/or the 1050 for antagonism of said test compound for the ghrelin receptor;
d) comparing said 1050 for inverse agonism, the 1050 for partial agonism and/or IC50 for antagonism with the corresponding 1050 values for a known ligand of the ghrelin receptor; and
d) determining that said test compound is suitable for the treatment of a substance addiction related disorder.

20. The method according to claim 19 where the chemical substance addiction related disorder is selected from the group consisting of an alcohol related disorder, cocaine addition, amphetamine addiction, heroin addiction, cannabinoid addiction, and nicotine addiction.

21. The method according to claim 20, wherein the chemical substance addition related disorder is an alcohol related disorder.

22. The method according to claim 5 where the ghrelin receptor antagonist (GHS-RA) is a compound according to formula (D) 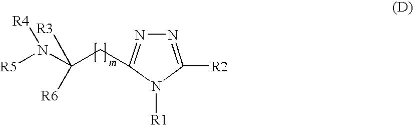
wherein R1 and R2 are independently of one another selected from the group consisting of “hydrogen atom, alkyl, alkenyl, alkynyl, cycloalkyl, cycloalkylalkyl, aryl, heteroaryl, arylalkyl, heteroarylalkyl, heterocyclyl, heterocyclylalkyl, alkylsulfonyl, arylsulfonyl, arylalkylsulfonyl” which are optionally substituted in the alkyl, cycloalkyl, cycloalkylalkyl, aryl, heteroaryl, arylalkyl, heteroarylalkyl, heterocyclyl and/or heterocyclylalkyl group by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”; and preferably are selected from the group consisting of “alkyl, aryl, heteroaryl, arylalkyl, heteroarylalkyl” optionally being substituted by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”;
one of radicals R3 and R4 is a hydrogen atom, whereas the other radical is selected from the group consisting of “hydrogen atom, alkyl, aryl, heteroaryl, arylalkyl, heteroarylalkyl, heterocyclyl, heterocyclylalkyl, -alkyl-O-aryl, -alkyl-O-arylalkyl, -alkyl-O-heteroaryl, -alkyl-O-heteroarylalkyl, -alkyl-O-heterocyclyl, alkyl-O-heterocyclylalkyl, -alkyl-CO-aryl, -alkyl-CO-arylalkyl, -alkyl-CO-heteroaryl, -alkyl-CO-heteroarylalkyl, -alkyl-CO-heterocyclyl, -alkyl-CO-heterocyclylalkyl, -alkyl-C(O)O-aryl, -alkyl-C(O)O-arylalkyl, -alkyl-C(O)O-heteroaryl, -alkyl-C(O)O-heteroarylalkyl, -alkyl-C(O)O-heterocyclyl, -alkyl-C(O)O-heterocyclylalkyl, -alkyl-CO—NH₂, -alkyl-CO—OH, -alkyl-NH₂, -alkyl-NH—C(NH)—NH2, alkylsulfonyl, arylsulfonyl, arylalkylsulfonyl, alkyl-S-alkyl, alkyl-S—H” which are optionally substituted in the aryl, heteroaryl, arylalkyl, heteroarylalkyl, heterocyclyl and/or heterocyclylalkyl group by up to 3 substituents independently selected from the group consisting of “halogen, —F, —Br, —I, —N₃, —CN, —NR7R8, —OH, —NO₂, alkyl, aryl, arylalkyl, —O-alkyl, —O-aryl, —O-arylalkyl”; and preferably are selected from the group consisting of “arylalkyl, heteroarylalkyl, heterocyclylalkyl, -alkyl-O-aryl, -alkyl-O-arylalkyl, -alkyl-O-heteroaryl, -alkyl-O-heteroarylalkyl, -alkyl-O-heterocyclyl, alkyl-O-heterocyclylalkyl, -alkyl-CO-aryl, -alkyl-CO-arylalkyl, -alkyl-CO-heteroaryl, -alkyl-CO-heteroarylalkyl, -alkyl-CO-heterocyclyl, alkyl-CO-heterocyclylalkyl, -alkyl-C(O)O-aryl, -alkyl-C(O)O-arylalkyl, -alkyl-C(O)O-heteroaryl, -alkyl-C(O)O-heteroarylalkyl, -alkyl-C(O)O-heterocyclyl, -alkyl-C(O)O-heterocyclylalkyl, -alkyl-CO—NH₂, -alkyl-CO—OH, -alkyl-NH₂, -alkyl-NH—C(NH)—NH₂,” optionally being substituted in the aryl, heteroaryl, arylalkyl, heteroarylalkyl, heterocyclyl and/or heterocyclylalkyl group by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”;
R5 is selected from the group consisting of “hydrogen atom, alkyl, cycloalkyl, cycloalkylalkyl, aryl, heteroaryl, arylalkyl, heteroarylalkyl, heterocyclyl, heterocyclylalkyl, —CO-alkyl, —CO-cycloalkyl, —CO-cycloalkylalkyl, —CO-aryl, —CO-arylalkyl, —CO-heteroaryl, —CO-heteroarylalkyl, —CO-heterocyclyl, —CO-heterocyclylalkyl, —CO—C*(R9R10)-NH2, —CO—CH2-C*(R9R10)-NH2, —CO—C(R9R10)-CH2-NH2, alkylsulfonyl, arylsulfonyl, arylalkylsulfonyl” which are optionally substituted by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”; and preferably is selected from the group consisting of “hydrogen atom, —CO-alkyl, —CO-cycloalkyl, —CO-aryl, —CO-heteroaryl, —CO-arylalkyl, —CO-heteroarylalkyl, —CO-heterocyclyl, —CO—C*(R9R10)-NH₂, —CO—CH2-C*(R9R10)-NH₂, —CO—C*(R9R10)-CH2-NH₂, optionally being substituted by up to 3 substituents independently selected from the group consisting of “halogen, —F, —Cl, —Br, —I, —N₃, —CN, —NR7R8, —OH, —NO₂, alkyl, aryl, arylalkyl, —O-alkyl, —O-aryl, —O-arylalkyl”;
R6 is selected from the group consisting of “hydrogen atom, alkyl, cycloalkyl, cycloalkylalkyl” and preferably is a hydrogen atom;
R7 and R8 are independently of one another selected from the group consisting of “hydrogen atom, alkyl, cycloalkyl, cycloalkylalkyl” and preferably are a hydrogen atom;
R9 and R10 are independently of one another selected from the group consisting of “hydrogen atom, alkyl, natural alpha-amino acid side chain, unnatural alpha-amino acid side chain” and preferably are selected from the group consisting of “hydrogen atom, alkyl”;
m is 0, 1 or 2 and preferably is 0; and * means a carbon atom of R or S configuration when chiral.

23. The method according to claim 22 where the ghrelin receptor antagonist (GHS-RA) is a compound selected from the group consisting of:
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-phenethyl-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-phenethyl-4H-1,2,4-triazol-3-yl)-2-(1H-indol-3-yl)ethyl)-2-amino-2-methylpropanamide,
(R)—N-(1-(5-benzyl-4-(naphthalen-1-ylmethyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(naphthalen-1-ylmethyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(3-methoxybenzyl)-4H-1,2,4-triazol-3-yl)-2-(1H-indol-3-yl)ethyl)-2-amino-2-methylpropanamide,
(R)—N-(1-(4-(3-methoxybenzyl)-5-benzyl-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-benzyl-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-benzyl-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-(3-methoxybenzyl)-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-(naphthalen-1-ylmethyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(4-methoxybenzyl)-4H-1,2,4-triazol-3-yl)-2-(1H-indol-3-yl)ethyl)-2-amino-2-methylpropanamide,
(R)—N-(1-(4-(4-methoxybenzyl)-5-benzyl-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-(4-bromobenzyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-hexyl-4H-1,2,4-triazol-3-yl)-2-(1H-indol-3-yl)ethyl)-2-amino-2-methylpropanamide,
(R)—N-(1-(5-(3-(1H-indol-3-yl)propyl-4-hexyl-4H-1,2,4-triazol-3-yl)-2-(1H-indol-3-yl)ethyl)-2-amino-2-methylpropanamide,
(R)—N-(1-(4,5-bis(2-(1H-indol-3-yl)ethyl)-4H-1,2,4-triazol-3-yl)-2-(1H-indol-3-yl)ethyl)-2-amino-2-methylpropanamide,
(S)—N-(1-(5-(2-(1H-indol-3-yl)ethyl)-4-(2,4-dimethoxybenzyl)-4H-1,2,4-triazol-3-yl)-2-(1H-indol-3-yl)ethyl)-2-amino-2-methylpropanamide,
(R)—N-(1-(4-(3-methoxybenzyl)-5-phenethyl-4H-1,2,4-triazol-3-yl)-2-(1H-indol-3-yl)ethyl)-2-amino-2-methylpropanamide,
(R)—N-(1-(4-(4-methoxybenzyl)-5-phenethyl-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(3,5-dimethoxybenzyl)-4H-1,2,4-triazol-3-yl)-2-(1H-indol-3-yl)ethyl)-2-amino-2-methylpropanamide,
(R)—N-(1-(4-(4-methoxybenzyl)-5-(3-phenylpropyl)-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-(4-methoxybenzyl)-4H-1,2,4-triazol-3-yl)-2-(1H -indol-3-yl)ethyl)-2-amino-2-methylpropanamide,
(R)—N-(1-(4-(2-(1H-indol-3-yl)ethyl)-5-(3-(1H-indol-3-yl)propyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(2-methoxy)benzyl)-4H-1,2,4-triazol-3-yl)-2-(1H-indol-3-yl)ethyl)-2-amino-2-methylpropanamide,
(R)—N-(1-(4-(2-methoxybenzyl)-5-phenethyl-4H-1,2,4-triazol-3-yl)-2-(1H-indol-3-yl)ethyl)-2-amino-2-methylpropanamide,
(R)—N-(2-(1H-indol-3-yl)-1-(4-(naphthalen-1-ylmethyl)-5-phenethyl-4H-1,2,4-triazol-3-yl)ethyl)-2-amino-2-methylpropanamide,
(R)—N-(1-(5-(2-(1H-indol-3-yl)ethyl)-4-(3,4-dichlorobenzyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(4-fluorobenzyl)-4H-1,2,4triazol-3-yl)-2-(1H-indol-3-yl)ethyl)-2-amino-2-methylpropanamide,
(R)—N-(1-(4-(4-fluorobenzyl)-5-benzyl-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)piperidine-4-carboxamide,
(R)—N-(1-(5-(2-(1H-indol-3-yl)ethyl)-4-(2,4-dimethoxybenzyl)-4H-1,2,4-triazol-3-yl)-2-(1H-indol-3-Yl)ethyl)piperidine-3-carboxamide,
(R)—N-(1-(4-(4-methylbenzyl)-5-(3-phenylpropyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(4-methylbenzyl)-4H-1,2,4-triazol-3-yl)-2-(1H -indol-3-yl)ethyl)-2-amino-2-methylpropanamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)piperidine-2-carboxamide,
(R)—N-(1-(4-(4-methylbenzyl)-5-phenethyl-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)-2-aminobenzamide,
(R)—N-(1-(5-benzyl-4-(pyridin-2-ylmethyl)-4H-1,2,4-triazol-3-yl)-2-(1H-indol-3-yl)ethyl)-2-amino-2-methylpropanamide,
(2S,4R)—N—((R)-1-(4-(4-methoxybenzyl)-5-phenethyl-4H-1,2,4-triazol-3-yl)-2-(1H-indol-3-yl)ethyl)-4-hydroxypyrrolidine-2-carboxamide,
(S)—N—((R)-1-(4-(4-methoxybenzyl)-5-phenethyl-4H-1,2,4-triazol-3-yl)-2-(1H-indol-3-yl)ethyl)piperidine-3-carboxamide,
(R)—N—((R)-1-(4-(4-methoxybenzyl)-5-phenethyl-4H-1,2,4-triazol-3-yl)-2-(1H-indol-3-yl)ethyl)piperidine-3-carboxamide,
(R)—N-(1-(4-(4-ethylbenzyl)-5-phenethyl-4H-1,2,4-triazol-3-yl)-2-(1H-indol-3-yl)ethyl)-2-amino-2-methylpropanamide,
(R)—N-(1-(4-(4-methoxybenzyl)-5-phenethyl-4H-1,2,4-triazol-3-yl)-2-(1H-indol-3-yl)ethyl)piperidine-4-carboxamide,
(R)—N-(1-(5-(2-(1H-indol-3-yl)ethyl)-4-(4-methoxybenzyl)-4H-1,2,4-triazol-3-yl)-2-(1H-indol-3-yl)ethyl)piperidine-4-carboxamide,
(S)—N—((R)-1-(4-(4-methoxybenzyl)-5-phenethyl-4H-1,2,4-triazol-3-yl)-2-(1H-indol-3-yl)ethyl)pyrrolidine-2-carboxamide,
(R)—N—((R)-1-(4-(4-methoxybenzyl)-5-phenethyl-4H-1,2,4-triazol-3-yl)-2-(1H-indol-3-yl)ethyl)pyrrolidine-2-carboxamide,
(S)—N—((R)-1-(4-(4-methoxybenzyl)-5-phenethyl-4H-1,2,4-triazol-3-1)-2-(1H-indol-3-yl)ethyl)piperidine-2-carboxamide,
(R)—N—((R)-1-(4-(4-methoxybenzyl)-5-phenethyl-4H-1,2,4-triazol-3-yl)-2-(1H-indol-3-yl)ethyl)piperidine-2-carboxamide,
(R)—N-(1-(4-(4-methoxybenzyl)-5-phenethyl-4H-1,2,4-triazol-3-yl)-2-(1H-indol-3-yl)ethyl)-2-aminoacetamide,
(R)—N-(1-(4-(4-methoxybenzyl)-5-phenethyl-4H-1,2,4-triazol-3-yl)-2-(1H-indol-3-yl)ethyl)-2-(pyri din-2-yl)acetamide,
(R)—N-(1-(4-(4-methoxybenzyl)-5-phenethyl-4H-1,2,4-triazol-3-yl)-2-(1H-indol-3-yl)ethyl)-2-(pyridin-4-yl)acetamide,
(R)—N-(1-(4-(4-methoxybenzyl)-5-phenethyl-4H-1,2,4-triazol-3-yl)-2-(1H-indol-3-yl)ethyl)cyclohexanecarboxamide,
(R)—N-(1-(5-(2-(1H-indol-3-yl)ethyl)-4-benzyl-4H-1,2,4-triazol-3-yl)-2-(1H-indol-3-yl)ethyl)piperidine-4-carboxamide,
(R)—N-(1-(5-(2-(1H-indol-3-yl)ethyl)-4-benzyl-4H-1,2,4-trioazol-3-yl)-2-(1H-indol-3-yl)ethyl)-3-aminopropanamide,
(R)—N-(1-(4-(4-methoxybenzyl)-5-phenethyl-4H-1,2,4-triazol-3-yl)-2-(1H-indol-3-yl)ethyl)-3-aminopropanamide,
(S)—N—((R)-1-(4-(4-methoxybenzyl)-5-phenethyl-4H-1,2,4-triazol-3-yl)-2-(1H-indol-3-yl)ethyl)-2-aminopropanamide,
(R)—N-(1-(4-(4-methoxybenzyl)-5-phenethyl-4H-1,2,4-triazol-3-yl)-2-(1H-indol-3-yl)ethyl)-2-(pyridin-3-yl)acetamide,
(R)—N-(1-(4-(4-methoxybenzyl)-5-phenethyl-4H-1,2,4-triazol-3-yl)-2-(1H-indol-3-yl)ethyl)-3-(pyridin-3-yl)propanamide,
(R)—N-(1-(5-(2-(1H-indol-3-yl)ethyl)-4-benzyl-4H-1,2,4-triazol-3-yl)-2-(1H-indol-3-yl)ethyl)-2-(pyridin-2-yl)acetamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)-2-(pyridin-2-yl)acetamide,
(R)—N-(1-(4-(2,4-dimethoxybenzyl)-5-phenethyl-4H-1,2,4-triazol-3-yl)-2-(1H-indol-3-yl)ethyl)piperidine-4-carboxamide,
(R)—N-1-(4-(2,4-dimethoxybenzyl)-5-phenethyl-4H-1,2,4-triazol-3-yl)-2-(1H-indol-3-yl)ethyl)piperidine-2-carboxamide,
(R)—N-(1-(4-(2,4-dimethoxybenzyl)-5-phenethyl-4H-1,2,4-triazol-3-yl)-2-(1H-indol-3-yl)ethyl)picolinamide,
(R)—N-(1-(4-(2,4-dimethoxybenzyl)-5-phenethyl-4H-1,2,4-triazol-3-yl)-2-(1H-indol-3-yl)ethyl)isonicotinamide,
(R)—N-(1-(4-(2,4-dimethoxybenzyl)-5-phenethyl-4H-1-yl)-2-(1H-indol-3-yl)ethyl)pyrazine-2-carboxamide,
(R)—N-1-(4-(2,4-dimethoxybenzyl)-5-phenethyl-4H-1,2,4-triazol-3-yl)-2-(1H-indol-3-yl)ethyl)piperazine-2-carboxamide,
(R)—N-1-(4-(2,4-dimethoxybenzyl)-5-phenethyl-4H-1,2,4-triazol-3-yl)-2-(1H-indol-3-yl)ethyl)pyrrolidine-2-carboxamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)-2-aminoacetamide,
(S)—N—((R)-1-(5-(2-(1 W-indol-3-yl)ethyl)-4-(2,4-dimethoxybenzyl)-4H-1,2,4-triazol-3-yl)-2-(1H-indol-3-yl)ethyl)pyrrolidine-2-carboxamide
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)pyrazine-2-carboxamide,
(R)—N-1-(5-(2-(1H-indol-3-yl)ethyl)-4-(2,4-dimethoxybenzyl)-4H-1,2,4-triazol-3-yl)-2-(1H-indol-3-yl)ethyl)piperazine-carboxamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)picolinamide,
(R)-1-(5-(2-(1H-indol-3-yl)ethyl)-4-(2,4-dimethoxybenzyl)-4H-1,2,4-triazol-3-yl)-2-(1H-indol-3-yl)ethanamine,
(R)—N-(1-(4-(2,4-dimethoxybenzyl)-5-phenethyl-4H-1,2,4-triazol-3-yl)-2-(1H-indol-3-yl)ethyl)-2-aminoacetamide,
(R)—N-(1-(4-(4-methoxybenzyl)-5-phenethyl-4H-1,2,4-triazol-3-yl)-2-(1H-indol-3-yl)ethyl)pyrazine-2-carboxamide,
(R)—N-(1-(4-(4-methoxybenzyl)-5-phenethyl-4H-1,2,4-triazol-3-yl)-2-(1H-indol-3-yl)ethyl)isonicotinamide,
(R)—N-1-(4-(4-methoxybenzyl)-5-phenethyl-4H-1,2,4-triazol-3-yl)-2-(1H-indol-3-yl)ethyl)piperazine-2-carboxamide,
(R)—N-(1-(4-(4-methoxybenzyl)-5-phenethyl-4H-1,2,4-triazol-3-yl)-2-(1H-indol-3-yl)ethyl)picolinamide,
(R)—N-(1-(5-(2-(1H-indol-3-yl)ethyl)-4-(4-methoxybenzyl)-4H-1,2,4-triazol-3-yl)-2-(1H-indol-3-yl)ethyl)picolinamide,
(R)—N-(1-(5-(2-(1H-indol-3-yl)ethyl)-4-(4-methoxybenzyl)-4H-1,2,4-triazol-3-yl)-2-(1H-indol-3-yl)ethyl)piperazine-2-carboxamide,
(R)—N-(1-(4-(4-ethylbenzyl)-5-phenethyl-4H-1,2,4-triazol-3-yl)-2-(1H-indol-3-yl)ethyl)-2-(pyridin-2-yl)acetamide,
(R)—N-(1-(4-(4-ethylbenzyl)-5-phenethyl-4H-1,2,4-triazol-3-yl)-2-(1H-indol-3-yl)ethyl)piperidine-4-carboxamide,
(R)—N-1-(4-(4-ethylbenzyl)-5-phenethyl-4H-1,2,4-triazol-3-yl)-2-(1H-indol-3-yl)ethyl)piperazine-2-carboxamide,
(R)—N-(1-(4-(4-ethylbenzyl)-5-phenethyl-4H-1,2,4-triazol-3-yl)-2-(1H-indol-3-yl)ethyl)pyrazine-2-carboxamide,
(R)—N-(1-(5-(2-(1H-indol-3-yl)ethyl)-4-(2,4-dimethoxybenzyl)-4H-1,2,4-triazol-3-yl)-2-(1H-indol-3-yl)ethyl)-2-cis-aminocyclohexanecarboxamide,
(S)—N—((R)-1-(5-(2-(1H-indol-3-yl)ethyl)-4-(4-methoxybenzyl)-4H-1,2,4-triazol-3-yl)-2-(1H-indol-3-yl)ethyl)piperidine-3-carboxamide)
(R)—N—((R)-1-(5-(2-(1H-indol-3-yl)ethyl)-4-(4-methoxybenzyl)-4H-1,2,4-triazol-3-yl)-2-(1H-indol-3-yl)ethyl)piperidine-2-carboxamicle,
(S)—N—((R)-1-(5-(2-(1H-indol-3-yl)ethyl)-4-(4-methoxybenzyl)-4H-1,2,4-triazol-3-yl)-2-(1H-indol-3-yl)ethyl)pyrrolidine-2-carboxamide,
(R)—N—((R)-1-(5-(2-(1H-indol-3-yl)ethyl)-4-(4-methoxybenzyl)-4H-1,2,4-triazol-3-yl)-2-(1H-indol-3-yl)ethyl)pyrrolidine-2-carboxamide,
(R)—N-(1-(5-(2-(1H-indol-3-yl)ethyl)-4-(4-methoxybenzyl)-4H-1,2,4-triazol-3-yl)-2-(1H-indol-3-yl)ethyl)-2-(pyridin-2-yl)acetamide,
(R)—N-(1-(5-(2-(1H-indol-3-yl)ethyl)-4-(4-bromobenzyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(4-methoxybenzyl)-4H-1,2,4-triazol-3-yl)-2-phenylethyl)-2-amino-2-methylpropanamide,
(R)—N-(2-(1H-indol-3-yl)-1-(5-phenethyl-4-(thiophen-2-ylmethyl)-4H-1,2,4-triazol-3-yl)ethyl)piperidine-4-carboxamide,
(R)—N-(1-(4-(2-(1H-indol-3-yl)ethyl)-4H-1,2,4-triazol-3-yl)-2-(1H-indol-3-yl)ethyl)-2-amino-2-methylpropanamide,
(R)—N-(1-(5-((1H-indol-3-yl)methyl)-4-methyl-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-methyl-4H-1,2,4-triazol-3-yl)-2-(1H-indol-3-yl)ethyl)-2-amino-2-methylpropanamide,
(R)—N-(1-(5-((1H-indol-3-yl)methyl)-4H-1,2,4-triazol-3-yl)-2-(1H-indol-3-yl)ethyl)-2-amino-2-methylpropanamide,
(R)—N-(1-(5-((1AV-indol-3-yl)methyl)-4-(2,4-dimethoxybenzyl)-4H-1,2,4-triazol-3-yl)-2-(1H-indol-3-yl)ethyl)-2-amino-2-methylpropanamide,
(R)—N-(1-(4-(2,4-dimethoxybenzyl)-5-methyl-4H-1,2,4-triazol-3-yl)-2-(1H-indol-3-yl)ethyl)-2-amino-2-methylpropanamide,
(R)—N-(1-(5-((1H-indol-3-yl)methyl)-4-(4-methoxybenzyl)-4H-1,2,4-triazol-3-yl)-2-(1H-indol-3-yl)ethyl)-2-amino-2-methylpropanamide,
(R)—N-(1-(4-(2,4-dimethoxybenzyl)-5-benzyl-4H-1,2,4-triazol-3-yl)-2-(1H-indol-3-yl)ethyl)-2-amino-2-methylpropanamide,
(R)—N-(1-(5-(3-(1H-indol-3-yl)propyl)-4-(2,4-dimethoxybenzyl)-4H-1,2,4-triazol-3-yl)-2-(1H-indol-3-yl)ethyl)-2-amino-2-methylpropanamide,
(R)—N-(1-(5-((1H-indol-3-yl)methyl)-4-phenethyl-4H-1,2,4-triazol-3-yl)-2-(1H-indol-3-yl)ethyl)-2-amino-2-methylpropanamide,
(R)—N-(1-(5-benzyl-4-phenethyl-4H-1,2,4-triazol-3-yl)-2-(1H-indol-3-yl)ethyl)-2-amino-2-methylpropanamide,
(R)—N-(1-(5-benzyl-4-(2,2-diphenylethyl)-4H-1,2,4-triazol-3-yl)-2-(1H-indol-3-yl)ethyl)-2-amino-2-methylpropanamide,
(R)—N-(1-(5-(2-(1H-indol-3-yl)ethyl)-4-(2,2-diphenylethyl)-4H-1,2,4-triazol-3-yl)-2-(1H-indol-3-yl)ethyl)-2-amino-2-methylpropanamide,
(R)—N-(1-(4-(3,5-dimethoxybenzyl)-5-benzyl-4H-1,2,4-triazol-3-yl)-2-(1H-indol-3-yl)ethyl)-2-amino-2-methylpropanamide,
(R)—N-(1-(4,5-dibenzyl-4H-1,2,4-triazol-3-yl)-2-(1H-indol-3-yl)ethyl)-2-amino-2-methylpropanamide,
(R)—N-(1-(5-benzyl-4-hexyl-4H-1,2,4-triazol-3-yl)-2-(1H-indol-3-yl)ethyl)-2-amino-2-methylpropanamide,
(R)—N-(1-(4-(2-(1H-indol-3-yl)ethyl)-5-benzyl-4H-1,2,4-triazol-3-yl)-2-(1H-indol-3-yl)ethyl)-2-amino-2-methylpropanamide,
(S)—N-(1-(4-(2,4-dimethoxybenzyl)-5-benzyl-4H-1,2,4-triazol-3-yl)-2-(1H-indol-3-yl)ethyl)-2-amino-2-methylpropanamide,
(R)—N-(1-(4-(3,5-dimethoxybenzyl)-5-phenethyl-4H-1,2,4-triazol-3-yl)-2-(1H-indol-3-yl)ethyl)-2-amino-2-methylpropanamide,
(R)—N-(1-(4-(4-bromobenzyl)-5-benzyl-4H-1,2,4-triazol-3-yl)-2-(1H-indol-3-yl)ethyl)-2-amino-2-methylpropanamide,
(R)—N-(1-(4-(2-methoxybenzyl)-5-benzyl-4H-1,2,4-triazol-3-yl)-2-(1H-indol-3-yl)ethyl)-2-amino-2-methylpropanamide,
(S)—N-(1-(4-(2,4-dimethoxybenzyl)-5-phenethyl-4H-1,2,4-triazol-3-yl)-2-(1H-indol-3-yl)ethyl)-2-amino-2-methylpropanamide,
(R)—N-(1-(4,5-diphenethyl-4H-1,2,4-triazol-3-yl)-2-(1H-indol-3-yl)ethyl)-2-amino-2
(R)—N-(1-(4-(3,4-dichlorobenzyl)-5-benzyl-4H-1,2,4-triazol-3-yl)-2-(1H-indol-3-yl)ethyl)-2-amino-2-methylpropanamide,
(R)-1-(5-(2-(1H-indol-3-yl)ethyl)-4-(2,4-dimethoxybenzyl)-4H-1,2,4-triazol-3-yl)-2-(1H-indol-3-yl)ethanamine,
(R)—N-(1-(4-(4-methoxybenzyl)-5-benzyl-4H-1,2,4-triazol-3-yl)-2-phenylethyl)-2-amino-2-methylpropanamide,
(R)—N-(1-(4-(4-fluorobenzyl)-5-phenethyl-4H-1,2,4-triazol-3-yl)-2-(1H-indol-3-yl)ethyl)-2-amino-2-methylpropanamide,
(R)—N-(1-(4-(3,4-dichlorobenzyl)-5-phenethyl-4H-1,2,4-triazol-3-yl)-2-(1H-indol-3-yl)ethyl)-2-amino-2-methylpropanamide,
(R)—N-(1-(4-(4-methylbenzyl)-5-benzyl-4H-1,2,4-triazol-3-yl)-2-(1H-indol-3-yl)ethyl)-2-amino-2-methylpropanamide,
(S)—N-(1-(4-(4-methoxybenzyl)-5-(3-phenylpropyl)-4H-1,2,4-triazol-3-yl)-2-(1H-indol-3-yl)ethyl)-2-amino-2-methylpropanamide,
(S)—N-(1-(4-(4-methoxybenzyl)-5-benzyl-4H-1,2,4-triazol-3-yl)-2-(1H-indol-3-yl)ethyl)-2-amino-2-methylpropanamide,
N—((R)-1-(4-(4-nitrobenzyl)-5-phenethyl-4H-1,2,4-triazol-3-yl)-2-(1H-indol-3-yl)ethyl)-2-amino-2-methylpropanamide,
(S)—N-(1-(4-(4-methoxybenzyl)-5-phenethyl-4H-1,2,4-triazol-3-yl)-2-(1H-indol-3-yl)ethyl)-2-amino-2-methylpropanamide,
(R)—N-(1-(4-(4-methoxyphenethyl)-5-phenethyl-4H-1,2,4-triazol-3-yl)-2-(1H-indol-3-yl)ethyl)-2-amino-2-methylpropanamide,
(R)—N-(2-(1H-indol-3-yl)-1-(5-phenethyl-4-(thiophen-2-ylmethyl)-4H-1,2,4-triazol-3-yl)ethyl)-2-amino-2-methylpropanamide,
(R)—N-(2-(1H-indol-3-yl)-1-(5-phenethyl-4-(pyridin-2-ylmethyl)-4H-1,2,4-triazol-3-yl)ethyl)-2-amino-2-methylpropanamide,
(R)—N-(2-(1H-indol-3-yl)-1-(5-phenethyl-4-(pyridin-2-ylmethyl)-4H-1,2,4-triazol-3-yl)ethyl)piperidine-3-carboxamide,
(S)—N-1-(4-(4-ethylbenzyl)-5-phenethyl-4H-1,2,4-triazol-3-yl)-2-(1H-indol-3-yl)ethyl)pyrrolidine-2-carboxamide,
N—((R)-1-(4-(4-ethylbenzyl)-5-phenethyl-4H-1,2,4-triazol-3-yl)-2-(1H-indol-3-yl)ethyl)-2-aminoacetamide,
N—((R)-1-(5-(2-(1H-indol-3-yl)ethyl)-4-(4-methoxybenzyl)-4H-1,2,4-triazol-3-yl)-2-(1H-indol-3-yl)ethyl)-2-(pyridin-4-yl)acetamide,
(2R)—N—((R)-1-(4-(4-ethylbenzyl)-5-phenethyl-4H-1,2,4-triazol-3-yl)-2-(1H-indol-3
N—((R)-1-(4-(4-ethylbenzyl)-5-phenethyl-4H-1,2,4-triazol-3-yl)-2-(1H-indol-3-yl)ethyl)picolinamide,
N—((R)-1-(4-(4-ethylbenzyl)-5-phenethyl-4H-1,2,4-triazol-3-yl)-2-(1H-indol-3-yl)ethyl)-2-aminopyridine-3-carboxamide,
(2S)—N—((R)-1-(4-(4-ethylbenzyl)-5-phenethyl-4H-1,2,4-triazol-3-yl)-2-(1H-indol-3-yl)ethyl)-2-aminopropanamide,
N—((R)-1-(4-(4-ethylbenzyl)-5-phenethyl-4H-1,2,4-triazol-3-yl)-2-(1H-indol-3-yl)ethyl)isonicotinamide,
N—((R)-2-(1H-indol-3-yl)-1-(5-phenethyl-4-phenyl-4H-1,2,4-triazol-3-yl)ethyl)piperidine-4-carboxamide,
(2S)—N—((R)-2-(1H-indol-3-yl)-1-(5-phenethyl-4-phenyl-4H-1,2,4-triazol-3-yl)ethyl)pyrrolidine-2-carboxamide,
N—((R)-2-(1H-indol-3-yl)-1-(5-phenethyl-4-phenyl-4H-1,2,4-triazol-3-yl)ethyl)-2-aminoacetamide,
N—((R)-2-(1H-indol-3-yl)-1-(5-phenethyl-4-phenyl-4H-1,2,4-triazol-3-yl)ethyl)-2-(pyridin-2-yl)acetamide,
N—((R)-2-(1H-indol-3-yl)-1-(5-phenethyl-4-phenyl-4H-1,2,4-triazol-3-yl)ethyl)picolinamide,
N—((R)-1-(5-(2-(1H-indol-3-yl)ethyl)-4-(4-ethylphenyl)-4H-1,2,4-triazol-3-yl)-2-(1H-indol-3-yl)ethyl)picolinamide,
N—((R)-1-(5-(2-(1H-indol-3-yl)ethyl)-4-(4-ethylphenyl)-4H-1,2,4-triazol-3-yl)-2-(1H-indol-3-yl)ethyl)-2-(pyridin-2-yl)acetamide,
N—((R)-1-(5-(2-(1H-indol-3-yl)ethyl)-4-(4-ethylphenyl)-4H-1,2,4-triazol-3-yl)-2-(1H-indol-3-yl)ethyl)-2-aminoacetamide,
(2S)—N—((R)-1-(5-(2-(1H-indol-3-yl)ethyl)-4-(4-ethylphenyl)-4H-1,2,4-triazol-3-yl)-2-(1H-indol-3-yl)ethyl)pyrrolidine-2-carboxamide,
N—((R)-1-(5-(2-(1H-indol-3-yl)ethyl)-4-(4-methoxybenzyl)-4H-1,2,4-triazol-3-yl)-2-(1H-indol-3-yl)ethyl)-2-aminoacetamide,
N—((R)-1-(4-(4-methoxybenzyl)-5-phenethyl-4H-1,2,4-triazol-3-yl)-2-(1H-indol-3-yl)ethyl)-2-trans-aminocyclohexanecarboxamide,
N—((R)-1-(4-(4-ethylbenzyl)-5-phenethyl-4H-1,2,4-triazol-3-yl)-2-(1H-indol-3-yl)ethyl)-2-(pyridin-3-yl)acetamide,
(3S)—N—((R)-1-(4-(4-ethylbenzyl)-5-phenethyl-4H-1,2,4-triazol-3-yl)-2-(1H-indol-3-yl)ethyl)piperidine-3-carboxamide,
N—((R)-1-(4-(4-ethylbenzyl)-5-phenethyl-4H-1,2,4-triazol-3-yl)-2-(1H-indol-3-yl)ethyl)-2-aminobenzamide,
N—((R)-1-(5-(2-(1H-indol-3-yl)ethyl)-4-phenyl-4H-1,2,4-triazol-3-yl)-2-(1H-indol-3-yl)ethyl)picolinamine,
N—((R)-1-(5-(2-(1H-indol-3-yl)ethyl)-4-phenyl-4H-1,2,4-triazol-3-yl)-2-(1H-indol-3-yl)ethyl)piperidine-4-carboxamide,
N—((R)-2-(1H-indol-3-yl)-1-(4-(2,4-dimethoxyphenyl)-5-phenethyl-4H-1,2,4-triazol-3-yl)ethyl)picolinamide,
N—((R)-2-(1H-indol-3-yl)-1-(4-(2,4-dimethoxyphenyl)-5-phenethyl-4H-1,2,4-triazol-3-yl)ethyl)-2-(pyridin-2-yl)acetamide,
N—((R)-2-(1H-indol-3-yl)-1-(4-(2,4-dimethoxyphenyl)-5-phenethyl-H-1,2,4-triazol-3-yl)ethyl)pyrazine-2-carboxamide,
N—((R)-2-(1H-indol-3-yl)-1-(4-(2,4-dimethoxyphenyl)-5-phenethyl-4H-1,2,4-triazol-3-yl)ethyl)-2-aminoacetamide,
N—((R)-2-(1H-indol-3-yl)-1-(4-(2,4-dimethoxyphenyl)-5-phenethyl-4,1-,2,4-triazol-3-yl)ethyl)piperidine-4-carboxamide,
N—((R)-1-(5-benzyl-4-((pyri din-2-yl)methyl)-4H-1,2,4-triazol-3-yl)-2-(1H-indol-3-yl)ethyl)picolinamide,
N—((R)-1-(5-benzyl-4-((pyridin-2-yl)methyl)-4H-1,2,4-triazol-3-yl)-2-1H-indol-3-yl)ethyl)-2-amino-acetamide,
N—((R)-1-(5-benzyl-4-((pyridin-2-yl)methyl)-4H-1,2,4-triazol-3-yl)-2-(1H-indol-3-yl)ethyl)piperidine-4-carboxamide,
N—((R)-1-(5-benzyl-4-((pyridin-4-yl)methyl)-4H-1,2,4-triazol-3-yl)-2-(1H-indol-3-yl)ethyl)-2-amino-2-methylpropanamide,
N—((R)-1-(5-(4-methoxybenzyl)-4-phenethyl-4H-1,2,4-triazol-3-yl)-2-(1H-indol-3-yl)ethyl)-2-amino-2-methylpropanamide,
N—((R)-1-(5-benzyl-4-((pyridin-4-yl)methyl)-4H-1,2,4-triazol-3-yl)-2-(1H-indol-3-yl)ethyl)picolinamide,
N—((R)-1-(5-benzyl-4-((pyridin-4-yl)methyl)-4H-1,2,4-triazol-3-yl)-2-(1H-indol-3-yl)ethyl)-2-amino-acetamide,
(R)-benzyl-3-(2-aminoisobutyramido)-3-(2-(1H-indol-3-yl)ethyl)-4-(4-methoxybenzyl)-4H-1,2,4-triazol-3-yl)-propanoate,
N—((R)-1-(5-benzyl-4-((pyri din-3-yl)methyl)-4H-1,2,4-triazol-3-yl)-2-(1H-indol-3-yl)ethyl)-2-amino-2-methylpropanamide,
N—((R)-1-(4-benzyl-5-phenethyl-4H-1,2,4-triazol-3-yl)-2-(1H-indol-3-yl)ethyl)-2-amino-2-methylpropanamide,
N—((R)-2-(1H-indol-3-yl)-1-(4-methyl-5-phenethyl-4H-1,2,4-triazol-3-yl)ethyl)picolinamide,
N—((R)-1-(5-(2-(1H-indol-3-yl)ethyl)-4-phenyl-4H-1,2,4-triazol-3-yl)-2-(1H-indol-3-yl)ethyl)-2-amino-2-methylpropanamide,
N—((R)-1-(4-(4-methoxybenzyl)-5-phenethyl-4H-1,2,4-triazol-3-yl)-2-(1H-indol-3-yl)ethyl)benzamide,
(R)-1-(4-(2,4-dimethoxybenzyl)-5-phenethyl-4H-1,2,4-triazol-3-yl)-2-(1H-indol-3-yl)-N-phenylmethanesulfonylamine,
(R)-1-(4-(2,4-dimethoxyben2yl)-5-phenethyl-4H-1,2,4-triazol-3-yl)-2-(1H-indol-3-yl)-N-tosylethanamine,
N—((R)-1-(4-(2,4-dimethoxybenzyl)-5-phenethyl-4H-1,2,4-triazol-3-yl)-2-(1H-indol-3-yl)ethyl)-2-amino-2-methylpropanamide,
N-1-((R)-1-(4-(2,4-dimethoxybenzyl)-5-phenethyl-4H-1,2,4-triazol-3-yl)-2-(1H-indol-3-yl)ethyl)ethane-1,2-diamine,
N—((R)-1-(4-((furan-2-yl)methyl)-5-phenethyl-4H-1,2,4-triazol-3-yl)-2-(1H-indol-3-yl)ethyl)-2-amino-2-methylpropanamide,
N—((R)-1-(4-((furan-2-yl)methyl)-5-phenethyl-4H-1,2,4-triazol-3-yl)-2-(1H-indol-3-yl)ethyl)picolinamide,
N—((R)-1-(4-((furan-2-yl)methyl)-5-phenethyl-4H-1,2,4-triazol-3-yl)-2-(1H-indol-3-yl)ethyl)piperidine-4-carboxamide,
N—((R)-1-(4-(4-methoxybenzyl)-5-phenethyl-4H-1,2,4-triazol-3-yl)-2-(1H-indol-3-yl)ethyl)-tetrahydro-2W-pyran-4-carboxamide
N—((R)-1-(5-((1H-indol-3-yl)methyl)-4-(3-methoxybenzyl)-4H-1,2,4-triazol-3-yl)-2-(1H-indol-3-yl)ethyl)-2-amino-2-methylpropanamide,
(2S)—N—((R)-1-(4-(4-methoxybenzyl)-5-phenethyl-4H-1,2,4-triazol-3-yl)-2-(1H-indol-3-yl)ethyl)-2-amino-3-phenylpropanamide,
(R)-1-(5-(2-(1H-indol-3-yl)ethyl)-4-(2,4-dimethoxybenzyl)-4H-1,2,4-triazol-3-yl)-2-(1H-indol-3-yl)-N-tosylethanamine,
N—((R)-1-(4-(2,4-dimethoxybenzyl)-5-phenethyl-4H-1,2,4-triazol-3-yl)-2-(1H-indol-3-yl)ethyl)-4-azidobenzamide,
N-benzyl-(R)-1-(4-(2,4-dimethoxybenzyl)-5-phenethyl-4H-1,2,4-triazol-3-yl)-2-(1H-indol-3-yl)ethanamine,
(2S)—N—OR)-1-(5-(2-(1H-indol-3-yl)ethyl)-4-(4-methoxybenzyl)-4H-1,2,4-triazol-3-yl)-2-(1H-indol-3-yl)ethyl)-2,5-dihydro-1H-pyrrole-2-carboxamide.

24. The method according to claim 23 wherein the ghrelin receptor antagonist (GHS-RA) is the compound
(R)—N-(1-(4-(4-methoxybenzyl)-5-phenethyl-4H-1,2,4-triazol-3-yl)-2-(1H-indol-3-yl)ethyl)-2-aminoacetamide.

25. The method according to claim 1, wherein the chemical substance addiction related disorder is selected from the group consisting of an alcohol related disorder, cocaine addiction, amphetamine addiction, heroin addiction, and nicotine addiction.

26. The method according to claim 25, wherein the chemical substance addiction related disorder is an alcohol related disorder.
